(19) United States
(12) Patent Application Publication
(10) Pub. No.: US 2021/0209959 A1
(43) Pub. Date: Jul. 8, 2021

(54) Method and Apparatus for Personal Awareness and Growth

(71) Applicant: Geoffrey S. Lycas, Castle Rock, CO (US)

(72) Inventor: Geoffrey S. Lycas, Castle Rock, CO (US)

(21) Appl. No.: 17/025,662

(22) Filed: Sep. 18, 2020

Related U.S. Application Data
(63) Continuation-in-part of application No. 16/056,649, filed on Aug. 7, 2018, now Pat. No. 10,963,977.
Application No. 16/056,649 is a continuation-in-part of application No. 15/429,606, filed on Feb. 10, 2017, now Pat. No. 10,055,802.
Application No. 15/429,606 is a continuation of application No. 13/896,553, filed on May 17, 2013, now Pat. No. 9,589,475.
Application No. 13/896,553 is a continuation of application No. 13/538,431, filed on Jun. 29, 2012, now Pat. No. 8,449,300.
Application No. 13/538,431 is a continuation of application No. 12/987,555, filed on Jan. 10, 2011, now Pat. No. 8,226,418.
Application No. 12/987,555 is a continuation of application No. 10/896,438, filed on Jul. 21, 2004, now Pat. No. 7,874,841.
Application No. 10/896,438 is a continuation-in-part of application No. 10/367,074, filed on Feb. 13, 2003, now abandoned.
Application No. 10/367,074 is a continuation-in-part of application No. 10/215,954, filed on Aug. 8, 2002, now abandoned.
(60) Provisional application No. 60/310,886, filed on Aug. 8, 2001.

(51) Int. Cl. G09B 7/00; G09B 5/06; G09B 7/02; G09B 23/28; G09B 7/04; G06Q 30/02; G09B 19/00; G16H 20/70; G09B 5/00

(52) U.S. Cl. CPC G09B 7/00; G09B 5/065; G09B 5/06; G09B 7/02; G09B 23/28; G09B 5/00; G06Q 30/0269; G06Q 30/0245; G09B 19/00; G16H 20/70; G09B 7/04

(57) ABSTRACT

An presentation generator is disclosed for generating presentations for interacting with a user on a personal topic of, e.g., the user's selection, wherein the presentations assist the user in obtaining a greater awareness of his/her motivations and/or behaviors relating to the topic. In one embodiment, the presentation generator generates and presents to the user textual observations, questions, and/or statements for the user's consideration. Such presentations use and/or are consistent with textual descriptions obtained from: (a) the results of one or more personality/motivation test results, and (b) user inputs, e.g., regarding the selected topic together with his/her confidence in the validity of such inputs. The invention organizes the textual descriptions so that outputs to the user can be generated from various personality/motivational perspectives thereby assisting the user in viewing the topic of discussion from different perspectives and thereby becoming more aware of his/her biases, motivations, and/or concerns relating to the topic.

### RELATED APPLICATIONS

The present patent application is a continuation-in-part of application Ser. No. 16/056,649, filed Aug. 7, 2018, which is a continuation-in-part of application Ser. No. 15/429,606, filed Feb. 10, 2017 (now U.S. Pat. No. 10,055,802, issuing Aug. 21, 2018), which is a continuation of U.S. patent application Ser. No. 13/896,553, filed on May 17, 2013 (now U.S. Pat. No. 9,589,475, issued Mar. 7, 2017), which is a continuation of U.S. patent application Ser. No. 13/538,431, filed on Jun. 29, 2012 (now U.S. Pat. No. 8,449,300, issued May 28, 2013), which is a continuation of U.S. patent application Ser. No. 12/987,555, filed Jan. 10, 2011 (now U.S. Pat. No. 8,226,418, issued Jul. 24, 2012), which in turn is a continuation of U.S. patent application Ser. No. 10/896,438, filed Jul. 21, 2004 (now U.S. Pat. No. 7,874,841, issued Jan. 25, 2011), which in turn is a continuation-in-part of U.S. patent application Ser. No. 10/367,074 filed Feb. 13, 2003, which in turn is a continuation-in-part of U.S. patent application Ser. No. 10/215,954 filed Aug. 8, 2002, which claims the benefit of U.S. Provisional Patent Application Ser. No. 60/310,886 filed Aug. 8, 2001. The entire disclosure of the prior applications are incorporated by reference herein and is considered to be part of the disclosure of the present application.

### FIELD OF THE INVENTION

The present application relates generally to a computational system and method for facilitating personal growth and/or insights. More particularly, the present invention provides a method and system for interacting with a user using presentations that are customized to both a personal profile of the user as well as the user's understanding of particular words and/or phrases for gaining such personal growth and/or insights. In at least one embodiment, the invention computationally combines: (a) semantic linkages between words and/or phrases with (b) a novel syntactic analysis to produce textual output for a designated discussion topic, wherein the textual output is generated using a user requested view point on the topic.

### BACKGROUND OF THE INVENTION

Every living thing that exists has energy that integrally supports the life process in all its aspects—the material operations of the physical body, the functions of the emotions and mind, and even the spiritual life. Every individual has a different energy pattern and growth potential that fluctuates through each day and throughout the individual's lifetime. To be able to take advantage of or to recognize the fluctuations in one's energy levels could greatly increase the individual's productivity and well-being, both at work and at home, and would enhance the individual's growth. The prior art discusses different ways of assessing an individual's energy and potential for growth; however, such assessments are not necessarily an exact science. Indeed, the intension of the prior art is to identify a method that quantifies the energy pattern and/or growth potential of an individual. The prior art recommends that an “energy healer” (denoted as “facilitator” herein) or one skilled in the art of addressing an individual's energy to diagnose or address an individual's energy functions and potential dysfunctions and to use the combination of the healer's and individual's own experiences to direct the individual's energy into the individual's ‘functional’ or ‘preferred” energy field. Energy healing encompasses a large array of slightly different therapies including but not limited to: Psychology, Healing Touch, Reflexology, Biofield Therapeutics, Therapeutic Touch, Reiki, and Chi Gung Therapy. However, none of these therapies provide a quantitative approach in both assessing an individual's energy pattern and providing direction or focus to an individual's preferred energy pattern. Such a quantitative approach is needed and the present invention supplies a needed approach.

### Terms and Definitions (In Alphabetical Order)

(Note: terms bolded and italicized in this section are also defined in this Terms and Definitions section)

Attitudinal Trait Descriptions—Description A and Description B
A plurality of a set of two descriptions where one description (Description A) is linked to its alternative or opposite description (Description B) forming a binary or opposite relationship. A description represents a user's “more likely” or “less likely” description (i.e., behavior or attitude). For example, for one description component (e.g., Acceptance) Description A could be “likes to reflect before acting” and Description B could be “takes immediate action to get things done”. As it will be described and demonstrated throughout this invention, during the intake, the user selects or chooses which descriptions from a plurality of sets of descriptions are “more-like” themselves. And by default, the opposite description, not selected by the user, becomes the description which is “less-like” the user. See Table A—Description Construct Table and refer to subsection 3 & 4 of Section A. Also refer to “charge fields” substructure1 in FIG. 2.0.0.0.

Charge Field Coordinate
The charge field coordinate identifies either the “more-like” or “less-like” users attitudinal trait descriptions. This coordinate is completely independent from the energy field coordinates defined below and it is one aspect of the relationship anatomy model (see FIG. 2.0.0.0). As defined in Table—B Discussion Generator under the charge field coordinate field and in the FIG. 2.0.0.0 thru 2.4.0.0, this coordinate is represented on the horizontal plane (e.g., left side and right side) or corresponding to the x-axis, independent from the relative bond x-axis used for the motivational coping techniques, representing either the left side which represents the “less-like” description or the right side which represents the “more-like” description (refer to FIG. 2.0.0.0.). The less like descriptions represent descriptions that are “less-like” the user. The more like descriptions represent descriptions that are “more-like” the user. During the intake, the user chooses which descriptions they are “more-like” (see attitudinal trait descriptions above in this section). Although a user may later change which descriptions are “more-like” descriptions, generally, once the intake is set the users choice of descriptions don't change very often. As shown in FIG. 2.0.0.0 the charge field substructure 1, it is equally important to note that the “more-like” and “less-like” descriptions together represent a positive and negative charge wherein one cannot exist without the other. In addition, although this “charge” is conceptual in the relationship anatomy model, it represents the constant energy force. See Table B—Discussion Generator and FIG. 2.0.0.0 through 2.4.0.0.

Combined Inquiry
For each state of mind within every fundamental category a complete inquiry is constructed by combining the energy level inquiry component (e.g., How will you), the motivational coping technique inquiry component (e.g., respect), the prefix inquiry component (e.g., -blank-, or “in”), the motivational attribute inquiry component (e.g., others strength of being), and either the more like description inquiry component or the less like description inquiry component. The more like description inquiry component (e.g., friendly and easy to know) is selected for the inquiry being generated when the charge field coordinate equals “more” and the less like description inquiry component (e.g., able to work well alone; not selected in this example) is selected for the inquiry being generated when the charge field coordinate equals “less”. (e.g., How will you respect your strength of being friendly and easy to know?). See Table B—Discussion Generator and FIG. 2.0.0.0 through 2.4.0.0.

Concatenate Energy Field Coordinates
The concatenation of each energy field coordinate consists of the three values from the energy field coordinates for each state of mind wherein the first value is the relative bond (x-axis) measure, the second value is the energyΔ (y-axis) measure, and the third value is the time (z-axis) measure. The concatenated digits from the energy field coordinates are a combination of 1, 0, or −1. A unique combination of energy field coordinates is presented for every fundamental category. Refer to measurable outcomes and See Table B—Discussion Generator and FIG. 2.0.0.0 through 2.4.0.0.

Description Choice Evaluation Procedure
For each set of descriptions, the user chooses either Description A or Description B whichever description is “more-like” themselves. The user chooses the description by entering A for Description A or B for Description B in this field. The description in which the user chooses becomes the more like description, the one that is “more-like” themselves. The other description becomes the less like description, the one that is “less-like” themselves. See Table A—Description Construct Table and FIG. 2.0.0.0 through 2.4.0.0.

Description Component
For each fundamental category (e.g., Honor) there are a set of description components. Each Description Component (e.g., Acceptance) represents or describes a set of descriptions (i.e., Description A and Description B). See Table A—Description Construct Table.

Description Constructs Table
The Description Constructs Table stores a portion of the data used (e.g., attitudinal trait descriptions) to build inquires which are generated back to the user for investigation of the user's discussion topic. It also is used during the user intake wherein the user chooses descriptions (e.g., either Description A or Description B) which best describe themselves. See Table A—Description Construct Table.

Description Inquiry Component
For each fundamental category (i.e., honor) two description inquiry components are retrieved from the description construct table, a more like description inquiry component and a less like description inquiry component. To retrieve the more like description inquiry component three fields are concatenated; the fundamental category from the discussion generator, the discussion number from the user presentation, and the fundamental category description choice from the discussion generator. Together, these fields lookup the combined value in the tripletkey field in the description construct table and retrieve the description from the more like description field. To retrieve the less like description inquiry component the same three fields are concatenated; the fundamental category from the discussion generator, the discussion number from the user presentation, and the fundamental category description choice from the discussion generator. Together, these fields lookup the combined value in the tripletkey field in the description construct table and retrieve the description from the less like description field. See Table A—Description Construct and Table B—Discussion Generator and Table C—User Presentation.

Description Type
There are two types of descriptions, independent and dependent. Independent descriptions are “stand alone” descriptions (i.e., a strength or interest description). Dependent descriptions are those that have a “linked” or “cause and effect” description (e.g., need and stress) wherein one description (e.g., need description) causes another description (i.e., stress description) to become active when the first description (e.g., need) is not satisfied. See Table A—Description Construct and Table B—Discussion Generator and Table C—User Presentation.

Discussion Generator
The Discussion Generator gathers input from the facilitator and generates for each state of mind for every fundamental category output in the form of inquires that are made available to be communicated back to the user in the user presentation. See Table B—Discussion Generator and Table C—User Presentation.

Discussion Number
A discussion number is defined as one of a set of numbers (e.g., 1 through 11) in the Table A—Description Construct Table wherein the set of numbers correspond with each set of description components (e.g., for at least one embodiment of the present invention there are 11 description components) within each fundamental category. The discussion number is either randomly generated or chosen by the user in the user presentation. At least for one embodiment of this invention, the discussion number is generated randomly on the user presentation during a users evaluation of a discussion topic. However, the user via the user presentation has the choice of executing a new random number during a discussion topic evaluation by clicking the button “next discussion”. The number generated or selected will present the user with inquiries that include the attitudinal trait descriptions that correspond to the discussion number for each fundamental category in the Table A—Description Construct Table. So, when the user changes the discussion number on the user presentation a new set of attitudinal trait descriptions are generated for each inquiry on the user presentation. See Table A—Description Construct Table and Table B—Discussion Generator and Table C—User Presentation and FIG. 3.0.0.0.

### Discussion Topic

A discussion topic is selected by the user wherein the discussion topic is the subject in which the user would like to investigate during a user's evaluation of a discussion topic. The discussion topics defined are Family, Friends, Faith, Finances, Work, and Home. The discussion topics are general life areas wherein the user may select a general discussion topic to investigate more specific situations. For example, a user may select “work” in a situation where the user needs to prepare for an important meeting. See Table C—User Presentation.

### Discussion Topic Evaluation Statements

The discussion topic evaluation statements evaluate or measure how a user perceives the discussion topic in which the user wishes to investigate. The discussion topic evaluation consists of three statements that correspond directly to each of the three axis defined in the discussion generator in the energy field coordinates (i.e., relative bond (x-axis), energyΔ (y-axis), and time (z-axis)). In the following example the user is assumed to have chosen “work” as the discussion topic. Also, the statements in the discussion topic evaluation are designed to have no right or wrong answers, only expect the user to answer the statements in regard to how they truthfully feel or think. The first statement (e.g., a)) corresponding to the relative bond (x-axis) axis is “I feel stable when it comes to my work” wherein the meaning of the word “stable” may communicate a state of mind where the user is secure or not secure toward issues regarding the discussion topic. The answers for the first statement are either “yes” or “no” wherein “yes” equals 1 which represents a “repulsive” or extrinsic-oriented motivational coping technique and “no” equals 0 which represents an “attraction” or intrinsic-oriented motivational coping technique. The second statement (e.g., b)) corresponding to the energy (y-axis) axis is “I feel enthusiastic when it comes to my work” wherein the meaning of the word “enthusiastic” communicates the user's level of energy toward the discussion topic. The answers for the second statement are either “yes” or “no” wherein “yes” equals 1 which represents an “active-oriented” (e.g., initiating more effort) motivational coping technique and “no” equals 0 which represents a “passive-oriented” (e.g., initiating less effort) motivational coping technique. And the third statement (e.g., c)) corresponding to the time (z-axis) axis is “I feel confident when it comes to my work” wherein the word “confident” establishes a timeframe reference with the discussion topic as to relate the inquiries which are generated back to the user toward a state of mind which the user may optimize their investigation of the discussion topic. The answers for the third statement are either “yes”, “no” or “used to be” wherein “yes” equals 1 which relates to future time, “no” equals 0 which relates to present time, and “used to be” equals −1 which relates to past time. See Table B—Discussion Generator and Table C—User Presentation and FIG. 2.0.0.0 through 2.4.0.0.

### Discussion Topic Inquiries

The discussion topic inquiries are generated inquiries derived from the user's answers from the discussion topic evaluation statements. For at least one embodiment described in this invention, four inquiries are derived, one for each fundamental category. However, more inquires may be generated if additional fundamental categories are defined. First, the numbers derived from the users answers in the discussion topic evaluation statements for any combination of “yes” (1) or “no” (0) or “used to be” (—1) are concatenated (e.g., 110 wherein the first 1 is from statement a), the second 1 is from statement b) and 0 is from statement c)). The users concatenated answers (e.g., 110) are used as a key to retrieve the combined inquiry in the concatenate energy field coordinates field which is derived from the energy field coordinates from the discussion generator for each fundamental category. Refer to discussion topic evaluation and See Table B—Discussion Generator and Table C—User Presentation.

### Doublekey

This is a key field that is a concatenation of the fundamental category and the discussion number. This field is used in the discussion generator under the field called the fundamental category description choice to lookup the user's chosen “more-like” themselves attitudinal trait description choice in the description construct table under the field called the description choice evaluation procedure. See Table A—Description Construct Table and Table B—Discussion Generator.
   EnergyΔ (y-axis)
Energy represents the y-axis in the relationship anatomy model wherein there are at least two possible outcomes, 1 or 0, wherein 1 represents the top of the y-axis labeled as Δ₂ energy and the 0 represents the bottom of the y-axis labeled as Δ₁ energy. The lower Δ₁ energy level represent motivational coping techniques which are “passive-oriented” wherein “passive-oriented” motivational coping techniques are designed to convey less effort and are considered “beginning-oriented” motivational coping techniques (e.g., “allow”). In addition, regarding the development of inquiries wherein the user studies these inquiries to investigate a particular discussion topic, a portion of the inquiry, at least in one embodiment of the present invention, is developed based on the two energy levels wherein the lower Δ₁ energy is regarded as a more passive oriented inquiry that begins with “What” which assumes a user to be more inactive in their pursuit to discover answers in their investigation of the discussion topic. On the other hand, the higher Δ₂ energy level represent motivational coping techniques which are “active-oriented” wherein “active-oriented” motivational coping techniques are designed to convey more effort and are considered “advanced-oriented” motivational coping techniques (e.g., “accept”). In addition, the higher Δ₂ energy is regarded as a more active oriented inquiry that begins with “How” which assumes a user to be more active in their pursuit to discover answers in their investigation of the discussion topic. See Table B—Discussion Generator (specifically the energy level inquiry component) and FIG. 2.0.0.0 through 2.4.0.0.

### Energy Field Coordinates

Refer to State of Mind.

### Energy Level Inquiry Component

The energy level inquiry component is a portion of the inquiry that is generated back to the user based on the users evaluation of a discussion topic from the user presentation. This particular component is the beginning part of the inquiry. The beginning part of the inquiry is defined for every state of mind in each fundamental category based on the position related to the time axis (i.e., z-axis) and the energy axis (i.e., y-axis). It is defined as “What do you” when the position on the time axis (i.e., z-axis) is 0 (i.e., “do” represents present time) and the position of the energy axis (i.e., y-axis) is 0 (i.e., Δ₁ energy: see energyΔ (y)). It is defined as “How do you” when the position on the time axis (i.e., z-axis) is 0 (i.e., present time) and the position of the energy axis (i.e., y-axis) is 1 (i.e., Δ₂ energy: see energyΔ (y)). It is defined as “What will you” when the position on the time axis (i.e., z-axis) is 1 (i.e., “will” represents future time) and the position of the energy axis (i.e., y-axis) is 0 (i.e., Δ₁ energy: see energyΔ (y)). It is defined as “How will you” when the position on the time axis (i.e., z-axis) is 1 (i.e., future time) and the position of the energy axis (i.e., y-axis) is 1 (i.e., Δ₂ energy: see energyΔ (y)). It is defined as “What did you” when the position on the time axis (i.e., z-axis) is −1 (i.e., “did” represents past time) and the position of the energy axis (i.e., y-axis) is 0 (i.e., Δ₁ energy: see energyΔ (y)). It is defined as “How did you” when the position on the time axis (i.e., z-axis) is −1 (i.e., past time) and the position of the energy axis (i.e., y-axis) is 1 (i.e., Δ₂ energy: see energyΔ (y)). See Table B—Discussion Generator and FIG. 2.0.0.0 through 2.4.0.0.

### Fundamental Category

A fundamental category is an essential quality which represents one of multiple “ideal” ways to relate to a person, thing, or situation (In one embodiment, fundamental categories are defined as “honor”, “devotion”, “confidence”, and “patience” wherein when such fundamentals work together are intended to represent an optimum relationship. See Table A—Description Construct and Table B—Discussion Generator and FIG. 2.0.0.0 through 2.4.0.0.

### Fundamental Category Description Choice

This field concatenates the related fundamental category and the discussion number from the user presentation and looks up this value in the doublekey field from the Table A—Descriptions Construct Table and retrieves the description choice evaluation procedure for that related fundamental category, either a or b. See Table A—Description Construct and Table B—Discussion Generator.
   Less like Description
For each set of descriptions, this is the description (behavior or attitude), either Description A or Description B, that least represents the user (i.e., less-like the user). These resemble descriptions from the environment or others in which they are not familiar with or feel unnatural doing themselves. See Table A—Description Construct Table.
   Less like Description Inquiry Component
The less like description component is a portion of the combined inquiry. This component is the description (behavior or attitude), either Description A or Description B, that least represents the user (i.e., less-like the user). This description represents the opposite description (either Description A or Description B) from the set of descriptions wherein this description component is the description from which the user does not select to be more-like themselves. See Table B—Discussion Generator.
   More like Description
For each set of descriptions, this is the description (behavior or attitude), either Description A or Description B, that most represents the user (i.e., more-like the user). These resemble descriptions wherein the user is familiar with or feels natural doing themselves. See Table A—Description Construct Table.
   More like Description Inquiry Component
The less like description component is a portion of the combined inquiry. This is the description (behavior or attitude), either Description A or Description B, that most represents the user (i.e., more-like the user). This description was selected or chosen by the user to be more-like themselves. See Table B—Discussion Generator.

### Motivational Attribute

A motivational attribute (e.g., “need”) is an essential source or driver which serves to develop or evolve a fundamental category (e.g., “confidence”). Each fundamental category defines at least one motivational attribute and the motivational attribute is incorporated into the inquiries generated back to the user from the discussion generator under the field called the motivational attribute inquiry component. In addition, each motivational attribute (e.g., “need”) classifies a set of attitudinal trait descriptions (e.g., “plenty of time to make complex decisions”) for each fundamental category. See Table A—Description Construct Table.

### Motivational Attribute Inquiry Component

For each fundamental category (e.g., “confidence”) a different motivational attribute is defined (i.e., “need”). The motivational attribute inquiry component is a part of the inquiry presented back to the user in the user presentation. There are two parts contained in this piece of the inquiry. The first part, as just described above, is the motivational attribute that has a one-to-one relationship to the fundamental category (e.g., “need” is the motivational attribute for the fundamental category “confidence”). The second part of this piece of the inquiry directs the user to focus either on themselves or others. Under the x-axis of the charge field coordinate field in Table B—Discussion Generator, represents a piece of the inquiry component that will generate an inquiry focused on “you”, the user, when the value is “more” (refer to more like descriptions) and this component will generate an inquiry focused on “others” when the value is “less” (refer to less like descriptions). Specifically, for the fundamental category “honor”, when the charge field coordinate is “more” the motivational attribute inquiry component is “your strength of being”, otherwise when the charge field coordinate is “less” the motivational attribute inquiry component is “others strength of being” wherein both cases the motivational attribute is “strength”. For the fundamental category “devotion”, when the charge field coordinate is “more” the motivational attribute inquiry component is “your interest in”, otherwise when the charge field coordinate is “less” the motivational attribute inquiry component is “others interest in” wherein both cases the motivational attribute is “interest”. For the fundamental category “confidence”, when the charge field coordinate is “more” the motivational attribute inquiry component is “your need for”, otherwise when the charge field coordinate is “less” the motivational attribute inquiry component is “others need for” wherein both cases the motivational attribute is “need”. For the fundamental category “patience”, when the charge field coordinate is “more” the motivational attribute inquiry component is “your stress toward”, otherwise when the charge field coordinate is “less” the motivational attribute inquiry component is “others stress toward” wherein both cases the motivational attribute is “stress”. See Table B—Discussion Generator and FIG. 2.0.0.0 through 2.4.0.0.

### Motivational Coping Technique

A motivational coping technique (e.g., “allow”) is a verb (e.g., this verb must also have a capability to be used as a transitive verb) representing a motive which is intended to develop or evolve a user's learning of each fundamental category (e.g., confidence). At least in one embodiment of this invention there are at least four motivational coping techniques defined for every fundamental category. Referring to FIG. 2.0.0.0 through 2.4.0.0., motivational coping techniques are designed in the relationship anatomy model for each quadrant cache. As described by the four rules for defining motivational coping techniques in subsection 9 of Section A in the Detailed Description of the Invention, Quadrant 1 Cache represents motivational coping techniques that are “passive-oriented” which convey less effort or “beginning-oriented” techniques and represents motivational coping techniques that convey a “repulsive” or extrinsic-oriented meaning (e.g., “support” is assumed to convey an extrinsic action). Quadrant 2 Cache represents motivational coping techniques that are “passive-oriented” which convey less effort or “beginning-oriented” techniques and represents motivational coping techniques that convey an “attraction” or intrinsic-oriented meaning (e.g., “allow” is assumed to convey an intrinsic action). Quadrant 3 Cache represents motivational coping techniques that are “active-oriented” which convey more effort or “advanced-oriented” techniques and represents motivational coping techniques that convey a “repulsive” or extrinsic-oriented meaning (e.g., “maintain” is assumed to convey an extrinsic action). Quadrant 4 Cache represents motivational coping techniques that are “active-oriented” which convey more effort or “advanced-oriented” techniques and represents motivational coping techniques that convey an “attraction” or intrinsic-oriented meaning (e.g., “accept” is assumed to convey an intrinsic action). See Table B—Discussion Generator and FIG. 2.0.0.0 through 2.4.0.0 and refer to subsection 9 of Section A in the Detailed Description of the Invention.

### Motivational Coping Technique Inquiry Component

For each fundamental category a set of motivational coping techniques are defined and presented in a particular order in the relationship anatomy model. The motivational coping technique inquiry component is a field defined in the discussion generator that represents a portion of the combined inquiry that is generated back to the user based on the user's input from the user presentation. The motivational coping techniques are defined based on the design of the relationship anatomy model. See Motivational Coping Technique and Table B—Discussion Generator and FIG. 2.0.0.0 through 2.4.0.0.

### Prefix Inquiry Component

The prefix inquiry component is a portion of the combined inquiry that is generated back to the user in the user presentation. The prefix inquiry component “in” is presented after the motivational coping technique inquiry component and prior to the motivational attribute inquiry component in the discussion generator. The prefix “in” is only presented in the inquiry back to the user in the user presentation when the energy field coordinate in the y-axis value is 0 (this represents the bottom of the y-axis or lower energy), otherwise, it is left blank. See Table B—Discussion Generator.

### Quadrant Cache

A quadrant cache is part of the Relationship Anatomy Model design. A quadrant cache represents a plurality of interchangeable variables including, at least for one embodiment of this invention, the user's descriptions and the motivational coping techniques. First, referring to FIG. 2.0.0.0 through 2.4.0.0 for the remainder of this description, quadrant caches are divided into, at least in one embodiment of this invention, four quadrant caches by two axes, the relative bond x-axis and the energy y-axis. One side of the relative bond x-axis represents motivational coping techniques that convey a “repulsive” or extrinsic-oriented meaning (e.g., “support”) and the other side of the relative bond x-axis represents motivational coping techniques that convey an “attraction” or intrinsic-oriented meaning (e.g., “allow”). The second axis is called the energy y-axis wherein the top of the y-axis represents motivational coping techniques that convey an “active-oriented” motivational coping technique wherein it is assumed that a user is generally initiating activities to address the high energy/active instance. In addition, “active-oriented” motivational coping techniques are “advanced-oriented” techniques (e.g., “accept”) compared to “passive-oriented” motivational coping techniques (e.g., “allow”). The bottom of the energy y-axis represents motivational coping techniques that convey lower energy which are considered “passive-oriented” motivational coping techniques wherein it is assumed that a user is generally not initiating activities but rather contemplate ways to address the low energy/inactive instance. In addition, “passive-oriented” motivational coping techniques are “beginning-oriented” techniques (e.g., “allow”) compared to “active-oriented” motivational coping techniques (e.g., “accept”). There are four quadrant caches represented by the two axes in the relationship anatomy model for each fundamental category. Quadrant 1 Cache represents motivational coping techniques that are “passive-oriented” which convey less effort or “beginning-oriented” techniques and represents motivational coping techniques that convey a “repulsive” or extrinsic-oriented meaning (e.g., “support”). In addition, Quadrant 1 Cache represents a users “more-like” attitudinal trait descriptions. Quadrant 2 Cache represents motivational coping techniques that are “passive-oriented” which convey less effort or “beginning-oriented” techniques and represents motivational coping techniques that convey an “attraction” or intrinsic-oriented meaning (e.g., “allow”). In addition, Quadrant 2 Cache represents a user's “less-like” attitudinal trait descriptions. Quadrant 3 Cache represents motivational coping techniques that are “active-oriented” which convey more effort or “advanced-oriented” techniques and represents motivational coping techniques that convey a “repulsive” or extrinsic-oriented meaning (e.g., “maintain”). In addition, Quadrant 3 Cache represents a users “more-like” attitudinal trait descriptions. Quadrant 4 Cache represents motivational coping techniques that are “active-oriented” which convey more effort or “advanced-oriented” techniques and represents motivational coping techniques that convey an “attraction” or intrinsic-oriented meaning (e.g., “accept”). In addition, Quadrant 4 Cache represents a user's “less-like” attitudinal trait descriptions. See FIG. 2.0.0.0 through 2.4.0.0 and refer to subsection 8a of Section A in the Detailed Description of the Invention.

### Relationship Anatomy Model

Referring to FIG. 2.0.0.0 through 2.4.0.0., the relationship anatomy model is designed to learn fundamental categories with low to no resistance (see Supplemental A—Design of the Relationship Anatomy Model (Energy in Superconductors)). The relationship anatomy model is a visual representation that presents how a user relates their perception of a user-defined situation (e.g., discussion topic) to the data model's predefined data organization (i.e., motivational coping technique inquiry component structure defined in the discussion generator), wherein this data organization includes related or “linked” data items, each data item identifying/describing an attribute, characteristic, or classification (e.g., defined in the quadrant caches) of the user's motivations and/or personal traits (i.e., the more-like descriptions and less-like descriptions). Moreover, this data organization may be used by the user to investigate his/her perceptions regarding various discussion topics. In particular, each such data item can be from one (and only one) of the following components or classifications of the users motivations and/or personal traits: fundamental categories, user states of mind, user motivational attributes, user motivational coping techniques, and the user's attitudinal trait descriptions (See attitudinal trait description A and description B). Accordingly, the relationship anatomy model's components or classification are used to generate meaningful inquiries or questions to the user through the discussion generator and present it to the user in the user presentation. The discussion generator is designed based on the relationship anatomy model wherein the discussion generator constructs the inquiries using the description construct table and the user presentation presents inquiries back to the user based on the users perception of the discussion topic, wherein these inquiries are constructed directly from the linkages of the data items of the user's specific relationship anatomy model. In addition, the description constructs table is designed based on the data organization of the relationship anatomy model. See Table A—Description Construct Table and Table B—Discussion Generator and FIG. 2.0.0.0 thru 2.4.0.0.
   Relative Bond (x-axis)
Relative bond is an aspect of the energy field coordinates that represents the x-axis wherein there are at least two possible outcomes, 1 or 0, wherein 1 represents “repulsive” or extrinsic-oriented motivational coping techniques (e.g., “support”) and 0 represents “attraction” or intrinsic-oriented motivational coping techniques (e.g., “allow”) See Table B—Discussion Generator and FIG. 2.0.0.0 through 2.4.0.0 and refer to subsection 6 of Section A in the Detailed Description of the Invention.

### Set of Descriptions

See Description A and Description B above in this section. See Table A—Description Construct Table.

### State of Mind

For each fundamental category, a combination of three different values defined under the energy field coordinates field in Table B—Discussion Generator represents at least twelve different user's states of mind outcomes, denoted as A through L in the state of mind field located in the Table B—Discussion Generator. It is important to note that the energy field coordinates which are directly related to the motivational coping techniques are completely independent from the charge field coordinate which is directly related to the attitudinal trait descriptions (see charge field coordinate above in this section). The first value of the energy field coordinates, relative bond, represents the x-axis wherein there are at least two possible outcomes, 1 or 0, wherein 1 represents “repulsive” motivational coping techniques having an extrinsic-oriented meaning (e.g., “support” is assumed to convey an extrinsic action) and 0 represents “attraction” motivational coping techniques having an intrinsic-oriented meaning (e.g., “allow” is assumed to convey an intrinsic action). The second value, energy, represents the y-axis wherein there are at least two possible outcomes, 1 or 0, wherein 1 represents the top of the y-axis labeled as “active-oriented” or Δ₂ energy (e.g., higher energy) and the 0 represents the bottom of the y-axis labeled as “passive-oriented” or Δ₁ energy (e.g., lower energy). The “active-oriented” or higher energy position on the y-axis represents motivational coping techniques that are considered “advanced-oriented” motivational coping techniques that assume a user is generally initiating activities to address the high energy/active instances and the “passive-oriented” or lower energy position on the y-axis represents motivational coping techniques that are considered “beginning-oriented” motivational coping techniques that assume a user is generally NOT initiating activities to address the low energy/inactive instance. And the third value, time, represents the z-axis wherein there are at least three possible outcomes, 1 or 0 or −1, wherein 1 represents the future time, 0 represents the present time, and −1 represents the past time. These states of mind are designed into the relationship anatomy model and provide a means of relating the user's perception to a discussion topic. See Table B—Discussion Generator and FIG. 2.0.0.0 through 2.4.0.0 and refer to subsection 10 of Section A in the Detailed Description of the Invention.

### Time (z)

Time represents the z-axis wherein there are at least three possible outcomes, 1 or 0 or −1, wherein 1 represents the future time, 0 represents the present time, and −1 represents the past time. See Table B—Discussion Generator and FIG. 2.0.0.0 through 2.4.0.0 and refer to subsection 7 of Section A in the Detailed Description of the Invention.

### Tripletkey

This is a key field that is a concatenation of the fundamental category, the discussion number, and the description choice evaluation procedure fields in the Table A—Description Construct Table. This field is used to identify the attitudinal trait descriptions used to construct inquires generated in the Table B—Discussion Generator. Table A—Description Construct Table.

### Wave Function |ψ>

For each state of mind, the concatenate energy field coordinates are converted into a wave function and represented in the relationship anatomy model for every quadrant cache position. For each fundamental category, a unique wave function exists for every state of mind. See Table B—Discussion Generator and FIG. 2.0.0.0 through 2.4.0.0 and refer to subsection 8b of Section A in the Detailed Description of the Invention.

### SUMMARY OF THE INVENTION

(Note: words shown in bold text below are defined in the Terms and Definitions section above; only the first instance of each term is in bold format)

Overall, it is important to note that the present invention provides a computational structure for creating applications wherein the structure provides a programmatic and syntactical computational framework for being able to describe or define specific applications that generate meaningful or purposeful discussions or thought processes for users to manage or learn preferred outcomes in which the application is designed or intended. For example, the computational structure of the present invention is like a “Microsoft Excel” program whereas each such application is like a “spreadsheet” created from the “Microsoft Excel” program to manage, e.g., a specific financial outcome. As a reader of the present invention, you will notice that present invention describes: (1) the computational structure along with (2) at least one application using the computational structure. In particular, the application described herein aids users in gaining greater insight or awareness for understanding: (a) their motivations, and/or (b) their likely (denoted as “more like” herein) or unlikely (denoted as “less like” herein) behavior responses toward a specific user-defined situation (denoted as “discussion topic” herein).

In one embodiment the present invention may be embodied as a description construct table and a discussion generator for assisting a person (i.e., a user) in becoming more aware or gaining greater understanding of his/her perceptions on various “discussion topics”, wherein a discussion topic may be a particular situation regarding, but not limited to, family, friends, faith, finances, work, home, etc.

More specifically, the present invention provides a syntactic representation (i.e., the combination of both data and programmatic algorithms) of a user's motivations and/or “more like” or “less like” behavior responses so that the syntactic representation can be used to generate customized inquiries to be provided to the user. Additionally, the syntactic representation is intended to model how a user prefers to: (a) use language to think, and (b) describe how a user prefers to perceive his/her world/environment. Thus, by combining both the data defined for a specific application with the programmatic algorithms of the present invention, a programmatic system that expresses ideas, questions and statements in language (i.e., words, terms and phrases) that is more meaningful to the user, and thereby provides the user with greater insight into how the user experiences and/or prefers to perceive his/her environment.

In one embodiment, the present invention may be considered an intelligent process for providing users with the computational structure to aid them in describing and/or assessing personalized content (e.g., exploring a particular topic) representative of preferred: (1) sets of values (denoted as “fundamental categories” herein) wherein each fundamental category has an associated motive (denoted as “motivational attribute” herein), (2) coping descriptors (i.e., descriptions of coping behavior, also denoted as “motivational coping techniques” herein), and (3) personalized attitudes or behaviors (denoted as “attitudinal trait description(s) A and description(s) B” herein). For example, for a set of values including a fundamental category denoted: “generosity”, a corresponding coping descriptor that is meaningful to the user (although not necessarily practiced by the user) might be the verb phrase: “feeding the poor”, and a set of corresponding attitudinal trait descriptions applicable to the user might be: “prefers to be with people”, and “prefers to be in control of situations”. In particular, the resulting described personalized content provides data for generating inquiries back to the user that are intended to help the user learn how to better understand, perceive and/or recognize his/her attitudes and/or perceptions as they relate to a user-defined discussion topic. Accordingly, the user can investigate, reflect upon, and/or gain greater insight into his/her perceptions related to the discussion topic and the user's specific attitudinal trait descriptions related thereto.

The discussion generator of the present invention may extract specific user attitudinal trait descriptions from a description choice evaluation procedure in the description construct table that evaluates the user's perceptions in relation to a predetermined set of attitudinal traits. Such attitudinal traits (and more particularly, the attitudinal trait descriptions derived therefrom) are then passed to the discussion generator wherein inquiries are generated to and/or interactions are initiated with the user in a user presentation so that the user gains a greater awareness of a given discussion topic. In particular, the output from such an evaluation procedure may assist the description construct table of the present invention (and the user as well) in classifying the user's “more like” (e.g., “like” behaviors or attitudes) and “less like” (e.g., “unlike” behaviors or attitudes) sets of motivational attributes so that corresponding sets of attitudinal trait descriptions can be used in generating inquiries (e.g., statements and/or questions for the user's consideration or contemplation). Accordingly, such attitudinal trait descriptions and the inquiries generated therefrom support the user in understanding, perceiving, and/or recognizing his/her or other's behaviors or attitudes related to a discussion topic.

Furthermore, in generating such inquiries, the invention uses (i) a pre-determined set of fundamental categories (e.g., in one embodiment: “honor”, “devotion”, “confidence”, “patience”) that represent “ideal” ways a user may relate to themselves or interact with their environment, and (ii) corresponding motivational coping techniques (e.g., in one embodiment for the fundamental category “confidence”: “allow”, “accept”, “support”, “maintain”) that helps a user develop or evolve an awareness of each of the fundamental categories as they relate to a discussion topic. Note that such fundamental categories may be predefined by a user along with a “facilitator” who utilizes the present invention to aid the user in discovering ways to learn and/or cope with their “more like” and “less like” sets of attitudinal trait descriptions (e.g., behaviors or attitudes) during a process of associating and/or learning ways to incorporate personally important characteristics of such fundamental categories (e.g., “confidence”) into the user's awareness. In particular, it is an aspect of the present invention that a facilitator may utilize this invention to aid the user in discovering ways to cope (e.g., “allow”) with their “more like” and “less like” sets of attitudinal trait descriptions (e.g., needing more time to make decisions) in the context of a discussion topic defined by the user (e.g., work).

Moreover, it is yet another aspect of the present invention that the predetermined fundamental categories and their associated motivational attributes, the motivational coping techniques, and the sets of attitudinal trait descriptions are designed in such a manner that a user may input evaluations of a discussion topic into the invention, and the user presentation through the discussion generator of the present invention will use such evaluation for communicating back to the user through carefully designed inquiries which are the result of computational analysis of the evaluations as they relate to such fundamental categories and their associated motivational attributes, motivational coping techniques and sets of attitudinal trait descriptions so that the user can then consider (and preferably answer or address) subsequent statements or questions generated by the invention, and thereby gain a greater understanding of his/her motivations related to the discussion topic.

An additional aspect of the present invention is directed to a computational system (method and apparatus) for generating such discussion-topic directed inquiries and/or discussion-topic related interactions according to a predetermined set of one or more fundamental categories as defined by the facilitator and/or the user. In a typical embodiment of the invention, each such fundamental category represents a healthy quality (e.g., “confidence”) of how the user may wish to perceive issues, situations and/or relationships. In addition, the facilitator and the user are able to define a set of motivational coping techniques for each fundamental category designed accordingly through what is referred to herein as the Relationship Anatomy Model that is particularly disclosed in FIG. 2.0.0.0 thru 2.4.0.0 as it relates to the and refer to subsections 8 and 9 of Section A in the Detailed Description Of The Invention. The Relationship Anatomy Model is a structure made of substructures wherein these substructures are designed to imitate or emulate how personal energy or thought can be optimally managed. Furthermore, the design of this entire computational system is based on the Relationship Anatomy Model structure shown in FIG. 2.0.0.0 through 2.4.0.0. More specifically, the Relationship Anatomy Model is designed in substructures wherein these substructures are designed to model different classifications of the fundamental categories, motivational attributes, motivational coping techniques, and the attitudinal trait descriptions. These substructures and their classifications are combined into one dynamic structure, the Relationship Anatomy Model, wherein this structure contains a process to generate inquiries back to the user based on the users evaluation of a discussion topic that is carefully designed to provide a user of the system a meaningful way to address or approach personal discussion topics with the intention of improving a user's outlook toward such discussion topics.

The motivational coping techniques (see FIG. 2.1.0.0 through 2.4.0.0) are identified or represented as verbs in the present invention, wherein such verbs are specifically selected with the intention of developing or evolving awareness by the user of the fundamental category for which the verbs are representative. Each motivational coping technique is defined through specific requirements of the system as described in subsection 9 of Section A in the Detailed Description of the Invention. The motivational coping techniques are carefully designed to relate, through inquiries generated back to the user, a preferred way to address or approach a users more like or less like attitudinal trait descriptions in regards to a user's discussion topic evaluation. As described above, a qualified facilitator or one skilled in the art may be required to predefine every fundamental category and the corresponding motivational coping techniques. Also as described above, attitudinal trait descriptions are classified into motivational attributes. Each such motivational attribute (e.g., “strength”, “interests”, “needs”, “stress”) is also classified in Table A—Description Construct Table within a fundamental category (e.g., “honor”, “devotion”, “confidence”, “patience”). The relationships between fundamental categories, motivational attributes, motivational coping techniques, and attitudinal trait descriptions are more apparent in Section A in the Detailed Description of the Invention.

### BRIEF DESCRIPTION OF THE DRAWINGS

FIG. 1.0.0.0 shows a TOP LEVEL block diagram of the invention.

FIG. 1.1.0.0 shows a block diagram of the base-component called A—DESCRIPTION CONSTRUCT TABLE 1. This component utilizes the facilitator to classify the fundamental categories, the fundamental categories associated motivational attributes and the attitudinal trait descriptions (e.g., behaviors or attitudes) that are made available to the user for choosing which attitudinal trait descriptions are “more-like” and “less-like” themselves.

FIG. 1.1.1.0 shows a block diagram of the sub-component called USER DESCRIPTION CHOICE 11. This sub-component utilizes the user to describe themselves by choosing or classifying attitudinal trait descriptions (e.g., behaviors or attitudes) that are “more-like” themselves. Each classification includes two descriptions for the user to choose. Only one of the descriptions can be chosen to be “more-like” themselves. Once a selection is made, the description that is not chosen is classified to be “less-like” themselves. This sub-component is from the base-component called A—DESCRIPTION CONSTRUCT TABLE 1 described above.

FIG. 1.2.0.0 shows a block diagram of the base-component called DISCUSSION GENERATOR 2. This component utilizes the design of the relationship anatomy model to classify the states of mind for every fundamental category by defining energy field coordinates (i.e., wave functions), constructs and stores the motivational coping techniques for every fundamental category, and designs and generates inquires for every state of mind based on a plurality of variables.

FIG. 1.2.1.0 shows a block diagram of the sub-component called MOTIVATIONAL COPING TECHNIQUE CONSTRUCTOR 24. This component utilizes the facilitator and the motivational coping technique rules to define each of the motivational coping techniques for every fundamental category. This sub-component is from the base-component called DISCUSSION GENERATOR 2 described above.

FIG. 1.2.2.0 shows a block diagram of the sub-component called COMBINED INQUIRY 30. This sub-component finds and classifies the users “more-like” and “less-like” attitudinal trait descriptions for each fundamental category and at least partially based on the discussion number defined from a user's discussion topic evaluation. These attitudinal trait descriptions are used in the design to construct the inquiries for each state of mind. This component is from the base-component called DISCUSSION GENERATOR 2 described above.

FIG. 1.2.2.1 shows a block diagram of the sub-component called DESCRIPTION INQUIRY COMPONENT 35. This sub-component finds and classifies the users “more-like” and “less-like” attitudinal trait descriptions for each fundamental category and at least partially based on the discussion number defined from a user's discussion topic evaluation. These attitudinal trait descriptions are used in the design to construct the inquiries for each state of mind. This sub-component is from the sub-component called COMBINED INQUIRY 30 described above.

FIG. 1.3.0.0 shows a block diagram of the base-component called C—USER PRESENTATION 3. This component utilizes the user in defining a discussion topic, performing a discussion topic evaluation by answering three discussion topic evaluation statements and presents the most optimal inquiries for each fundamental category for the user to use in their investigation of the discussion topic.

FIG. 1.3.1.0 shows a block diagram of the sub-component called GENERATE DISCUSSION TOPIC INQUIRIES 54. This sub-component retrieves the most optimal inquiries from the discussion generator based on the user's evaluation of a discussion topic. This sub-component is from the base-component called C—USER PRESENTATION 3 described above.

FIG. 2.0.0.0 shows the substructures that when combined together represent a relationship anatomy model module. The first substructure, substructure 1, classifies the attitudinal trait descriptions into a user's “more-like” and “less-like” attitudinal trait descriptions called charge fields. The second substructure, substructure 2, classifies the motivational coping techniques into “passive-oriented” and “active-oriented” energy levels called energy fields. The third substructure, substructure 3, classifies motivational coping techniques into four quadrant caches with an x-axis called relative bond and a y-axis called energy. The x-axis represents motivational coping techniques as either “repulsive” (e.g., a push type of energy) and “attraction” (e.g., a pull type of energy). The y-axis represents motivational coping techniques as either “high” or “low” energy. The fourth substructure, substructure 4, shows the z-axis called time wherein relationship anatomy model modules are classified into different time references, present time, future time, and past time (this is more apparent in the figures described immediately below).

FIG. 2.1.0.0 shows the relationship anatomy model modules for each time reference (e.g., present, future, and past time), a classification of the motivational coping techniques for the fundamental category “honor”, and the classification of the energy field coordinates or wave functions for each state of mind for a fundamental category.

FIG. 2.2.0.0 shows the relationship anatomy model modules for each time reference (e.g., present, future, and past time), a classification of the motivational coping techniques for the fundamental category “devotion”, and the classification of the energy field coordinates or wave functions for each state of mind for a fundamental category.

FIG. 2.3.0.0 shows the relationship anatomy model modules for each time reference (e.g., present, future, and past time), a classification of the motivational coping techniques for the fundamental category “confidence”, and the classification of the energy field coordinates or wave functions for each state of mind for a fundamental category.

FIG. 2.4.0.0 shows the relationship anatomy model modules for each time reference (e.g., present, future, and past time), a classification of the motivational coping techniques for the fundamental category “patience”, and the classification of the energy field coordinates or wave functions for each state of mind for a fundamental category.

FIG. 3.0.0.0 shows the users interface or presentation of a discussion topic evaluation. This is where the user defines a discussion topic and answers three discussion topic evaluation statements regarding the discussion topic. Once the three discussion topic evaluation statements are answered a series of inquiries, one for each fundamental category, is presented to the user based on the answers to the discussion topic evaluation statements wherein these inquiries are generated from the discussion generator. The user investigates the inquiries wherein the inquiries are intended to evoke ways to improve discussion topic related issues. In addition, the user may click a button called “next discussion” which changes the attitudinal trait descriptions built into the inquiries that are generated from the discussion generator. This enables a user to generate alternate inquiries to increase the possibility of discovering the optimal solutions toward helping discussion topic issues.

Table A—Description Construct Table—This Table shows all the available attitudinal trait descriptions (e.g., Description A and Description B) defined by the facilitator for each fundamental category and description component. As described as the “intake” process, this Table is also used by the user to classify the attitudinal trait descriptions as “more-like” and “less-like” themselves for each fundamental category and description component. Even though a user may classify the attitudinal trait descriptions on their own, it is encouraged to work with a qualified facilitator of the invention to help them through the intake process.

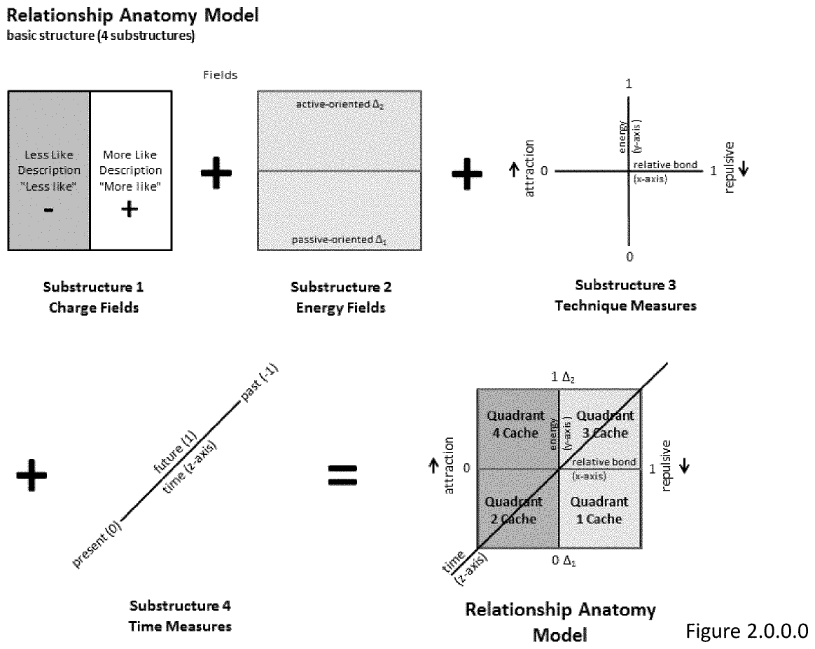

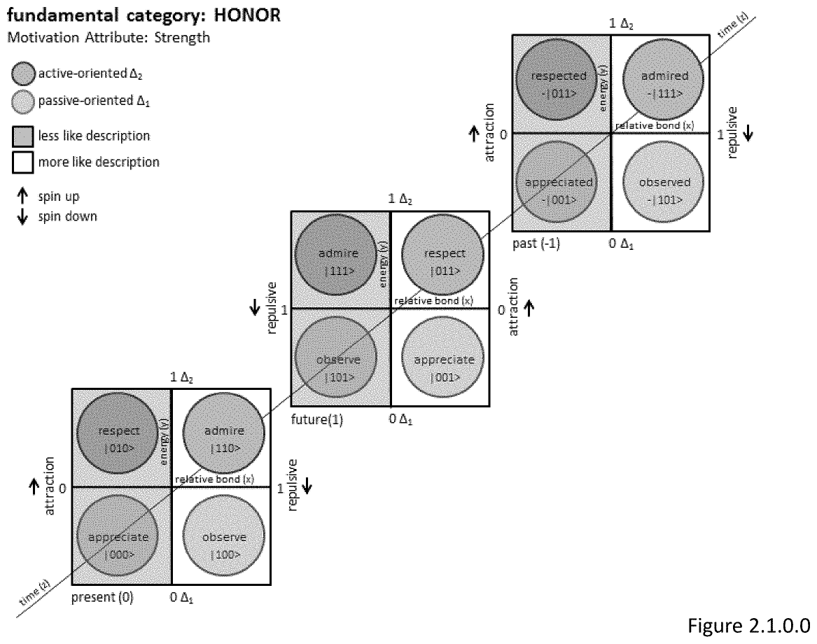

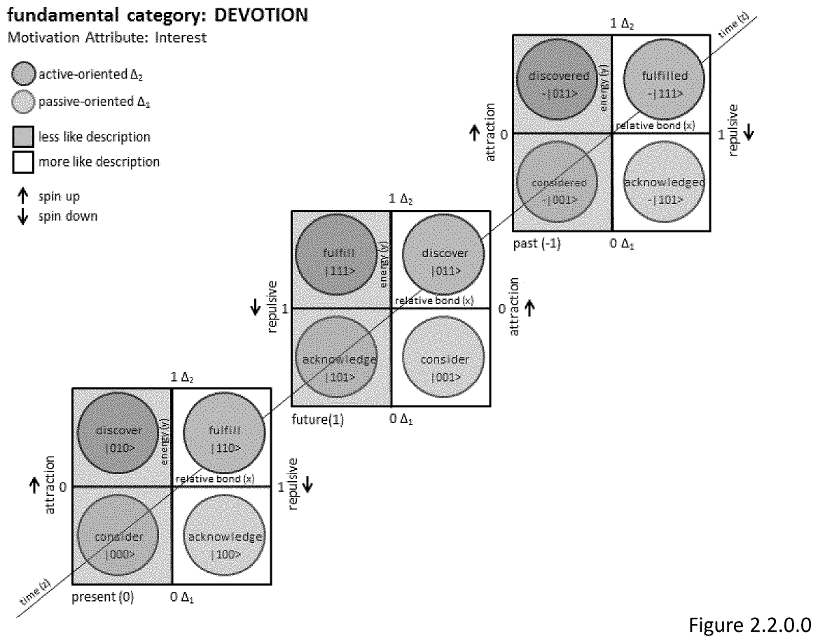

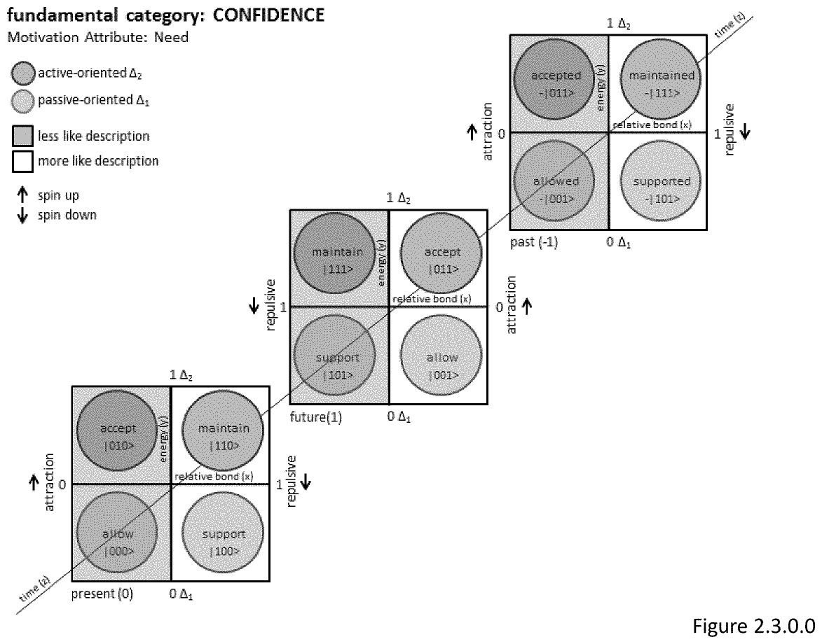

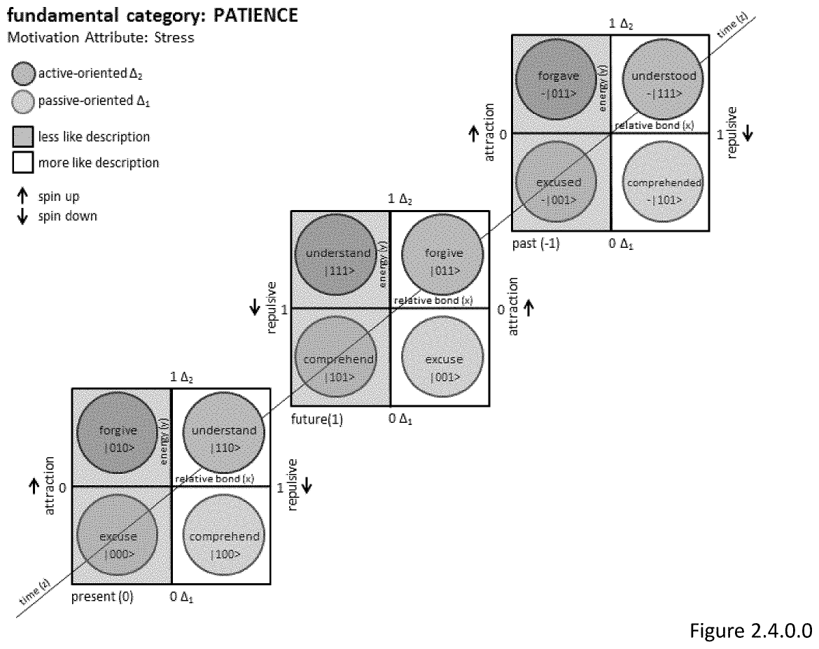

Table B—Discussion Generator—The facilitator utilizes the design of the relationship anatomy model and FIGS. 2.1.0.0 through 2.4.0.0 to classify multiple states of mind for every fundamental category and each of fundamental category's motivational coping techniques. In addition, using these classifications and the design of the relationship anatomy model, the Table B—Discussion Generator constructs the possible inquiries for each state of mind based on a plurality of variables including the attitudinal trait descriptions that can be generated back to the user during a users evaluation of a discussion topic.

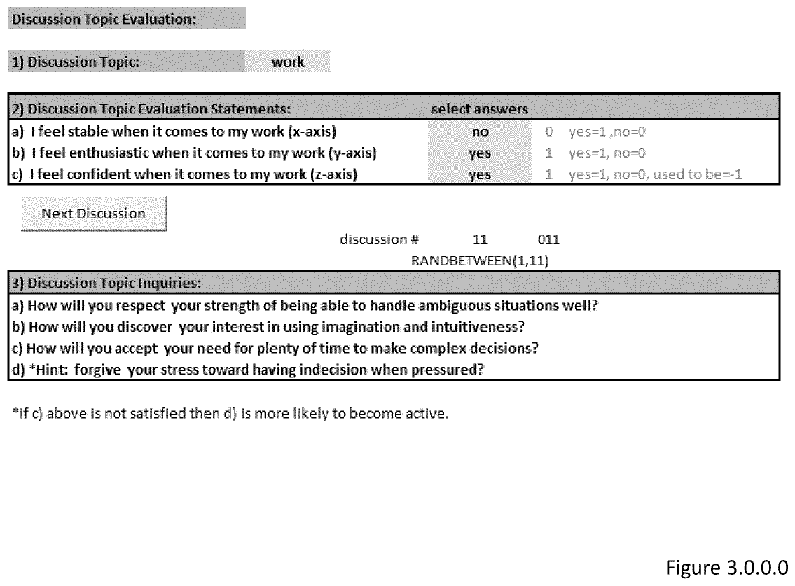

Table C—User Presentation—The Table shows the user interface of the system wherein the user defines a discussion topic which they wish to investigate, answers three discussion topic evaluation statements, and where a series of inquiries, one for each fundamental category, are generated from the Table B—Discussion Generator. As briefly described for FIG. 3.0.0.0 above, the user reads the inquiries and investigates answers to the inquiries that are intended to help them evoke ways to improve their situations or relationships regarding the discussion topic. In addition, the user may click a button called “next discussion” which changes the attitudinal trait descriptions built into the inquiries that are generated from the discussion generator. This enables a user to generate alternate inquiries to increase the possibility of discovering the most optimum way to improve their situations or relationships regarding the discussion topic.

### DETAILED DESCRIPTION OF THE INVENTION

There are two major sections described in the remainder of this summary section hereinbelow:
A. Section A describes the mechanics of the invention as it relates to the Relationship Anatomy Model in regards to the fundamental categories, motivational attributes, motivational coping techniques, and the user's motivation(s) or attitudinal trait descriptions (e.g., descriptions A & B) relative to a discussion topic.
B. Section B describes the mechanics of the invention as it relates to how a user interacts with this system in everyday situations, relationships, or aspects of general life (e.g., discussion topics) through the User Presentation.

### Section A—Mechanics of the Invention

Section A describes the mechanics of the invention as it relates to the design of the relationship anatomy model in regards fundamental categories, motivational attributes, motivational coping techniques and to the user's description of themselves in selecting predefined attitudinal trait descriptions. In order to appreciate these mechanics of the present invention, refer to the structural components of the relationship anatomy model embodiment shown in FIG. 2.0.0.0 through 2.4.0.0 and to Table A—Description Construct Table and Table B—Discussion Generator. The mechanics of this invention are described in 1 through 10 below:

1. Fundamental Category

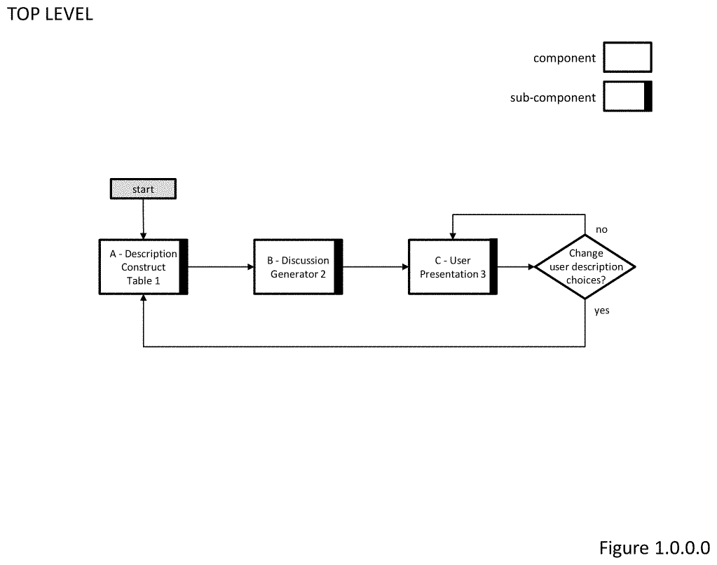

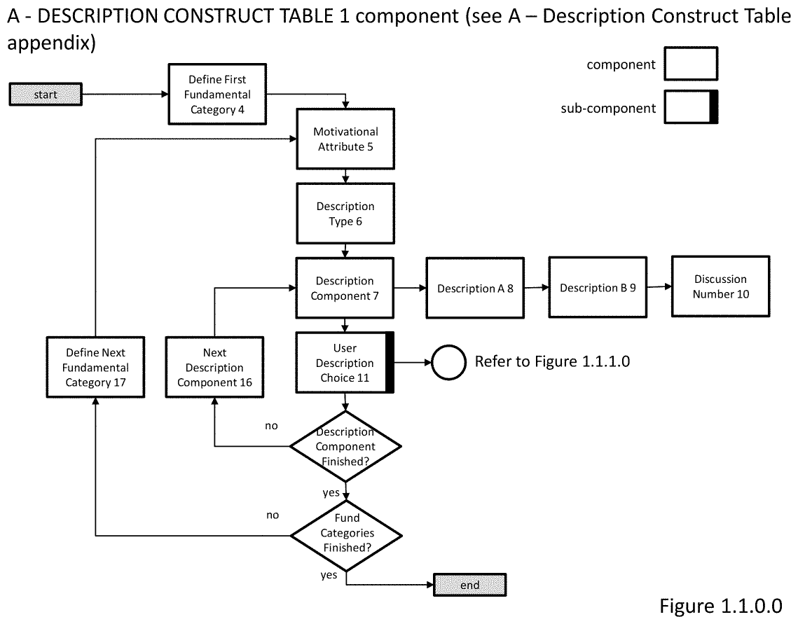

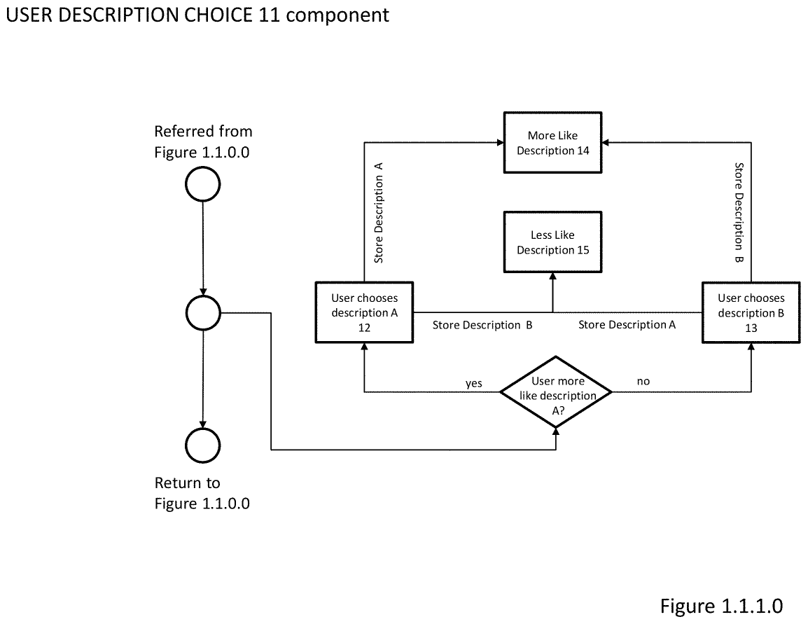

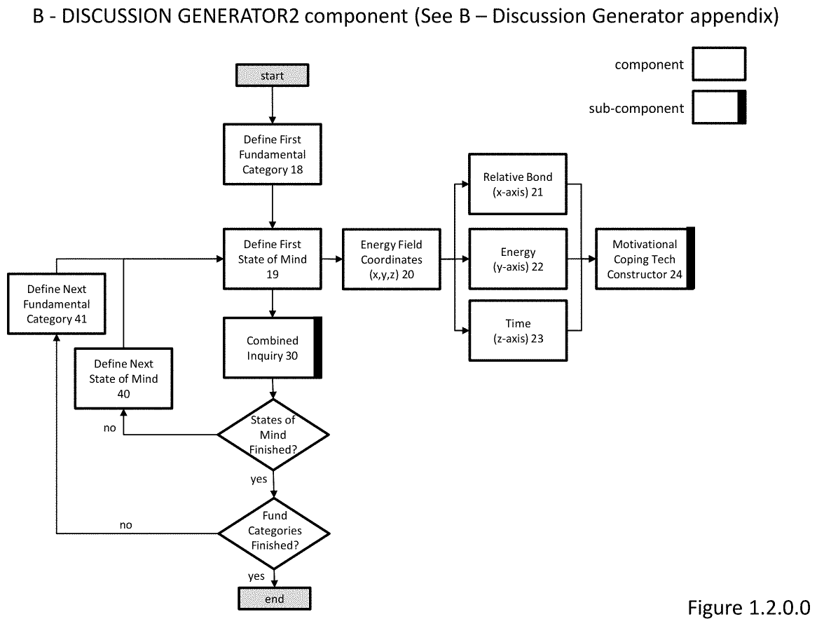

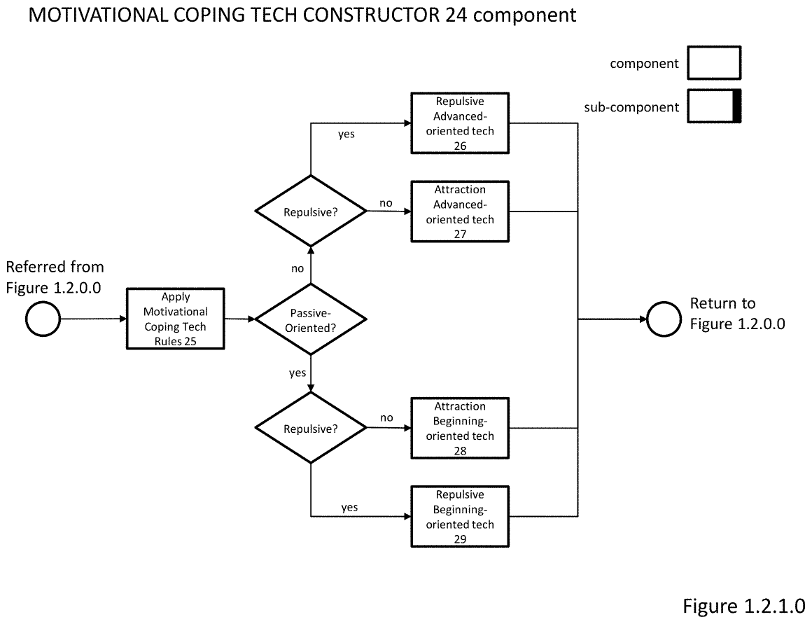

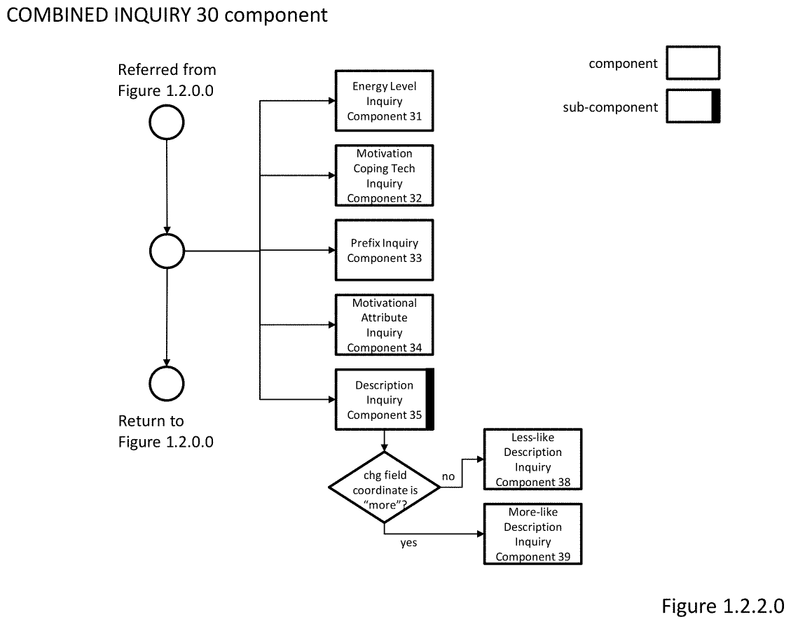

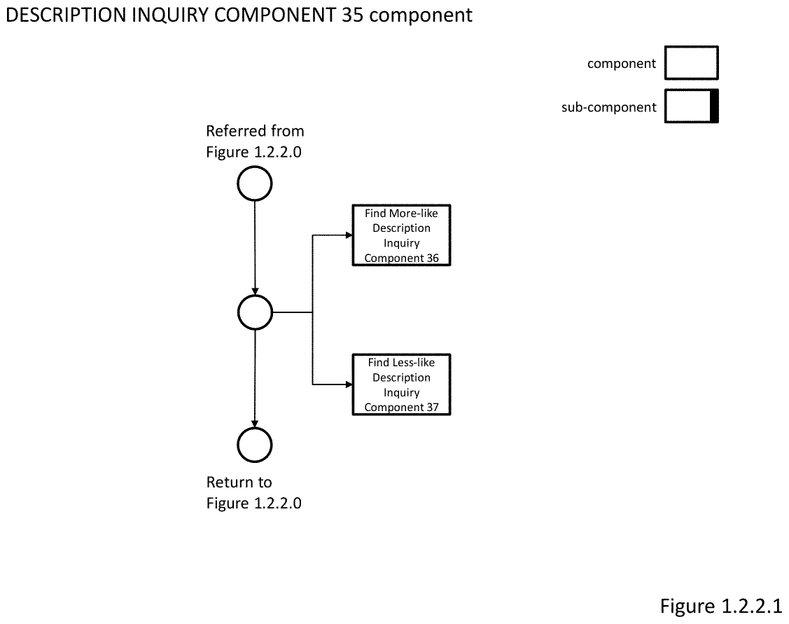

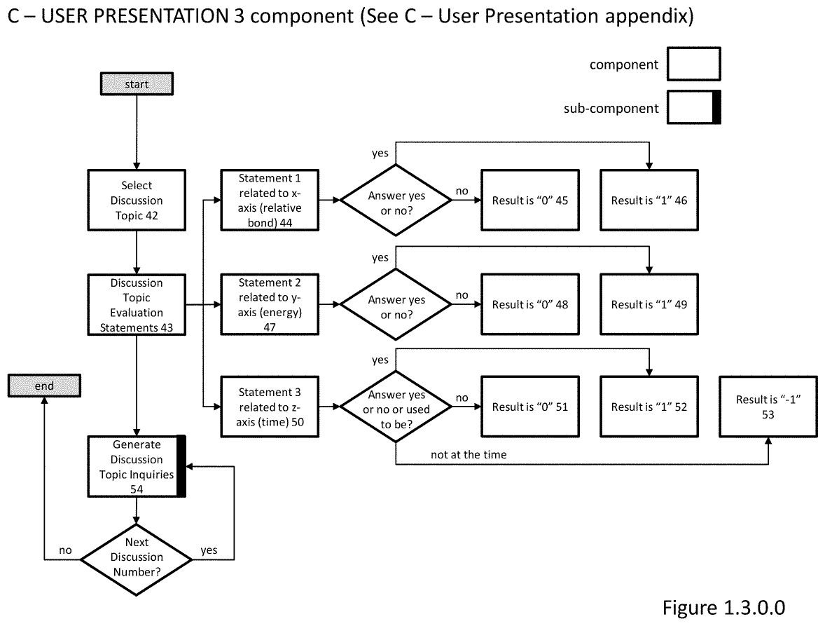

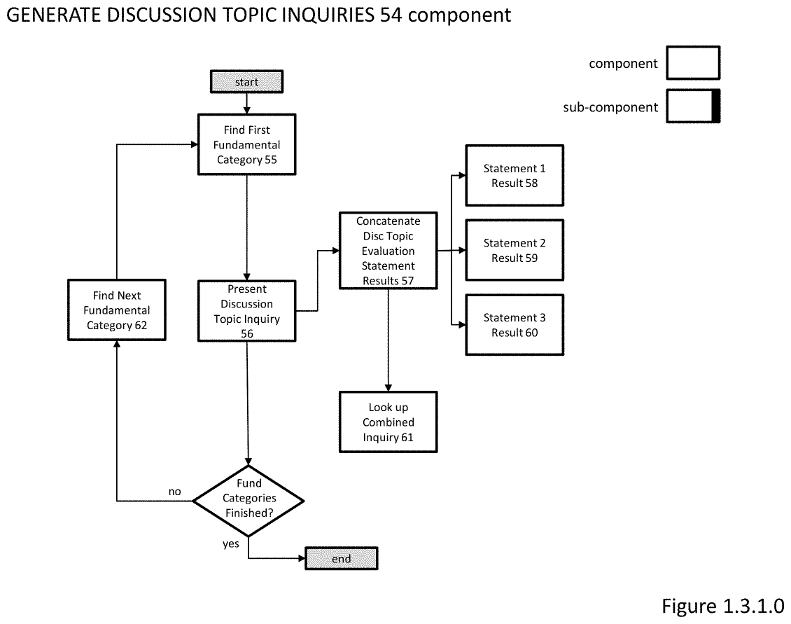

One aspect of the Relationship Anatomy Model presents visual instances of pre-defined relating qualities in the form of particular relating concepts (denoted as “fundamental categories” herein). The facilitator and the user of the system define such fundamental categories through the A—DESCRIPTION CONSTRUCT TABLE 1 component in FIG. 1.0.0.0. Also, once the fundamental categories are classified and defined, they are stored in a table called the Table A—Description Construct Table wherein the fundamental categories are established and entered in the Table A—Description Construct Table described in subsection 3 below and through the DESCRIPTION CONSTRUCT TABLE 1 component in FIG. 1.0.0.0. As an example of such a fundamental category, there may be a fundamental category indicative of the users “confidence”. In addition, such a fundamental category (e.g., confidence) has a one-to-one relationship with a motivational attribute (e.g., needs) as primarily defined through the MOTIVATION ATTRIBUTE 5 component in FIG. 1.1.0.0 as described in more detail in subsection 2 immediately below. That is, such a “confidence” fundamental category may be useful for assessing the degree to which a user tends (or defaults) to assess a preferred outcome (e.g., satisfying a “need” instance) to a wide range of discussion topics. Thus, such a “confidence” fundamental category may be useful for determining, given the uncertainty of future circumstances, the propensity of the user to generally ascribe a greater weight to a preferred outcome than to a non-preferred outcome based on the recognition or awareness of the user's “more like” or “less like” set of needs. Additionally, such a need motivational attribute (as described in more detail in subsection 2 immediately below) provides attitudinal trait descriptions through the DESCRIPTION COMPONENT 7 component in FIG. 1.1.0.0 along with a pre-defined motivational coping technique(s) defined through the MOTIVATIONAL COPING TECH CONSTRUCTOR 24 component in FIG. 1.2.0.0 to support a user's “confidence” development or evolution in aspects of a particular discussion topic (e.g., the user's tendency to ascribe a preferred outcome to a future situation or relationship) in relation to a user's “more like” or “less like” specific needs. Thus, the term “confidence” (e.g., a fundamental category) in the present discussion may have the following description:
1.1. Confidence: to have trust or faith in something or someone. More particularly, whether (or to what degree) the user's perception of the conditions, generated substantially by the discussion topic (or substantially derived therefrom), are or will ultimately be consistent with the user's expectations (e.g., needs) of the conditions.

Note that in one or more embodiments of the invention, additional fundamental categories may be identified by the words: patience, devotion, and honor, wherein such additional categories may be described as follows:
1.2. Patience: bearing or enduring emotional pain, difficulty, provocation, or annoyance with calmness. More particularly, whether (or to what degree) the user's perception of undesirable conditions created by the discussion topic (or substantially derived therefrom), are generally (e.g., substantially consistently) accepted without substantial increase in the user's “more like” or “less like” stress reactions (e.g., as described in subsection 2 immediately below, “stress” reactions are defined as a motivational attribute for the fundamental category Patience).
1.3. Devotion: ardent, often selfless affection and dedication, as to a person or principle; love. More particularly, whether (or to what degree) the user perceives that he/she is actively interested in the discussion topic (or conditions arising that are related thereto) so that the user explores and/or sets goals that are related to the discussion topic as it relates to the user's “more like” or “less like” interests (e.g., as described in subsection 2 immediately below, “interests” are defined as a motivational attribute for the fundamental category Devotion).
1.4. Honor high respect, as that shown for special merit; esteem. More particularly, whether (or to what degree) the user perceives that he/she is incorporating their usual strengths within the discussion topic (or conditions arising that are related thereto) as they relate to the user's “more like” or “less like” usual strengths (e.g., as described in subsection 2 immediately below, “strengths” are defined as a motivational attribute for the fundamental category Honor).

Of course, additional and/or alternative fundamental categories may be used with the present invention as one skilled in the art will understand once the scope of the invention is fully appreciated. For example, such additional and/or alternative fundamental categories may be categories that represent learning new skills such as learning to grow a garden. Such fundamental categories for learning to grow a garden may be defined by a facilitator and a user of the present invention as preparedness, accumulation, designation, and maintenance (e.g., there may be more or fewer fundamental categories define). Or another example may be learning the most efficient and effective means of managing to re-engineer an existing structure. Such fundamental categories that are representative of the most efficient and effective means to re-engineer an existing structure may be defined by a facilitator and a user of the present invention as salvage-ability, recyclable, fabrication, and environment. As one skilled in the art will understand, there are an unlimited number of applications that may be used for this invention.

2. Motivational Attribute

Another aspect of the Relationship Anatomy Model is that for each such fundamental category (e.g., “confidence”) described above and defined through the A—DESCRIPTION CONSTRUCT TABLE 1 component in FIG. 1.0.0.0, the present invention may associate one or more motivational attributes (e.g., “needs”) for each fundamental category. The motivational attributes described in 2.1 through 2.5 below are defined within the MOTIVATION ATTRIBUTE 5 component in FIG. 1.1.0.0, and are directly in relationship with the fundamental category. However, such motivational attributes may be modified by the facilitator and the user, and are associated with at least one (and preferably each of a plurality) fundamental category as defined by the facilitator through the A—DESCRIPTION CONSTRUCT TABLE 1 component in FIG. 1.0.0.0 and more specifically through the DEFINE FIRST FUNDAMENTAL CATEGORY 4 component and the DEFINE NEXT FUNDAMENTAL CATEGORY 17 component in FIG. 1.1.0.0. Also, the motivational attributes are classified and stored in a table called the Table A—Description Construct Table wherein the motivational attributes are defined and organized through the MOTIVATIONAL ATTRIBUTE 5 component in FIG. 1.1.0.0.

A “motivational attribute” facilitates and is essential for the development or evolution of its corresponding fundamental category within a user. In particular, such motivational attributes classify users “more like” or “less like” attitudinal trait descriptions as described in subsection 1 above in this section which provide additional guidance in establishing, in guiding the development or evolution of, and/or using motivational coping techniques that are intended to facilitate generation inquiries related to the fundamental category regarding the users experience of a discussion topic. Examples of such motivational attributes for fundamental categories as described above are as follows.
a. For the above mentioned fundamental category of “confidence”, the corresponding motivational attribute is denoted as “need”, wherein “need” may be described as follows (for a given discussion topic):
   The user's experience of what is considered as a necessary (or required) environment and/or necessary outcome for the discussion topic (or derived substantially therefrom) so that the user perceives, or may choose to experience a greater motivation, either: (a) to initiate or to not abandon the discussion topic, or (b) to terminate activities related to the discussion topic.
   Note, as an aside, the rationale for identifying “need” as a motivational attribute for the fundamental category of “confidence” is that it is believed that a users confidence, as related to a discussion topic, may be operably assessed by determining the degree to which the users or other's needs (related to the discussion topic) are perceived by the user as being accommodated or satisfied.
b. In one embodiment of the invention further described below, the above mentioned fundamental category of “patience” has an associated motivational attribute denoted “stress” reactions, wherein “stress” reactions may be described as follows (for a given discussion topic):
   The user's or others stress reactions related to the discussion topic (and/or outcomes therefrom) resulting from undesirable or unexpected conditions or circumstances created by the discussion topic (or substantially derived therefrom).
   Note, as an aside, the rationale for identifying “stress” reactions as a motivational attribute for the fundamental category of “patience” is that it is believed that a user's or others patience, as related to a discussion topic, may be assessed by determining the degree to which the users or other's needs, not stress reactions, related to the discussion topic are perceived by the user as being accommodated or satisfied. In other words, when a user's or others needs (as related to a discussion topic) are being accommodated or satisfied then it is believed that a user's or others stress reactions are minimized or at least reduced within the discussion topic.
c. In one embodiment of the invention further described below, the above mentioned fundamental category of “devotion” has an associated motivational attribute denoted “interests”, wherein “interests” may be described as follows (for a given discussion topic):
   The users or other's interests related to the discussion topic may be described as “active” or “inactive” within the discussion topic (and/or conditions arising that are related thereto). In particular, the present invention allows the user to explore ways to elicit, change or accept his/her or others interests (i.e., active or inactive) in the content of the discussion topic (or conditions arising that are related thereto).
   Note, as an aside, the rationale for identifying “interests” as a motivational attribute for the fundamental category of “devotion” is that it is believed a users or other's devotion, as related to the discussion topic, may be assessed by determining the degree to which the user utilizes their own or others interests as related to the discussion topic.
d. In one embodiment of the invention further described below, the above mentioned fundamental category of “honor” has an associated motivational attribute denoted “strengths”, wherein “strengths” may be described as follows (for a given discussion topic):
   The users or other's strengths related to the discussion topic (and/or outcomes therefrom) resulting in incorporating the users or other's strengths on conditions or circumstances created by the discussion topic (or substantially derived therefrom).
   Note, as an aside, the rationale for identifying “strengths” as a motivational attribute for the fundamental category of “honor” is that it is believed a user's honor, as related to the discussion topic, may be assessed by determining the degree to which the user utilizes their own or others strengths as related to the discussion topic. Also, it is believed that when a users or other's needs described in 2.1 above are being accommodated and satisfied it is more likely that a user's or others strengths are stimulated

3. Description Construct Table

The Fundamental Categories, Motivational Attributes, and Attitudinal Trait Descriptions described above in section A and throughout this invention are organized and populated in a structure called the Description Construct Table in Table A—Description Construct Table and is processed by the A—DESCRIPTION CONSTRUCT TABLE 1 component in FIG. 1.0.0.0 and by the USER DESCRIPTION CHOICE 11 component in FIG. 1.1.1.0.

In general the description construct table serves two purposes. The first purpose of the description construct table is that it organizes and stores pre-defined pairs of attitudinal trait descriptions (e.g., description A & B) for multiple classifications. These pre-defined pairs of attitudinal trait descriptions are classified by fundamental category, motivational attribute, description type, and description component (refer to each classification listed in the terms and definitions section for a more detailed description of each). For at least one embodiment of this invention, the attitudinal trait descriptions are pre-defined by the facilitator for every classification listed immediately above. A pair of attitudinal trait descriptions is described as having a binary or opposite effect. For example, within the fundamental category “confidence” where “need” is the motivational attribute, one pair of “need” attitudinal trait descriptions may be presented to a user as “reducing issues to their simplest form” (e.g., description A) and “plenty of time to make complex decisions” (e.g., description B). Meaning, either a user needs “reducing issues to their simplest form” or they need “plenty of time to make complex decisions”. This pair of attitudinal trait descriptions is designed to be binary or opposite in effect wherein a user likely possesses one over the other, but not needing both. For example, the need of “reducing issues to their simplest form” most likely applies to people who need to make quick decisions whereas the need of “plenty of time to make complex decisions” most likely applies to people who need time to make decisions. As a result, the first purpose of the description construct table is to present the user with pairs of attitudinal trait descriptions that are classified by fundamental category, motivational attribute, description type, and description component (refer to each classification listed in the terms and definitions section for a more detailed description of each). The second purpose of the description construct table is that it becomes the intake process for the user (e.g., user's intake process). In other words, prior to using the system, the user prepares the system with information needed to make the system work. This is called the intake process wherein the user chooses from a list of paired attitudinal trait descriptions. For each pair of attitudinal trait descriptions (e.g., description A & B), the user chooses one of the pair that is “more-like” themselves. The user reads description A and then description B. Next the user decides which description is “more-like” themselves and they enter either “A” or “B” into a field called description choice evaluation procedure wherein the user enters “A” if description A is “more-like” themselves or the user will enter “B” if description B is “more-like” themselves. By default, the description not chosen from the pair becomes classified as “less-like” themselves. As a result, the second purpose provides the user a means of describing themselves by choosing which attitudinal trait description of the pair is “more like” themselves (e.g., description A or B). Once chosen, the “more like” user attitudinal trait descriptions are categorized as more like descriptions and the “less like” user attitudinal trait descriptions are categorizes as less like descriptions. As shown in FIG. 2.0.0.0 under substructure 1, the relationship anatomy model defines the more like descriptions (i.e., “more like” the user) on the right side of the model and the relationship anatomy model defines the less like descriptions (i.e., “less like” the user) on the left side of the model.

Another aspect of the description construct table includes that for a group of attitudinal trait descriptions for a given fundamental category is either considered “independent” from (e.g., “honor” “patience”, and “confidence”) or “dependent” on (e.g., “patience”) another series of corresponding attitudinal trait descriptions from another fundamental category (e.g., “confidence”). The “independent” and “dependent” fundamental category attitudinal trait descriptions are identified in the Table A—Description Construct Table in the description type field. “Independent” type fundamental categories and their attitudinal trait descriptions work independently, meaning they don't depend on another fundamental category to become active and the “dependent” type fundamental categories and their attitudinal trait descriptions depend on another fundamental category(ies) attitudinal trait descriptions to become active. For example, for each description component (see Table A—Description Construct Table) the fundamental category “patience” including its “stress” attitudinal trait descriptors (e.g., impatient with group interaction) is “dependent” on becoming apparent from the fundamental category “confidence” and its “need” attitudinal trait descriptors (e.g., “plenty of time alone or in small groups”). In other words, for each description component if a “need” attitudinal trait description (e.g., “plenty of time alone or in small groups”) is not met from the fundamental category “confidence” then the “dependent” “stress” attitudinal trait description (e.g., impatient with group interaction) is expected to become apparent. Therefore, the “stress” response attitudinal trait description from the fundamental category “patience” depends on the “need” attitudinal trait descriptions being met. Moreover, for each description component (e.g., Acceptance), if the “need” attitudinal trait description (e.g., “plenty of time alone or in small groups”) is NOT met then the corresponding description component's (e.g., Acceptance) “stress” attitudinal trait description (e.g., impatient with group interaction) in the patience fundamental category will most likely become apparent. Last, in the case where a “dependent” fundamental category and its attitudinal trait descriptions exists, the description type in the Table A—Description Construct Table is labeled with a letter at the end of the description type (e.g., “dependentA”). In addition, the “independent” fundamental category and its attitudinal trait descriptions for which the “dependent” category is dependent upon in the Table A—Description Construct Table is labeled with a letter at the end of the description type (e.g., “independentA”). This label helps to identify which “independent” categories belong to the corresponding “dependent” categories. In cases where there are multiple “dependent” categories the description type for each “independent” and its corresponding “dependent” category is labeled with a B, C, D, etc. . . . As a result of having “dependent” attitudinal trait descriptions, it is not necessary for the user to choose the attitudinal trait descriptions of “dependent” attitudinal trait descriptions. Through the description component and description type classifications a user making the choices of descriptions “more-like” themselves from the “independent” attitudinal trait descriptions is automatically choosing the attitudinal trait descriptions of the “dependent” attitudinal trait descriptions. For instance, every user choice in the field user choice evaluation procedure for the fundamental category “confidence” is replicated for the fundamental category “patience”.

Note that attitudinal trait descriptions for each motivational attribute may be substantially derived from an evaluation procedure or intake process, e.g., however, it is worth noting that assistance with such evaluation procedures may also be substantially in written form or automated, e.g., via software, and may be provided via a network such as the Internet. Thus, for the motivational attribute “needs”, there may be a collection of attitudinal trait descriptions that are consistent (e.g., statistically correspond) with a users “user profile” that when these attitudinal traits are supported (e.g., provided, and/or satisfied), the user is generally deemed to have a healthy outlook toward a given discussion topic or life in general. Alternatively or optionally, when these attitudinal trait descriptions are not supported (e.g., not provided and/or not satisfied), the user is generally deemed to have an unhealthy mental state-of-mind or outlook of a given discussion topic or life in general. For the motivational attribute “needs” and its corresponding fundamental category “confidence”, such “need” attitudinal trait descriptions may be, e.g.: needing plenty of time to make decisions, needing a busy schedule, or needing a definite plan in place to accomplish a task. Conversely, the opposite behavior to these descriptions could represent other's behaviors within a particular discussion topic. In addition, if an idealized user is aware of such needs, then the user is more likely to discover ways to accommodate these needs within a discussion topic, for either their own needs or other's needs. Moreover, it is believed that the user's confidence will develop or evolve over time within a discussion topic in correspondence to increased awareness of such needs and discovering ways to accommodate these needs. Also, it is believed that the needs which are not like the user (i.e., needs “less like” the user) may also provide the user with valuable insight. In other words, when the user shows a “lack of motivation” to a particular need, it may be that the user has either a preference for, or dislike of others having a strong propensity for having such a need. Accordingly, by allowing the user to investigate his/her perceptions regarding such “lack of motivation”, the user may gain useful personal insights. In particular, by presenting to the user presentations (e.g., questions, statements, etc.) such that various needs or attitudinal trait descriptions (which are different from those of the user), such different attitudinal trait descriptions can be explored by the user. Thus, the present invention may be used to help the user discover an increased awareness of not only his/her own needs or attitudinal trait descriptions as they relate to the users discussion topic(s), but also very different needs or attitudinal trait descriptions of others. In addition, the present invention may also help the user to become aware of another's behaviors and/or attitudes (e.g., attitudinal trait descriptions) and attempt to give the user a perception of how another person may relate to their own environments within a given discussion topic. In at least one embodiment of the present invention and described in more detail in subsection seven later in this section, a set of attitudinal trait descriptions (see Table A—Description Construct Table) are predefined and classified for the user as Description A and Description B. Each of these sets of attitudinal trait descriptions has an A description and a B description wherein the A description is defined as an opposite behavior/attitude of the B description. Furthermore, a computational system, through the USER DESCRIPTION CHOICE 11 component in FIG. 1.1.1.0 (see Table A—Description Construct Table) presents a collection of attitudinal trait descriptions which are selected by the user wherein the user focuses on choosing either A description to be like themselves or B description to be like themselves. The description that is not chosen by the user becomes the description “less like” themselves. Therefore, for each description component for every fundamental category, the user will have selected either A description or B description to be “more like” themselves and the description not chosen by the user will default to being “less like” themselves. For the purposes of explaining the relationship anatomy model and how all of its characteristics work together as one unit, the positive charge represents attitudinal trait descriptions “more-like” the user and the negative charge represents attitudinal trait descriptions “less-like” the user. A more detailed description or understanding of how these descriptions relate to the users discussion topic and how they are used in the inquiries related back to the user during the user presentation (see Table C—User Presentation) will be described later in Section B and throughout the embodiment of the invention.

4. Charge Fields (Attitudinal Trait Descriptions)

One aspect of the relationship anatomy model in FIG. 2.0.0.0 through 2.4.0.0 is to gather a collection of descriptions of “attitudinal traits” (denoted as “attitudinal trait descriptions”, “description A”, or “description B” herein) that represent what are believed to be descriptive of at least one idealized user, wherein such attitudinal trait descriptions are indicative of the idealized user's usual, default and/or preferred attitudes (and/or resulting behaviors) related to the motivational attributes described briefly above and in more detail later in this section for the corresponding fundamental categories also described above and in more detail later in this section. FIG. 2.0.0.0 represents the structural components or substructures of the Relationship Anatomy Model. More specifically, substructure 1, “Charge Fields”, categorizes the attitudinal trait descriptions as a result of the user selecting either description A or description B “more like” themselves for each description component and every fundamental category. The attitudinal trait descriptions are stored in the description construct table described in subsection 3 above (see Table A—Description Construct Table). Once the description A or description B is selected by the user during the intake process, both descriptions will be categorized as either a “Less like Description” or a “More like Description” wherein all “Less like Descriptions” are indicative of the users “less like” descriptions and all the “More like Descriptions” are indicative of the users “more like” descriptions. In the Relationship Anatomy Model (refer to FIG. 2.0.0.0 substructure 1), the “Less like Descriptions” reside on the left side or field of the x-axis and the “More like Descriptions” reside on the right side or field of the x-axis. Furthermore, the intended design in the relationship anatomy model regarding the Charge Fields are fields that are bound together by a positive (e.g., “more-like” descriptors) and negative (e.g., “less-like” descriptors) charge (hence Charge Fields). These fields are designed to be similar to magnetic fields. It is also important to note that in the description construct table for each fundamental category there are eleven description components wherein each description component (e.g., Acceptance) is comprised of one description for description A and one description for description B. During the user's intake process, for each description component within every fundamental category, the user will choose which description (A or B) is “more-like” themselves. The other description will default to being categorized as “less-like” themselves. Even though this invention could be designed to have more or less attitudinal trait descriptions listed or more or less fundamental categories, the application described for this invention requires the user to choose from forty-four pairs of attitudinal trait descriptions (e.g., eleven description components times four fundamental categories). In addition, each description component within each fundamental category is numbered, 1 through 11. This number is located in the description construct table under the discussion number field. As it will be explained further in section B (the User Presentation; refer to FIG. 3.0.0.0) later in this application, for each fundamental category the discussion number is used to generate from the discussion generator only one description at a time wherein this description is constructed into the inquiry which is generated back to the user based on a user's evaluation of a discussion topic.

5. Energy Fields

It is another aspect of the Relationship Anatomy Model in FIGS. 2.0.0.0 through 2.4.0.0 that there are at least two types of “states of mind” for each fundamental category and its corresponding motivational attribute. In FIG. 2.0.0.0 energy fields are shown under Substructure 2. It is important to note that these energy fields operate independent from the Charge Fields described in subsection 1 of this section. As it will be described in more detail in subsection seven later in this section, these energy fields, in addition to other substructures in the Relationship Anatomy Model, aid the facilitator in defining the motivational coping techniques for each fundamental category. As described in the Terms and Definitions section under energyΔ (y), these two types of states of mind are “passive” (e.g., denoted by a lower Δ₁ energy) and “active” (e.g., denoted by a higher Δ₂ energy) states of mind. In addition, regarding the development of inquiries wherein the user reads these inquiries to investigate a particular discussion topic, a portion of the inquiry called the energy level inquiry component (see Table B—Discussion Generator), at least in one embodiment of the present invention, is developed based on the two energy levels wherein the lower Δ₁ energy is regarded as a more passive oriented inquiry that begins with “What” which assumes a user to be more inactive in their pursuit to discover answers in their investigation of the discussion topic. On the other hand, the higher Δ₂ energy is regarded as a more active oriented inquiry that begins with “How” which assumes a user to be more active in their pursuit to discover answers in their investigation of the discussion topic. Moreover, active and passive states of mind may be subdivided into the following regarding a user's discovery for answers regarding their investigation of a particular discussion topic. For the active state of mind, additional substates may be incorporated: active for avoidance, and active for pursuing. For the passive state of mind: passive from fear, passive from indifference, and passive from pleasure.

6. Technique Measures (x-axis & y-axis)

It is another aspect of the Relationship Anatomy Model to aid the facilitator in classifying and defining the motivational coping techniques described in subsection seven later in this section. The technique measures are presented in substructure 3 in FIG. 2.0.0.0 and are designed to not only aid in defining the motivational coping techniques described in subsection seven later in this section but are also designed to relate the users perception of a discussion topic through the discussion topic evaluation statements in the user presentation described in more detail through section B later in this section (refer to Table C—User Presentation). The design of the technique measures in the Relationship Anatomy Model in FIG. 2.0.0.0 through 2.4.0.0 shows at least four “quadrant caches” wherein these quadrant caches are divided up by two axes, the x-axis denoted herein as relative bond (x-axis) and the y-axis denoted herein as energy (y-axis). The relative bond x-axis is, in part, used to describe a classification of the facilitator and user defined motivational coping techniques which are defined in the Table B—Discussion Generator which are described in subsection seven later in this section. One side of the x-axis is valued as 0 and labeled as “attraction” wherein the “attraction” is designed to represent motivational coping techniques that exhibit a “spin up” directional (e.g., pulling) force. The other side of the x-axis is valued as 1 and labeled as “repulsive” wherein the “repulsive” label is designed to represent motivational coping techniques that exhibit a “spin down” directional (e.g., pushing) force. Together, a “spin up” directional force along with a “spin down” directional force represents a “bonding force” and momentum between two “linked” motivational coping techniques across the x-axis, both for lower energy level motivational coping techniques and for higher energy level motivational coping techniques wherein the lower energy motivational coping techniques are designed to simulate Cooper Pairs (a pair of electrons behavior in a superconductor) and the higher energy motivational coping techniques are designed to simulate quasi excitable pairs when the lower energy pair (i.e., Cooper pair) is struck by a phonon (the behavior of what happens to a cooper pair in a superconducting tunneling process). Another axis, the y-axis, represents energy (y) wherein the top of the y-axis is valued as 1, a higher energy, and the bottom of the y-axis is valued as 0, a lower energy. Not only do the motivational coping techniques have a particular relationship across the x-axis for every fundamental category (e.g., “bonding force”), but they also have a different type of relationship between the lower motivational coping techniques and their relative higher, or upper, motivational coping techniques across the y-axis for every fundamental category. For both sides of the x-axis, the lower energy motivational coping technique represents a “beginning level” motivational coping technique (e.g., “allow”) and the higher motivational coping technique, relative to its lower motivational coping technique, represents an “advanced-level” motivational coping technique (e.g., “accept”). A more detailed description of the motivational coping techniques design will become more apparent to the reader described later in subsection seven in this section.

7. Time Measures (z-axis)

It is another aspect of the Relationship Anatomy Model to classify time measures for relating inquiries back to the user wherein a present, past, or future timeframe is conveyed in the inquiry. The time measures are presented in substructure 4 in FIG. 2.0.0.0 through 2.4.0.0 and are designed to represents a plurality of time (z) measures for every fundamental category. In at least one embodiment of the present invention there are three time (z) measures, however, a plurality of time measures may be created. First, the front side of the z-axis represents “present time” with a measure value of 0. A second time measure on the back side of the z-axis represents “past time” with a measure value of −1. Third, the middle portion of the z-axis represents “future time” with a measure value of 1. For each time measure the relative bond (x-axis) and the energy (y-axis) is represented. The time (z-axis) measures are designed to relate the users perception of a discussion topic through the discussion topic evaluation statements in the user presentation. The design relates a timeframe component in each inquire generated back to the user wherein a timeframe is conveyed which is intended to place the users state of mind either in present time, past time, or future time. Time measures are described in more detail in section B later in this section.

So far in this section, 4 substructures of the relationship anatomy model have been described (see FIG. 2.0.0.0 through 2.4.0.0). At this point, in order to fully appreciate and effectively describe a plurality of variables and how they function, it is necessary to combine all 4 substructures described immediately above (see 1 through 4 above) which represent one structure denoted as the relationship anatomy model wherein, as shown in FIG. 2.0.0.0, the relationship anatomy model is composed of multiple characteristics with four quadrants. It is in these quadrants where the motivational coping techniques are defined. Defining motivational coping techniques will become more apparent later in subsection seven of this section.

8. Relationship Anatomy Model (Combining Subsections 4 Through 7 Above)

In summary, 4 substructures that represent the relationship anatomy model have been described in substructures 4 through 7 above. First, subsection 4 of section A above describes substructure 1 shown in FIG. 2.0.0.0 where charge fields are classified to include a plurality of mutually exclusive classifications for the attitudinal trait descriptions, both for “more like descriptions” and “less like descriptions” (i.e., set of attitudinal trait descriptions) wherein the more like descriptions represent attitudinal traits “more like” the user (represented on the right side of the x-axis) and the less like description represent attitudinal traits “less like” the user (represented on the left side of the x-axis). Second, subsection 5 of section A above describes substructure 2 shown in FIG. 2.0.0.0 where two energy fields represent at least two types of “states of mind”, or energyΔ (y). A “higher Δ₂ energy” state of mind, or active-oriented state of mind, is represented on the top or upper portion of the y-axis and a “lower Δ₁ energy” state of mind, or passive-oriented state of mind, is represented on the bottom or lower portion of the y-axis. Thirdly, subsection 6 in section A above describes substructure 3 shown in FIG. 2.0.0.0 wherein substructure 3 presents technique measures that classify motivational coping techniques in four quadrants or “quadrant caches”. Last, subsection 7 in section A above describes substructure 4 shown in FIG. 2.0.0.0 wherein substructure 4 presents time measures that classify timeframes for relating inquiries generated back to the user that are intended to convey present, past and future time.

Note that for describing the present invention, the arrangement or geometry of the relationship anatomy model disclosed in FIG. 2.0.0.0 through 2.4.0.0 and described in subsections 4 through 7 above is relied upon to simplify the description herein. However, such simplification should not be interpreted as a limitation of the invention. Indeed, there could be more or fewer than four quadrants in the Relationship Anatomy Model. For example, a user may only want to design one quadrant cache for every motivational attribute (described in subsection 2 above) and fundamental category (described in subsection 1 above) wherein the user desires to only focus on one classification of their attitudinal trait descriptions. In addition, a user may want to design a subset of quadrant caches for each of the four existing quadrant caches wherein each subset of quadrant caches classifies a “deeper” sense or description of the attitudinal trait description sets or a “deeper” sense of the motivational coping techniques (described in subsection 9 later in this section). In other words, there are an unlimited number of ways to define the “variables” within the structure of the relationship anatomy model wherein variables represent words, terms, and phrases that may change depending on the users preferences when defining such variables with the facilitator. The following description explains the classification of the relationship anatomy model design in FIG. 2.0.0.0 through 2.4.0.0. As stated above, these figures are provided to simplify the description herein. Also, it may be important to note that the relationship anatomy model design is incorporated within the Table A—Description Construct Table and Table B—Discussion Generator.

Quadrant Caches
FIG. 2.0.0.0 shows the basic structure of the four quadrant caches in the relationship anatomy model. FIG. 2.1.0.0 through 2.4.0.0 shows how the relationship anatomy model is structured for each fundamental category and its associated motivational attribute. A description of the four quadrant caches are described below and how they relate to the users evaluation of the discussion topic, the user selection of attitudinal trait descriptions, and the motivational coping techniques:
Quadrant 1 Cache: A quadrant whereby the user has a “low” energy in the context of a discussion topic, and the attitudinal trait descriptions represent instances that are relatively “more-like” the user (more like descriptions). Inquiries generated back to the user are intended to relate a “passive-oriented” state of mind. For present and past timeframes, motivational coping techniques are designed for each fundamental category (e.g., “confidence”) that intend to convey a “repulsive” or extrinsic beginning-level relating action (e.g., “support”). For future timeframes, motivational coping techniques are designed for each fundamental category (e.g., “confidence”) that intend to convey an “attraction” or intrinsic beginning-level related action (e.g., “allow”).
Quadrant 2 Cache: A quadrant whereby the user has a “low” energy in the context of a discussion topic, and the attitudinal trait descriptions represent instances that are relatively “less-like” the user (less like descriptions). Inquiries generated back to the user are intended to relate a “passive-oriented” state of mind. For present and past timeframes, motivational coping techniques are designed for each fundamental category (e.g., “confidence”) that intend to convey an “attraction” or intrinsic beginning-level relating action (e.g., “allow”). For future timeframes, motivational coping techniques are designed for each fundamental category (e.g., “confidence”) that intend to convey a “repulsive” or extrinsic beginning-level related action (e.g., “support”).
Quadrant 3 Cache: A quadrant whereby the user has a “high” energy in the context of a discussion topic, and the attitudinal trait descriptions represent instances that are relatively “more-like” the user (more like descriptions). Inquiries generated back to the user are intended to relate an “active-oriented” state of mind. For present and past timeframes, motivational coping techniques are designed for each fundamental category (e.g., “confidence”) that intend to convey a “repulsive” or extrinsic relating advanced-level action (e.g., “maintain”). For future timeframes, motivational coping techniques are designed for each fundamental category (e.g., “confidence”) that intend to convey an “attraction” or intrinsic advanced-level related action (e.g., “accept”).
Quadrant 4 Cache: A quadrant whereby the user has a “high” energy in the context of a discussion topic, and the attitudinal trait descriptions represent instances that are relatively “less-like” the user (less like descriptions). Inquiries generated back to the user are intended to relate an “active-oriented” state of mind. For present and past timeframes, motivational coping techniques are designed for each fundamental category (e.g., “confidence”) that intend to convey an “attraction” or intrinsic advanced-level relating action (e.g., “accept”). For future timeframes, motivational coping techniques are designed for each fundamental category (e.g., “confidence”) that intend to convey a “repulsive” or extrinsic advanced-level related action (e.g., “maintain”).

Wave Functions |ψ> (Concatenate Energy Field Coordinates)

Also, referring to FIG. 2.1.0.0 through 2.4.0.0 a different view of the Relationship Anatomy Model is illustrated that shows the classifications of quadrant caches in relationship to the energy field coordinates in Table B—Discussion Generator, the relative bond (x-axis), the energy (y-axis), and the time (z-axis). For every fundamental category each quadrant cache is classified with a triplet (e.g., 000 is an example of such a triplet), wherein the first coordinate of the triplet identifies the value related to the relative bond (x-axis) (e.g., 0 or 1), the second coordinate of the triplet identifies the value related to the energy (y-axis) (e.g., 0 or 1), and the third coordinate of the triplet identifies the value related to time (z-axis) (e.g., 0 or 1 or −1). Moreover, each quadrant cache (e.g., quadrant cache 1) is identified by a different triplet relative to every time (z-axis) measure, wherein the third coordinate of each triplet representing time (z-axis) identifies every quadrant cache in three or more different times. For at least one embodiment of the present invention, each quadrant cache is represented in present time (0), future time (1), and past time (−1). For each fundamental category there are 12 possible energy field coordinates that represent a triplet or wave function |ψ> position in the relationship anatomy model wherein each wave function |ψ> (e.g., for the 000 triplet the corresponding wave function is represented as |000>) is a possible outcome of the users perception related to a discussion topic. For the following, the process occurs in FIG. 1.2.0.0 ENERGY FIELD COORDINATES (X, Y, Z) 20 and 2.1.0.0 through 2.4.0.0 and Table B—Discussion Generator in the energy field coordinates field. For each fundamental category, every outcome or wave function |ψ> is identified below as (the following example below uses the motivational coping techniques from the fundamental category “confidence” in FIG. 2.3.0.0.):
   |100>: A quadrant cache 1 whereby the relative bond (x-axis) equals 1 (more like description), the energy (y-axis) equals 0 (lower energy; passive-oriented), and the time (z-axis) equals 0 (present time). This function represents motivational coping techniques that intend to convey a “repulsive” or extrinsic beginning-level related action (e.g., “support”).
   |000>: A quadrant cache 2 whereby the relative bond (x-axis) equals 0 (less like description), the energy (y-axis) equals 0 (lower energy; passive-oriented), and the time (z-axis) equals 0 (present time). This function represents motivational coping techniques that intend to convey an “attraction” or intrinsic beginning-level related action (e.g., “allow”).
   |110>: A quadrant cache 3 whereby the relative bond (x-axis) equals 1 (more like description), the energy (y-axis) equals 1 (higher energy; active-oriented), and the time (z-axis) equals 0 (present time). This function represents motivational coping techniques that intend to convey a “repulsive” or extrinsic advanced-level related action (e.g., “maintain”).
   |010>: A quadrant cache 4 whereby the relative bond (x-axis) equals 0 (less like description), the energy (y-axis) equals 1 (higher energy; active-oriented), and the time (z-axis) equals 0 (present time). This function represents motivational coping techniques that intend to convey an “attraction” or intrinsic advanced-level related action (e.g., “accept”).
   |001>: A quadrant cache 1 whereby the relative bond (x-axis) equals 0 (more like description), the energy (y-axis) equals 0 (lower energy; passive-oriented), and the time (z-axis) equals 1 (future time). This function represents motivational coping techniques that intend to convey an “attraction” or intrinsic beginning-level related action (e.g., “allow”). NOTE: you should notice that for future time motivational coping techniques (e.g., |001>, |101>, |011>, and |111>) the values as well as the motivational coping techniques on the relative bond x-axis change sides wherein the “attraction” motivational coping techniques with a value of 0 is on the right side representing “more-like” descriptions and the “repulsive” motivational coping techniques with a value of 1 is on the left side representing “less-like” descriptions. You should also notice that even though the values and the motivational coping techniques switch sides, the attitudinal trait descriptions don't change sides; they each represent a constant energy throughout each timeframe. This aspect of the relationship anatomy model is designed to represent momentum P and, depending on the users evaluation of the discussion topic, also enables any of the motivational coping techniques within a given fundamental category to represent either the “more-like” or the “less-like” attitudinal trait descriptions.
   |101>: A quadrant cache 2 whereby the relative bond (x-axis) equals 1 (less like description), the energy (y-axis) equals 0 (lower energy; passive-oriented), and the time (z-axis) equals 1 (future time). This function represents motivational coping techniques that intend to convey a “repulsive” or extrinsic beginning-level related action (e.g., “support”).
   |011>: A quadrant cache 3 whereby the relative bond (x-axis) equals 0 (more like description), the energy (y-axis) equals 1 (higher energy; active-oriented), and the time (z-axis) equals 1 (future time). This function represents motivational coping techniques that intend to convey an “attraction” or intrinsic advanced-level related action (e.g., “accept”).
   |111>: A quadrant cache 4 whereby the relative bond (x-axis) equals 1 (less like description), the energy (y-axis) equals 1 (higher energy; active-oriented), and the time (z-axis) equals 1 (future time). This function represents motivational coping techniques that intend to convey a “repulsive” or extrinsic advanced-level related action (e.g., “maintain”).
−|101>: A quadrant cache 1 whereby the relative bond (x-axis) equals 1 (more like description), the energy (y-axis) equals 0 (lower energy; passive-oriented), and the time (z-axis) equals −1 (past time). This function represents motivational coping techniques that intend to convey a “repulsive” or extrinsic beginning-level related action (e.g., “support”).
−|001>: A quadrant cache 2 whereby the relative bond (x-axis) equals 0 (less like description), the energy (y-axis) equals 0 (lower energy; passive-oriented), and the time (z-axis) equals −1 (past time). This function represents motivational coping techniques that intend to convey an “attraction” or intrinsic beginning-level related action (e.g., “allow”).
−|111>: A quadrant cache 3 whereby the relative bond (x-axis) equals 1 (more like description), the energy (y-axis) equals 1 (higher energy; passive-oriented), and the time (z-axis) equals −1 (past time). This function represents motivational coping techniques that intend to convey a “repulsive” or extrinsic advanced-level related action (e.g., “maintain”).
−|011>: A quadrant cache 4 whereby the relative bond (x-axis) equals 0 (less like description), the energy (y-axis) equals 1 (higher energy; active-oriented), and the time (z-axis) equals −1 (past time). This function represents motivational coping techniques that intend to convey an “attraction” or intrinsic advanced-level related action (e.g., “accept”).

9. Motivational Coping Techniques
First, it is important to note that even though motivational coping techniques are combined with attitudinal trait descriptions in an inquiry that is generated back to the user via a discussion topic evaluation the motivational coping techniques operate completely independent from the attitudinal trait descriptions. For the purpose of leading the reader into this section refer to FIG. 2.0.0.0 through 2.4.0.0, but specifically reference FIG. 2.0.0.0. It is also important that the reader understand the following wherein the relationship anatomy model is designed by words called motivational coping techniques that are bound together through meaningful relationships to one another. It was from the design of these words or motivational coping techniques which created the relationship anatomy model and all of its characteristics including the charge fields which creates the constant positive and negative charge energy in FIG. 2.0.0.0 labeled Substructure 1 (described in subsection 4 above), energy fields in FIG. 2.0.0.0 labeled Substructure 2 (described in subsection 5 above), technique measures in FIG. 2.0.0.0 labeled Substructure 3(described in subsection 6 above), and finally the time measures in FIG. 2.0.0.0 labeled Substructure 4 (described in subsection 7 above). Once these substructures are combined together it creates the relationship anatomy model module shown in FIG. 2.0.0.0 labeled relationship anatomy model which includes four quadrant caches (described in subsection 8a above). Each quadrant cache represents a motivational coping technique wherein each motivational coping technique and its meaning are defined primarily by Substructure 2—Energy Fields and Substructure 3—Technique Measures. These substructures provide the criteria which define each motivational coping technique for every fundamental category and its corresponding motivational attribute. The following describes the criteria from the relationship anatomy model that aid the facilitator in defining each the motivational coping techniques for its corresponding fundamental category.

a) Rule One
A motivational coping technique must be partially descriptive of the overall meaning associated with the fundamental category (e.g., “confidence”) and its corresponding motivational attribute (e.g., “need”).

b) Rule Two
A motivational coping technique must be descriptive of the energy fields as described in subsection 5 above. It is assumed that the “passive-oriented” or lower A₁ energy (y-axis) state of mind motivational coping techniques will be designed to be more inactive intended to convey reflection or thought as opposed to encouraging an action. In conjunction, “passive-oriented” motivational coping techniques should be more “beginning-oriented” wherein it conveys an easier way toward achieving the fundamental category (e.g., “confidence). Conversely, the “active-oriented” or higher Δ₂ energy (y-axis) state of mind motivational coping techniques will be designed to be more active intended to convey an action or activity. In conjunction, “active-oriented” motivational coping techniques should be more “advanced-oriented” wherein it conveys a way to finalize or complete achieving the fundamental category (e.g., “confidence”).

c) Rule Three
A motivational coping technique must be descriptive of the technical measures, more specifically, regarding the relative bond x-axis shown in Substructure 3 in FIG. 2.0.0.0 and described in subsection 6 above. It is assumed that the motivational coping techniques defined on the “attraction” side of the x-axis (e.g., “allow” and “accept”) convey an intrinsic action response. This type of action intends to encourage a user to absorb information (e.g., “pull-energy”; hence “attraction”); intending to take action based on information received from the environment. Conversely, it is assumed that the motivational coping techniques defined on the “repulsive” side of the x-axis (e.g., “support” and “maintain”) convey an extrinsic action response. This type of action intends to encourage a user to initiate an action or make an effort (e.g., “push-energy”; hence “repulsive”); intending to take an action or effort initiated by the user toward the environment.

d) Rule Four
As discussed in subsection 3 & 4 above in this section (it is encouraged to review these subsections before proceeding) wherein “Independent” type fundamental categories and their attitudinal trait descriptions work independently, meaning they don't depend on another fundamental category to become active and the “dependent” type fundamental categories and their attitudinal trait descriptions depend on another fundamental category(ies) attitudinal trait descriptions to become active. Rule four prescribes that, in addition, a series of motivational coping techniques for a given fundamental category is also, respectively, either considered “independent” from or “dependent” on another series of corresponding motivational coping techniques for another fundamental category. The independent categories are from the fundamental categories “honor”, “devotion”, and “confidence”. The dependent category in this invention is from the fundamental category “patience”. The independent fundamental category which “patience” is depended upon is “confidence”. Therefore, the motivational coping techniques that represent “patience” depend upon the motivational coping techniques that represent “confidence”. For example, first notice the “independent” motivational coping technique “accept” in Quadrant 4 Cache with a wave function of |010> from FIG. 2.3.0.0 (e.g., “accept” represents “need” attitudinal trait descriptions from the fundamental category “confidence”). Next, notice the corresponding “dependent” motivational coping technique “forgive” in Quadrant 4 Cache with the SAME wave function |010> from FIG. 2.4.0.0 (e.g., “forgive” represents “stress” reaction attitudinal trait descriptions from the fundamental category “patience”). In other words, the dependent motivational coping technique (e.g., “forgive”) and its “stress” attitudinal trait description(s) will only become active, or expressed, if the corresponding “independent” motivational coping technique (e.g., “accept”) “need” attitudinal trait description(s) are NOT met or satisfied. Therefore, Rule four prescribes that a series of motivational coping techniques for a given fundamental category is either considered “independent” from or “dependent” on another series of corresponding motivational coping techniques for another fundamental category.
As a result, when properly following the Rules One through Four above in the context of defining a series of motivational coping techniques for a given fundamental category in the Relationship Anatomy Model the users discussion topic evaluation (described in Section B below) will evoke learning and intended to evolve a complete understanding of a given fundamental category. In order to fully achieve an understanding of how to define the motivational coping techniques for each fundamental category, it is only necessary to explain the motivational coping techniques within the “present time” timeframe (e.g., z-axis) portion of the relationship anatomy is described for each fundamental category (e.g., “honor”, “devotion”, “confidence”, and “patience”; respectively refer to FIG. 2.1.0.0. through 2.4.0.0.):

Honor (Refer to FIG. 2.1.0.0) (High Respect, as that Shown Through Special Merit; Esteem)
Observe (to become aware of especially through careful and directed attention; notice) is the first motivational coping technique defined for the fundamental category “honor” and its associated motivational attribute “strength”. Regarding Rule One, to “observe” is intended to be partially descriptive of the fundamental category “honor” and its associated motivational attribute “strength” wherein the attitudinal trait descriptions defined for this fundamental category are considered strength descriptors. In other words, to “observe” something or somebody's “strengths” in situations or relationships is critical to developing ones strength attitudinal trait descriptors by increasing a users awareness of where strengths may or may not benefit a particular discussion topic related to this motivational coping technique. Regarding Rule Two, it is assumed that the motivational coping technique “observe” achieves a “passive-oriented” or lower A₁ energy (y-axis) state of mind wherein “to observe” achieves more inactivity intended to convey reflection or thought as opposed to encouraging an action. In addition, “to observe” achieves a more “beginning-oriented” motivational coping technique conveying an easier way to achieve learning or evolving the fundamental category within a particular discussion topic. Regarding Rule Three, it is intended that the motivational coping technique “observe” is a “repulsive” technique wherein it is assumed to convey an extrinsic action response. This type of action intends to encourage a user to initiate an action or make an effort (e.g., “push-energy”; hence “repulsive”); intending to take an action or effort initiated by the user toward the environment and/or others. Regarding Rule Four, it is intended that the motivational coping technique “observe” is an “independent” motivational coping technique wherein for its associated attitudinal trait descriptions to become apparent it does not require a different fundamental categories series of motivational coping techniques and its associated attitudinal trait descriptions to become apparent.
Appreciate (to recognize the quality, significance, or magnitude of; to be thankful or show gratitude for) is the second motivational coping technique defined for the fundamental category “honor” and its associated motivational attribute “strength”. Regarding Rule One, to “appreciate” is intended to be partially descriptive of the fundamental category “honor” and its associated motivational attribute “strength” wherein the attitudinal trait descriptions defined for this fundamental category are considered strength descriptors. In other words, to “appreciate” something or somebody's “strengths” in situations or relationships is critical to developing ones strength attitudinal trait descriptors by increasing a users awareness of where strengths may or may not benefit a particular discussion topic related to this motivational coping technique. Regarding Rule Two, it is assumed that the motivational coping technique “appreciate” achieves a “passive-oriented” or lower Δ₁ energy (y-axis) state of mind wherein “to appreciate” achieves more inactivity intended to convey reflection or thought as opposed to encouraging an action. In addition, “to appreciate” achieves a more “beginning-oriented” motivational coping technique conveying an easier way to achieve learning or evolving the fundamental category within a particular discussion topic. Regarding Rule Three, it is intended that the motivational coping technique “appreciate” is an “attraction” technique wherein it is assumed to convey an intrinsic action response. This type of action intends to encourage a user to absorb information (e.g., “pull-energy”; hence, “attraction”); intending to take an intrinsic action based on information received from the environment and/or others. Regarding Rule Four, it is intended that the motivational coping technique “appreciate” is an “independent” motivational coping technique wherein for its associated attitudinal trait descriptions to become apparent it does not require a different fundamental categories series of motivational coping techniques and its associated attitudinal trait descriptions to become apparent.
Admire (to regard with pleasure, wonder, and approval) is the third motivational coping technique defined for the fundamental category “honor” and its associated motivational attribute “strength”. Regarding Rule One, to “admire” is intended to be partially descriptive of the fundamental category “honor” and its associated motivational attribute “strength” wherein the attitudinal trait descriptions defined for this fundamental category are considered strength descriptors. In other words, to “admire” something or somebody's “strengths” in situations or relationships is critical to developing ones strength attitudinal trait descriptors by increasing a users awareness of where strengths may or may not benefit a particular discussion topic related to this motivational coping technique. Regarding Rule Two, it is assumed that the motivational coping technique “admire” achieves an “active-oriented” or higher Δ₂ energy (y-axis) state of mind wherein “to admire” is designed to be more active intended to convey an action or activity. In addition, “active-oriented” motivational coping techniques should be more “advanced-oriented” as compared to the “passive-oriented” techniques wherein if or when achieved it conveys a way to finalize or complete a higher awareness of the fundamental category. Regarding Rule Three, it is intended that the motivational coping technique “admire” is a “repulsive” technique wherein it is assumed to convey an extrinsic action response. This type of action intends to encourage a user to initiate an action or make an effort (e.g., “push-energy”; hence “repulsive”); intending to take an action or effort initiated by the user toward the environment and/or others. Regarding Rule Four, it is intended that the motivational coping technique “admire” is an “independent” motivational coping technique wherein for its associated attitudinal trait descriptions to become apparent it does not require a different fundamental categories series of motivational coping techniques and its associated attitudinal trait descriptions to become apparent.
Respect (to feel or show deferential regard for; esteem; to avoid violation of or interference with) is the fourth and last motivational coping technique defined for the fundamental category “honor” and its associated motivational attribute “strength”. Regarding Rule One, to “respect” is intended to be partially descriptive of the fundamental category “honor” and its associated motivational attribute “strength” wherein the attitudinal trait descriptions defined for this fundamental category are considered strength descriptors. In other words, to “respect” something or somebody's “strengths” in situations or relationships is critical to developing ones strength attitudinal trait descriptors by increasing a users awareness of where strengths may or may not benefit a particular discussion topic related to this motivational coping technique. Regarding Rule Two, it is assumed that the motivational coping technique “respect” achieves an “active-oriented” or higher A2 energy (y-axis) state of mind wherein “to respect” is designed to be more active intended to convey an action or activity. In addition, “active-oriented” motivational coping techniques should be more “advanced-oriented” as compared to the “passive-oriented” techniques wherein if or when achieved it conveys a way to finalize or complete a higher awareness of the fundamental category. Regarding Rule Three, it is intended that the motivational coping technique “respect” is an “attraction” technique wherein it is assumed to convey an intrinsic action response. This type of action intends to encourage a user to absorb information (e.g., “pull-energy”; hence, “attraction”); intending to take an intrinsic action based on information received from the environment and/or others. Regarding Rule Four, it is intended that the motivational coping technique “respect” is an “independent” motivational coping technique wherein for its associated attitudinal trait descriptions to become apparent it does not require a different fundamental categories series of motivational coping techniques and its associated attitudinal trait descriptions to become apparent.

Devotion (Refer to FIG. 2.2.0.0) (Ardent, Often Selfless Affection and Dedication, as to a Person or Principle; Synonym is Love)
a. Acknowledge (to admit the existence, reality, or truth of; to recognize as being valid or having force or power) is the first motivational coping technique defined for the fundamental category “devotion” and its associated motivational attribute “interest”. Regarding Rule One, to “acknowledge” is intended to be partially descriptive of the fundamental category “devotion” and its associated motivational attribute “interest” wherein the attitudinal trait descriptions defined for this fundamental category are considered interest descriptors. In other words, to “acknowledge” something or somebody's “interests” in situations or relationships is critical to developing ones interest attitudinal trait descriptors by increasing a user's awareness of where interests may or may not benefit a particular discussion topic related to this motivational coping technique. Regarding Rule Two, it is assumed that the motivational coping technique “acknowledge” achieves a “passive-oriented” or lower Δ₁ energy (y-axis) state of mind wherein “to acknowledge” achieves more inactivity intended to convey reflection or thought as opposed to encouraging an action. In addition, “to acknowledge” achieves a more “beginning-oriented” motivational coping technique conveying an easier way to achieve learning or evolving the fundamental category within a particular discussion topic. Regarding Rule Three, it is intended that the motivational coping technique “acknowledge” is a “repulsive” technique wherein it is assumed to convey an extrinsic action response. This type of action intends to encourage a user to initiate an action or make an effort (e.g., “push-energy”; hence “repulsive”); intending to take an action or effort initiated by the user toward the environment and/or others. Regarding Rule Four, it is intended that the motivational coping technique “acknowledge” is an “independent” motivational coping technique wherein for its associated attitudinal trait descriptions to become apparent it does not require a different fundamental categories series of motivational coping techniques and its associated attitudinal trait descriptions to become apparent.
Consider (to think carefully about) is the second motivational coping technique defined for the fundamental category “devotion” and its associated motivational attribute “interest”. Regarding Rule One, to “consider” is intended to be partially descriptive of the fundamental category “devotion” and its associated motivational attribute “interest” wherein the attitudinal trait descriptions defined for this fundamental category are considered strength descriptors. In other words, to “consider” something or somebody's “interests” in situations or relationships is critical to developing ones interest attitudinal trait descriptors by increasing a user's awareness of where interests may or may not benefit a particular discussion topic related to this motivational coping technique. Regarding Rule Two, it is assumed that the motivational coping technique “consider” achieves a “passive-oriented” or lower Δ₁ energy (y-axis) state of mind wherein “to consider” achieves more inactivity intended to convey reflection or thought as opposed to encouraging an action. In addition, “to consider” achieves a more “beginning-oriented” motivational coping technique conveying an easier way to achieve learning or evolving the fundamental category within a particular discussion topic. Regarding Rule Three, it is intended that the motivational coping technique “consider” is an “attraction” technique wherein it is assumed to convey an intrinsic action response. This type of action intends to encourage a user to absorb information (e.g., “pull-energy”; hence, “attraction”); intending to take an intrinsic action based on information received from the environment and/or others. Regarding Rule Four, it is intended that the motivational coping technique “consider” is an “independent” motivational coping technique wherein for its associated attitudinal trait descriptions to become apparent it does not require a different fundamental categories series of motivational coping techniques and its associated attitudinal trait descriptions to become apparent.
Fulfill (to bring into actuality; effect) is the third motivational coping technique defined for the fundamental category “devotion” and its associated motivational attribute “interest”. Regarding Rule One, to “fulfill” is intended to be partially descriptive of the fundamental category “devotion” and its associated motivational attribute “interest” wherein the attitudinal trait descriptions defined for this fundamental category are considered interest descriptors. In other words, to “fulfill” something or somebody's “interests” in situations or relationships is critical to developing ones interest attitudinal trait descriptors by increasing a user's awareness of where interests may or may not benefit a particular discussion topic related to this motivational coping technique. Regarding Rule Two, it is assumed that the motivational coping technique “fulfill” achieves an “active-oriented” or higher Δ₂ energy (y-axis) state of mind wherein “to fulfill” is designed to be more active intended to convey an action or activity. In addition, “active-oriented” motivational coping techniques should be more “advanced-oriented” as compared to the “passive-oriented” techniques wherein if or when achieved it conveys a way to finalize or complete a higher awareness of the fundamental category. Regarding Rule Three, it is intended that the motivational coping technique “fulfill” is a “repulsive” technique wherein it is assumed to convey an extrinsic action response. This type of action intends to encourage a user to initiate an action or make an effort (e.g., “push-energy”; hence “repulsive”); intending to take an action or effort initiated by the user toward the environment and/or others. Regarding Rule Four, it is intended that the motivational coping technique “fulfill” is an “independent” motivational coping technique wherein for its associated attitudinal trait descriptions to become apparent it does not require a different fundamental categories series of motivational coping techniques and its associated attitudinal trait descriptions to become apparent.
Discover (to notice or learn; especially by making an effort) is the fourth and last motivational coping technique defined for the fundamental category “devotion” and its associated motivational attribute “interest”. Regarding Rule One, to “discover” is intended to be partially descriptive of the fundamental category “devotion” and its associated motivational attribute “interest” wherein the attitudinal trait descriptions defined for this fundamental category are considered interest descriptors. In other words, to “discover” “something or somebody's “interests” in situations or relationships is critical to developing ones interest attitudinal trait descriptors by increasing a user's awareness of where interests may or may not benefit a particular discussion topic related to this motivational coping technique. Regarding Rule Two, it is assumed that the motivational coping technique “discover” achieves an “active-oriented” or higher Δ₂ energy (y-axis) state of mind wherein “to discover” is designed to be more active intended to convey an action or activity. In addition, “active-oriented” motivational coping techniques should be more “advanced-oriented” as compared to the “passive-oriented” techniques wherein if or when achieved it conveys a way to finalize or complete a higher awareness of the fundamental category. Regarding Rule Three, it is intended that the motivational coping technique “discover” is an “attraction” technique wherein it is assumed to convey an intrinsic action response. This type of action intends to encourage a user to absorb information (e.g., “pull-energy”; hence, “attraction”); intending to take an intrinsic action based on information received from the environment and/or others. Regarding Rule Four, it is intended that the motivational coping technique “discover” is an “independent” motivational coping technique wherein for its associated attitudinal trait descriptions to become apparent it does not require a different fundamental categories series of motivational coping techniques and its associated attitudinal trait descriptions to become apparent.

Confidence (Refer to FIG. 2.3.0.0) (Trust or Faith in a Person or Thing)
a. Support (to provide for) is the first motivational coping technique defined for the fundamental category “confidence” and its associated motivational attribute “need”. Regarding Rule One, to “support” is intended to be partially descriptive of the fundamental category “confidence” and its associated motivational attribute “need” wherein the attitudinal trait descriptions defined for this fundamental category are considered need descriptors. In other words, to “support” something or somebody's “needs” in situations or relationships is critical to developing ones need attitudinal trait descriptors by increasing a user's awareness of where needs may or may not benefit a particular discussion topic related to this motivational coping technique. Regarding Rule Two, it is assumed that the motivational coping technique “support” achieves a “passive-oriented” or lower Δ₁ energy (y-axis) state of mind wherein “to support” achieves more inactivity intended to convey reflection or thought as opposed to encouraging an action. In addition, “to support” achieves a more “beginning-oriented” motivational coping technique conveying an easier way to achieve learning or evolving the fundamental category within a particular discussion topic. Regarding Rule Three, it is intended that the motivational coping technique “support” is a “repulsive” technique wherein it is assumed to convey an extrinsic action response. This type of action intends to encourage a user to initiate an action or make an effort (e.g., “push-energy”; hence “repulsive”); intending to take an action or effort initiated by the user toward the environment and/or others. Regarding Rule Four, it is intended that the motivational coping technique “support” is an “independent” motivational coping technique wherein for its associated attitudinal trait descriptions to become apparent it does not require a different fundamental categories series of motivational coping techniques and its associated attitudinal trait descriptions to become apparent. However, this series of motivational coping techniques for the fundamental category “confidence” does have a “dependent” series of motivational coping techniques associated to it in the fundamental category “patience” described in iv) below. In other words, in order for the “stress” reaction attitudinal trait descriptions in the motivational coping techniques for the fundamental category “patience” described in iv) below to become apparent to the user during a discussion topic evaluation then it is most likely that the “need” attitudinal trait descriptions corresponding motivational coping techniques in the fundamental category “confidence” is NOT being satisfied or met. Therefore, it is necessary for the user to meet or satisfy the “need” attitudinal trait descriptions using the motivational coping technique “support” in the fundamental category “confidence” in order for the “stress” attitudinal trait descriptions from the corresponding “dependent” motivational coping technique in the fundamental category “patience” to not become apparent to the user during the discussion topic evaluation. As a result of meeting or satisfying the “need”, the “stress” is at least reduced or if not eliminated.
Allow (to let do or happen; permit) is the second motivational coping technique defined for the fundamental category “confidence” and its associated motivational attribute “need”. Regarding Rule One, to “allow” is intended to be partially descriptive of the fundamental category “confidence” and its associated motivational attribute “need” wherein the attitudinal trait descriptions defined for this fundamental category are considered strength descriptors. In other words, to “allow” something or somebody's “needs” in situations or relationships is critical to developing ones need attitudinal trait descriptors by increasing a user's awareness of where needs may or may not benefit a particular discussion topic related to this motivational coping technique. Regarding Rule Two, it is assumed that the motivational coping technique “allow” achieves a “passive-oriented” or lower Δ₁ energy (y-axis) state of mind wherein “to allow” achieves more inactivity intended to convey reflection or thought as opposed to encouraging an action. In addition, “to allow” achieves a more “beginning-oriented” motivational coping technique conveying an easier way to achieve learning or evolving the fundamental category within a particular discussion topic. Regarding Rule Three, it is intended that the motivational coping technique “allow” is an “attraction” technique wherein it is assumed to convey an intrinsic action response. This type of action intends to encourage a user to absorb information (e.g., “pull-energy”; hence, “attraction”); intending to take an intrinsic action based on information received from the environment and/or others. Regarding Rule Four, it is intended that the motivational coping technique “allow” is an “independent” motivational coping technique wherein for its associated attitudinal trait descriptions to become apparent it does not require a different fundamental categories series of motivational coping techniques and its associated attitudinal trait descriptions to become apparent. However, this series of motivational coping techniques for the fundamental category “confidence” does have a “dependent” series of motivational coping techniques associated to it in the fundamental category “patience” described in iv) below. In other words, in order for the “stress” reaction attitudinal trait descriptions in the motivational coping techniques for the fundamental category “patience” described in iv) below to become apparent to the user during a discussion topic evaluation then it is most likely that the “need” attitudinal trait descriptions corresponding motivational coping techniques in the fundamental category “confidence” is NOT being satisfied or met. Therefore, it is necessary for the user to meet or satisfy the “need” attitudinal trait descriptions using the motivational coping technique “allow” in the fundamental category “confidence” in order for the “stress” attitudinal trait descriptions from the corresponding “dependent” motivational coping technique in the fundamental category “patience” to not become apparent to the user during the discussion topic evaluation. As a result of meeting or satisfying the “need”, the “stress” is at least reduced or if not eliminated.
Maintain (to keep up or carry on; continue) is the third motivational coping technique defined for the fundamental category “confidence” and its associated motivational attribute “need”. Regarding Rule One, to “maintain” is intended to be partially descriptive of the fundamental category “confidence” and its associated motivational attribute “need” wherein the attitudinal trait descriptions defined for this fundamental category are considered need descriptors. In other words, to “maintain” something or somebody's “needs” in situations or relationships is critical to developing ones need attitudinal trait descriptors by increasing a user's awareness of where needs may or may not benefit a particular discussion topic related to this motivational coping technique. Regarding Rule Two, it is assumed that the motivational coping technique “maintain” achieves an “active-oriented” or higher Δ₂ energy (y-axis) state of mind wherein “to maintain” is designed to be more active intended to convey an action or activity. In addition, “active-oriented” motivational coping techniques should be more “advanced-oriented” as compared to the “passive-oriented” techniques wherein if or when achieved it conveys a way to finalize or complete a higher awareness of the fundamental category. Regarding Rule Three, it is intended that the motivational coping technique “maintain” is a “repulsive” technique wherein it is assumed to convey an extrinsic action response. This type of action intends to encourage a user to initiate an action or make an effort (e.g., “push-energy”; hence “repulsive”); intending to take an action or effort initiated by the user toward the environment and/or others. Regarding Rule Four, it is intended that the motivational coping technique “maintain” is an “independent” motivational coping technique wherein for its associated attitudinal trait descriptions to become apparent it does not require a different fundamental categories series of motivational coping techniques and its associated attitudinal trait descriptions to become apparent. However, this series of motivational coping techniques for the fundamental category “confidence” does have a “dependent” series of motivational coping techniques associated to it in the fundamental category “patience” described in iv) below. In other words, in order for the “stress” reaction attitudinal trait descriptions in the motivational coping techniques for the fundamental category “patience” described in iv) below to become apparent to the user during a discussion topic evaluation then it is most likely that the “need” attitudinal trait descriptions corresponding motivational coping techniques in the fundamental category “confidence” is NOT being satisfied or met. Therefore, it is necessary for the user to meet or satisfy the “need” attitudinal trait descriptions using the motivational coping technique “maintain” in the fundamental category “confidence” in order for the “stress” attitudinal trait descriptions from the corresponding “dependent” motivational coping technique in the fundamental category “patience” to not become apparent to the user during the discussion topic evaluation. As a result of meeting or satisfying the “need”, the “stress” is at least reduced or if not eliminated.
Accept (to receive; especially with gladness or approval) is the fourth and last motivational coping technique defined for the fundamental category “confidence” and its associated motivational attribute “need”. Regarding Rule One, to “accept” is intended to be partially descriptive of the fundamental category “confidence” and its associated motivational attribute “need” wherein the attitudinal trait descriptions defined for this fundamental category are considered need descriptors. In other words, to “accept”” something or somebody's “needs” in situations or relationships is critical to developing ones need attitudinal trait descriptors by increasing a user's awareness of where needs may or may not benefit a particular discussion topic related to this motivational coping technique. Regarding Rule Two, it is assumed that the motivational coping technique “accept” achieves an “active-oriented” or higher Δ₂ energy (y-axis) state of mind wherein “to accept” is designed to be more active intended to convey an action or activity. In addition, “active-oriented” motivational coping techniques should be more “advanced-oriented” as compared to the “passive-oriented” techniques wherein if or when achieved it conveys a way to finalize or complete a higher awareness of the fundamental category. Regarding Rule Three, it is intended that the motivational coping technique “accept” is an “attraction” technique wherein it is assumed to convey an intrinsic action response. This type of action intends to encourage a user to absorb information (e.g., “pull-energy”; hence, “attraction”); intending to take an intrinsic action based on information received from the environment and/or others. Regarding Rule Four, it is intended that the motivational coping technique “accept” is an “independent” motivational coping technique wherein for its associated attitudinal trait descriptions to become apparent it does not require a different fundamental categories series of motivational coping techniques and its associated attitudinal trait descriptions to become apparent. However, this series of motivational coping techniques for the fundamental category “confidence” does have a “dependent” series of motivational coping techniques associated to it in the fundamental category “patience” described in iv) below. In other words, in order for the “stress” reaction attitudinal trait descriptions in the motivational coping techniques for the fundamental category “patience” described in iv) below to become apparent to the user during a discussion topic evaluation then it is most likely that the “need” attitudinal trait descriptions corresponding motivational coping techniques in the fundamental category “confidence” is NOT being satisfied or met. Therefore, it is necessary for the user to meet or satisfy the “need” attitudinal trait descriptions using the motivational coping technique “accept” in the fundamental category “confidence” in order for the “stress” attitudinal trait descriptions from the corresponding “dependent” motivational coping technique in the fundamental category “patience” to not become apparent to the user during the discussion topic evaluation. As a result of meeting or satisfying the “need”, the “stress” is at least reduced or if not eliminated.

Patience (Refer to FIG. 2.4.0.0) (Bearing or Enduring Pain, Difficulty, Provocation or Annoyance with Calmness)
b. Comprehend (to take in the meaning, nature, or importance of; grasp) is the first motivational coping technique defined for the fundamental category “patience” and its associated motivational attribute “stress” reactions. Regarding Rule One, to “comprehend” is intended to be partially descriptive of the fundamental category “patience” and its associated motivational attribute “stress” wherein the attitudinal trait descriptions defined for this fundamental category are considered stress descriptors. In other words, to “comprehend” something or somebody's “stress” reaction in situations or relationships is critical to developing the fundamental category “patience”. Regarding Rule Two, it is assumed that the motivational coping technique “comprehend” achieves a “passive-oriented” or lower Δ₁ energy (y-axis) state of mind wherein “to comprehend” achieves more inactivity intended to convey reflection or thought as opposed to encouraging an action. In addition, “to comprehend” achieves a more “beginning-oriented” motivational coping technique conveying an easier way to achieve learning or evolving the fundamental category within a particular discussion topic. Regarding Rule Three, it is intended that the motivational coping technique “comprehend” is a “repulsive” technique wherein it is assumed to convey an extrinsic action response. This type of action intends to encourage a user to initiate an action or make an effort (e.g., “push-energy”; hence “repulsive”); intending to take an action or effort initiated by the user toward the environment and/or others. Regarding Rule Four, it is intended that the motivational coping technique “comprehend” is a “dependent” motivational coping technique wherein for its associated attitudinal trait descriptions to become apparent it does require a different fundamental categories series of motivational coping techniques and its associated attitudinal trait descriptions to NOT be satisfied or nurtured. In other words, a “dependent” series of motivational coping techniques and their attitudinal trait descriptions will only become apparent when the associated “independent” series of motivational coping techniques attitudinal trait descriptors are NOT satisfied or nurtured. Described throughout this invention and for at least one embodiment of this application, the motivational coping techniques including their attitudinal trait descriptions for the fundamental category “patience” are “dependent” on (meaning they will only become apparent) when the corresponding motivational coping techniques and their attitudinal trait descriptions for the fundamental category “confidence” are not satisfied. As a result, when the independent “need” attitudinal trait descriptions from the corresponding motivational coping technique “support” (from the fundamental category “confidence”) is NOT satisfied then the “stress” attitudinal trait descriptions from the motivational coping technique “comprehend” will become apparent. Another way of explaining this concept is by describing that when the motivational attribute “need” from the independent fundamental category “confidence” IS satisfied the motivational attribute “stress” from the dependent fundamental category “patience” will not be apparent.
c. Excuse (to explain in the hope of being forgiven or understood) is the second motivational coping technique defined for the fundamental category “patience” and its associated motivational attribute “stress” reactions. Regarding Rule One, to “excuse” is intended to be partially descriptive of the fundamental category “patience” and its associated motivational attribute “stress” wherein the attitudinal trait descriptions defined for this fundamental category are considered stress descriptors. In other words, to “excuse” something or somebody's “stress” reaction in situations or relationships is critical to developing the fundamental category “patience”. Regarding Rule Two, it is assumed that the motivational coping technique “excuse” achieves a “passive-oriented” or lower Δ₁ energy (y-axis) state of mind wherein “to excuse” achieves more inactivity intended to convey reflection or thought as opposed to encouraging an action. In addition, “to excuse” achieves a more “beginning-oriented” motivational coping technique conveying an easier way to achieve learning or evolving the fundamental category within a particular discussion topic. Regarding Rule Three, it is intended that the motivational coping technique “excuse” is an “attraction” technique wherein it is assumed to convey an intrinsic action response. This type of action intends to encourage a user to absorb information (e.g., “pull-energy”; hence, “attraction”); intending to take an intrinsic action based on information received from the environment and/or others. Regarding Rule Four, it is intended that the motivational coping technique “excuse” is a “dependent” motivational coping technique wherein for its associated attitudinal trait descriptions to become apparent it does require a different fundamental categories series of motivational coping techniques and its associated attitudinal trait descriptions to NOT be satisfied or nurtured. In other words, a “dependent” series of motivational coping techniques and their attitudinal trait descriptions will only become apparent when the associated “independent” series of motivational coping techniques attitudinal trait descriptors are NOT satisfied or nurtured. Described throughout this invention and for at least one embodiment of this application, the motivational coping techniques including their attitudinal trait descriptions for the fundamental category “patience” are “dependent” on (meaning they will only become apparent) when the corresponding motivational coping techniques and their attitudinal trait descriptions for the fundamental category “confidence” are not satisfied. As a result, when the independent “need” attitudinal trait descriptions from the corresponding motivational coping technique “allow” (from the fundamental category “confidence”) is NOT satisfied then the “stress” attitudinal trait descriptions from the motivational coping technique “excuse” will become apparent. Another way of explaining this concept is by describing that when the motivational attribute “need” from the independent fundamental category “confidence” IS satisfied the motivational attribute “stress” from the dependent fundamental category “patience” will not be apparent.
Understand (to perceive and comprehend the nature and significance of; grasp; to know thoroughly) is the third motivational coping technique defined for the fundamental category “patience” and its associated motivational attribute “stress” reactions. Regarding Rule One, to “understand” is intended to be partially descriptive of the fundamental category “patience” and its associated motivational attribute “stress” wherein the attitudinal trait descriptions defined for this fundamental category are considered stress descriptors. In other words, to “understand” something or somebody's “stress” reaction in situations or relationships is critical to developing the fundamental category “patience”. Regarding Rule Two, it is assumed that the motivational coping technique “understand” achieves an “active-oriented” or higher Δ₂ energy (y-axis) state of mind wherein “to understand” is designed to be more active intended to convey an action or activity. In addition, “active-oriented” motivational coping techniques should be more “advanced-oriented” as compared to the “passive-oriented” techniques wherein if or when achieved it conveys a way to finalize or complete a higher awareness of the fundamental category. Regarding Rule Three, it is intended that the motivational coping technique “understand” is a “repulsive” technique wherein it is assumed to convey an extrinsic action response. This type of action intends to encourage a user to initiate an action or make an effort (e.g., “push-energy”; hence “repulsive”); intending to take an action or effort initiated by the user toward the environment and/or others. Regarding Rule Four, it is intended that the motivational coping technique “understand” is a “dependent” motivational coping technique wherein for its associated attitudinal trait descriptions to become apparent it does require a different fundamental categories series of motivational coping techniques and its associated attitudinal trait descriptions to NOT be satisfied or nurtured. In other words, a “dependent” series of motivational coping techniques and their attitudinal trait descriptions will only become apparent when the associated “independent” series of motivational coping techniques attitudinal trait descriptors are NOT satisfied or nurtured. Described throughout this invention and for at least one embodiment of this application, the motivational coping techniques including their attitudinal trait descriptions for the fundamental category “patience” are “dependent” on (meaning they will only become apparent) when the corresponding motivational coping techniques and their attitudinal trait descriptions for the fundamental category “confidence” are not satisfied. As a result, when the independent “need” attitudinal trait descriptions from the corresponding motivational coping technique “maintain” (from the fundamental category “confidence”) is NOT satisfied then the “stress” attitudinal trait descriptions from the motivational coping technique “understand” will become apparent. Another way of explaining this concept is by describing that when the motivational attribute “need” from the independent fundamental category “confidence” IS satisfied the motivational attribute “stress” from the dependent fundamental category “patience” will not be apparent.
Forgive (to excuse for a fault or an offense; pardon; to renounce anger or resentment against) is the fourth motivational coping technique defined for the fundamental category “patience” and its associated motivational attribute “stress” reactions. Regarding Rule One, to “forgive” is intended to be partially descriptive of the fundamental category “patience” and its associated motivational attribute “stress” wherein the attitudinal trait descriptions defined for this fundamental category are considered stress descriptors. In other words, to “forgive” something or somebody's “stress” reaction in situations or relationships is critical to developing the fundamental category “patience”. Regarding Rule Two, it is assumed that the motivational coping technique “forgive” achieves an “active-oriented” or higher Δ₂ energy (y-axis) state of mind wherein “to forgive” is designed to be more active intended to convey an action or activity. In addition, “active-oriented” motivational coping techniques should be more “advanced-oriented” as compared to the “passive-oriented” techniques wherein if or when achieved it conveys a way to finalize or complete a higher awareness of the fundamental category. Regarding Rule Three, it is intended that the motivational coping technique “forgive” is an “attraction” technique wherein it is assumed to convey an intrinsic action response. This type of action intends to encourage a user to absorb information (e.g., “pull-energy”; hence, “attraction”); intending to take an intrinsic action based on information received from the environment and/or others. Regarding Rule Four, it is intended that the motivational coping technique “forgive” is a “dependent” motivational coping technique wherein for its associated attitudinal trait descriptions to become apparent it does require a different fundamental categories series of motivational coping techniques and its associated attitudinal trait descriptions to NOT be satisfied or nurtured. In other words, a “dependent” series of motivational coping techniques and their attitudinal trait descriptions will only become apparent when the associated “independent” series of motivational coping techniques attitudinal trait descriptors are NOT satisfied or nurtured. Described throughout this invention and for at least one embodiment of this application, the motivational coping techniques including their attitudinal trait descriptions for the fundamental category “patience” are “dependent” on (meaning they will only become apparent) when the corresponding motivational coping techniques and their attitudinal trait descriptions for the fundamental category “confidence” are not satisfied. As a result, when the independent “need” attitudinal trait descriptions from the corresponding motivational coping technique “accept” (from the fundamental category “confidence”) is NOT satisfied then the “stress” attitudinal trait descriptions from the motivational coping technique “forgive” will become apparent. Another way of explaining this concept is by describing that when the motivational attribute “need” from the independent fundamental category “confidence” IS satisfied the motivational attribute “stress” from the dependent fundamental category “patience” will not be apparent.

As a result, when properly following the Rules One through Four above in the context of defining a series of motivational coping techniques for a given fundamental category in the Relationship Anatomy Model the users discussion topic evaluation (described in Section B below) will evoke learning and intended to evolve a complete understanding of a given fundamental category. Notice in FIG. 2.1.0.0. through 2.4.0.0., there are three relationship anatomy model structures for each fundamental category that represent three different points in time (z-axis), present time (0), future time (1), and past time (−1). Immediately above, only the present time (0) was used as an example to explain each of the four motivational coping techniques for every fundamental category i) through iv) (e.g., honor, devotion, confidence, and patience). In studying FIG. 2.1.0.0 through 2.4.0.0, you will notice that the present time (0) and past time (−1) motivational coping techniques occupy the same quadrant caches. However, you will also notice that for the future time (1), the motivational coping techniques “switch” quadrant caches wherein the quadrant 1 and quadrant 3 cache motivational coping techniques moves to the quadrant 2 and quadrant 4 cache respectively, and visa versa. This design of the relationship anatomy model is intended to simulate energy through spin and momentum. This energy provides the user opportunities when during a discussion topic evaluation, when the user identifies that they would like to investigate a discussion topic set in the future time (1) they may experience “more-like” attitudinal trait descriptions through “attraction” type motivational coping techniques and “less-like” attitudinal trait descriptions through “repulsive” type motivational coping techniques. Therefore, this design of the relationship anatomy model enables a user to experience every possible outcome between a motivational coping technique and any attitudinal trait description, depending on how they evaluate a discussion topic. Evaluating a discussion topic is described in Section B later in this invention.

10. Discussion Generator
The discussion generator (refer to Table B—Discussion Generator), for a lack of better terminology, is the vehicle that processes and stores the inquiries that are generated back to the user in Table C—User Presentation. The discussion generator, in one way or another, utilizes all of the elements described in Section A above (1 through 9) to construct all of the possible inquiries which are generated back to the user for a user's discussion topic evaluation which is described in Section B below. In other words, depending on the users discussion topic evaluation and the discussion number selected from the user presentation, the discussion generator becomes the lookup table for the user presentation wherein the discussion generator assembles and constructs the inquiries while utilizing information from the description construct table (e.g., the users intake process of choosing which attitudinal trait descriptions are “more-like” or, by default, “less-like” themselves) and the user presentation to generate, for each fundamental category, the inquiries back to the user for the user to investigate their discussion topic.
For the purpose of simplifying this explanation refer to the fundamental category “honor” in Table B—Discussion Generator and refer to the relationship anatomy model for this fundamental category in FIG. 2.1.0.0. First, look at Table—B—Discussion Generator. Notice the column labeled “state of mind”. There are twelve states of mind references, A through L. For each state of mind there are three energy field coordinates (see the field called energy field coordinates) which contain either a value of 1, 0, or −1. For example, look at state of mind labeled “A” for the fundamental category “honor”. The first coordinate is the relative bond or x-axis coordinate with a value equal to 1. The second coordinate is the energy or y-axis coordinate with a value equal to 0. And the third coordinate is the time or z-axis coordinate with a value equal to 0. These coordinates are concatenated together in the field called concatenate energy field coordinates (e.g., 100). Next, this number is converted to represent a wave function and stored in the field called wave function |ψ> (e.g., |100>). Before explaining addition fields in the Table B—Discussion Generator first refer to the relationship anatomy model in FIG. 2.1.0.0. Locate the wave function |100>. It is located in the quadrant 1 cache (lower right quadrant cache) on the present (0) time z-axis. Notice that there are twelve wave functions for this fundamental category (i.e., |100>, |000>, |110>, |010>, |001>, |101>, |011>, |111>, −|101>, −|001>, −|111>, −|011>). Each of these wave functions are coordinates defined for each fundamental category in the energy field coordinates stored in the Table B—Discussion Generator described above and are described in more detail in subsection 8b above). You will also notice that these coordinates are repeated in the same pattern for each fundamental category. Now it is established how the relationship anatomy model, at least in part, is a direct design element in the Table B—Discussion Generator. Next, the Table B—Discussion Generator defines a field called the charge field coordinate. In order to fully appreciate the following it may be beneficial to review the charge fields (attitudinal trait descriptions) described in subsection 4 above. For each state of mind in every fundamental category this field is either defined as “more” or “less” wherein “more” represents attitudinal trait descriptions “more-like” the user and “less” represents attitudinal trait descriptions “less-like” the user. This field doesn't store the actual attitudinal trait description used in the inquiry but rather identifies the coordinate in the relationship anatomy model which shows whether the wave function or concatenated energy field coordinates represent a user's “more-like” or “less-like” attitudinal trait descriptions. For instance, refer back to state of mind “A” in the Table B—Discussion Generator. You'll notice that for the wave function |100> the charge field coordinate is “more”. Now, refer back to the relationship anatomy model in FIG. 2.1.0.0. Again, locate the wave function |100>. Notice the legend at the top of the figure wherein a grey box is labeled “less-like descriptions” and a white box labeled “more-like descriptions”. Notice that the quadrant cache where the wave function is |100>, this quadrant cache represents “more-like descriptions” (e.g., the white box). Therefore, state of mind “A” will always represent attitudinal trait descriptions that are “more-like” the user. Yet another aspect of the Table B—Discussion Generator is that for each fundamental category the motivational coping techniques are defined by the facilitator and the relationship anatomy model is used as the guide to defining these techniques. In order to fully appreciate the following it may be beneficial to review the motivational coping techniques described in subsection 9 above. Notice the field called motivational coping technique inquiry component in Table B—Discussion Generator. Continuing our example, refer to state of mind “A”. You'll notice that the motivational coping technique defined for this state of mind in the fundamental category “honor” is “observe”. Next, refer back to the relationship anatomy model in FIG. 2.1.0.0 and locate the wave function |100>. You'll notice the motivational coping technique defined for this wave function in the fundamental category “honor” is “observe”. In addition, according to the rules defined for motivational coping techniques in subsection 9 above, you should notice that the motivational coping technique “observe” is representative of the same coordinates, with the exception of the charge field coordinate (attitudinal trait descriptions), for each time (e.g., z-axis) coordinate. In other words, “observe” will always represent a “repulsive” or push energy and it will always represent a “passive-oriented” or low energy motivational coping technique. However, you will notice that in the present (0) and past (−1) time measures, “observe” represents a users “more-like” attitudinal trait descriptions. And in the future (1) time measure, “observe” represents a user's “less-like” attitudinal trait descriptions (refer to subsection 8b for further explanation of this relationship anatomy model concept). To this point, all of the fields described above are represented in the relationship anatomy model in FIG. 2.0.0.0 through 2.4.0.0. However, it is yet another aspect of the discussion generator to construct the inquiries that are generated the back to the user during a discussion topic evaluation wherein these inquiries are constructed by combining different components called inquiry components. One component, the motivational coping technique inquiry component, is described immediate above. However, this is just one of other components necessary to construct an inquiry. Another component of the inquiry is called the energy level inquiry component. This is the beginning of the inquiry. Continuing the example described in this section, refer back to the state of mind “A” for the fundamental category “honor”. Locate the energy level inquiry component. You'll notice is says “What do you”. For inquiries where the motivational coping technique is “passive-oriented” (e.g., “observe” is “passive-oriented”) the energy level inquiry component will begin with “What . . . ” which assumes a user to be more inactive in their pursuit to discover answers in their investigation of the discussion topic. Depending on how a user's discussion topic evaluation of a discussion topic, the energy level inquiry component may change according to “active-oriented” vs “passive-oriented” motivational coping techniques and which time measure value is defined for the state of mind (for a more detailed description of this concept refer to subsection 5 Energy Fields above). Next, the prefix inquiry component is defined in Table B—Discussion Generator. In brief, for inquiries that begin with “What” the prefix inquiry component is “in”. Otherwise, this field is left blank (refer to prefix inquiry component in terms and definitions above). You'll notice in our example using the state of mind “A” for the fundamental category “honor”, the prefix inquiry component is “in”. The next inquiry component is called the motivational attribute inquiry component. For a deeper understanding of this component, refer to subsection 2 called motivational attribute and see a detailed definition of this component in the terms and definitions section above. At least for one embodiment of the present invention, for each fundamental category there is one motivational attribute. Continuing to use our example, in the state of mind “A” for the fundamental category “honor” the motivational attribute is “strength”. Notice in the motivational attribute inquiry component field for this state of mind, the text reads “your strength of being”. It may be important to note that for charge fields (i.e., attitudinal trait descriptions) that are “more-like” the user the phrase in the motivational attribute inquiry component will begin with “your”, otherwise, for attitudinal trait descriptions “less-like” the user, the phrase in the motivational attribute inquiry component will begin with “others”. So far, the inquiry that has been constructed in our example where the state of mind is equal to “A” for the fundamental category “honor” reads—“What do you observe in your strength of being . . . ” At this point it is important to note that for the components described immediately above in this subsection, the values defined for each are fixed and permanent, designed and defined by the facilitator.
There is yet one more component defined in the Table B—Discussion Generator that represents the final or last component needed before constructing or combining all of the components together into an inquiry that can be generated back to the user during a user's discussion topic evaluation. This component is called the description inquiry component. Continuing our example from above in the state of mind field equal to “A” for the fundamental category “honor”, find the description inquiry component field in the Table B—Discussion Generator. You will find two subfields under the description inquiry component field called “more like description inquiry component” and “less like description inquiry component”. You should notice that for each state of mind and fundamental category the “more like description inquiry component” entry (e.g., friendly and easy to know) is the same throughout and the “less like description inquiry component” entry (e.g., able to work well alone) is the same throughout. The calculation made for these subfields are based on the field in the Table B—Discussion Generator called the fundamental category description choice. Notice that in our example for the state of mind “A” in the fundamental category “honor” the fundamental category description choice is “b” (it is important to point out that “b” is represented throughout the fundamental category “honor”; the explanation for this will become more apparent in the following). Note that for this calculation to work, during the user's discussion topic evaluation, a discussion number between 1 and 11 is determined on the user presentation interface (e.g., discussion number equals 1; see FIG. 3.0.0.0). Combining the fundamental category and the discussion number (e.g., “honor” & 1; honor1), the fundamental category description choice field uses this value (e.g., “honor1”) to look up the fundamental category description choice value (e.g., “b”) in a field called the description choice evaluation procedure in the Table A—Description Construct Table. Now, refer to Table A—Description Construct Table. Under a field called “DOUBLEKEY”, find the value “honor1”. You will notice that the value in the description choice evaluation procedure field equals “b”. It is also important at this point to notice that in the Table A—Description Construct Table the attitudinal trait descriptions for “honor1” wherein the fundamental category description choice value equals “b” are “friendly and easy to know” for the field called More Like Descriptions and “able to work well alone” for the field called Less Like Descriptions. These attitudinal trait descriptions were defined to be “more-like” and “less-like” the user during the user's intake process wherein the user chose “b”, “friendly and easy to know” as “more-like” themselves (see Description B field in the Table A—Description Construct Table for the DOULBLEKEY field equal to “honor1”). Now, refer back to the fields that we are currently explaining in Table B—Discussion Generator called the more like description inquiry component and less like description inquiry component. Referring back to our example in Table B—Discussion Generator wherein the state of mind value is “A” for the fundamental category “honor”, the more like description inquiry component field builds the value (e.g., “honor1”) by combining the fundamental category (e.g., “honor”) and the discussion number from the user presentation (e.g., 1) that is used to look up the more like description value (e.g., friendly and easy to know) in the Table A—Description Construct Table. This value (e.g., “honor1”) is also used to look up the less like description value (e.g., able to work well alone) in the Table A—Description Construct Table.
As a result, for our ongoing example in Table B—Discussion Generator wherein the state of mind equals “A” for the fundamental category “honor”, the last component to build for every state of mind is the combined inquiry. The combined inquiry combines all of the inquiry components described above in to one inquiry for each state of mind that can be, depending on the user's discussion topic evaluation, generated back to the user for the user to use to investigate a discussion topic. The combined inquiry combines the energy level inquiry component (e.g., What do you), the motivational coping technique inquiry component (e.g., “observe”), the prefix inquiry component (e.g., “in”), and the motivational attribute inquiry component (e.g., your strength of being). As stated above, so far, this is concatenated as “What do you observe in your strength of being . . . ”. The last component is either the more like description inquiry component or the less like description inquiry component. When combining the inquiry, the combined inquiry field either utilizes the value from the more like description inquiry component field or the value from the less like description inquiry component field. For each state of mind, if the charge field coordinate equals “more” then the combined inquiry field concatenates the value from the more like description inquiry component field (e.g., friendly and easy to know). Otherwise, it will concatenate the value from the less like description inquiry component field (e.g., able to work well alone). Continuing with our example, since the value is equal to “more” in the charge field coordinate field in Table B—Discussion Generator in the state of mind field equal to “A” for the fundamental category “honor”, the final combined inquiry reads:

“What do you observe in your strength of being friendly and easy to know?”

Section B—User Presentation

Section B describes the user's presentation of the invention as it relates to how a user interacts with this system in everyday situations, relationships, or aspects of general life (e.g., discussion topics). Accordingly, it is an aspect of the Table B—Discussion Generator and the B—DISCUSSION GENERATOR 2 component in FIG. 1.0.0.0. and the MOTIVTIONAL COPING TECHNIQUE CONSTRUCTOR 24 component in FIG. 1.2.0.0, DESCRIPTION INQUIRY COMPONENT 35 component in FIG. 1.2.2.0, and the COMBINED INQUIRY 30 component in FIGS. 1.2.0.0 and 1.2.2.0 of the present invention to generate inquiries, within a context personalized both to the user's attitudinal trait descriptions (e.g., behaviors or attitudes) and to the user specified discussion topic within the Table C—User Presentation and the C—USER PRESENTATION 3 component in FIG. 1.0.0.0. and in FIG. 1.3.0.0, and the GENERATE DISCUSSION TOPIC INQURIES 54 in FIG. 1.3.1.0, so that the user can investigate, reflect upon, and/or gain greater insight into his/her perceptions of the discussion topic while relating to the attitudinal trait descriptions via the pre-defined motivational coping techniques for each pre-defined fundamental category described in subsections 1, 3, 4 and 9 above. In other words, the discussion generator generates, for each of one or more predetermined fundamental categories and their motivational coping techniques, inquiries directed to assisting the user to cope with and/or gain greater insight into his/her a “most like” or “least like” attitudinal trait descriptions as it relates to the discussion topic, wherein these inquiries are phrased in a manner that is believed to cause the user, when reflecting on the inquiries, to examine the discussion topic from a particular (e.g., user motivated) perspective from which the user might not otherwise readily be able to recognize. Put more simply, asking the right inquiries can help users discover some personal answers to an issue or situation (e.g., a discussion topic).

It is another aspect of this invention to define a user specified discussion topic, evaluate the discussion topic, and generate inquiries back to the user for a users consideration in a manner that is customized to the user. The sequential process of the C—USER PRESENTATION 3 in FIG. 1.3.0.0. is described in 1 through 3 below (also see FIG. 3.0.0.0):

1. Discussion Topic
Before a user evaluates a Discussion Topic in the Table A—User Presentation and the C—USER PRESENTATION 3 component in FIG. 1.3.0.0, a user must define a discussion topic to evaluate in which a user would like to investigate. First, in the SELECT DISCUSSION TOPIC 42 component in FIG. 1.3.0.0 and in the user interface in FIG. 3.0.0.0 the user thinks of a discussion topic based on six general topics that include, but not limited to, family, friends, faith, finances, work, and home. Once the user thinks of a discussion topic, the user simply selects a general discussion topic (e.g., work) that best relates to the discussion topic the user wishes to investigate.

2. Discussion Topic Evaluation Statements

In Table C—User presentation and in the DISCUSSION TOPIC EVAUATION STATEMENTS 2 component in FIG. 1.3.0.0 and in the user interface in FIG. 3.0.0.0, the discussion topic selected above in the first step is automatically generated as part of three statements generated wherein the user answers each of the three statements while focusing on the discussion topic the user wishes to investigate. Each of the three statements a), b), and c) respectively relate to the x-axis, y-axis, and the z-axis in relationship anatomy model and in Table B—Discussion Generator under the energy field coordinates.

a) Discussion Topic Evaluation Statement a) (x-axis)
Shown in the users interface in FIG. 3.0.0.0, statement a) focuses its design directly on the relative bond axis (x-axis) wherein the statement for the user to answer is “I feel stable when it comes to my work” (“work” is the selected discussion topic from step 1 above wherein it is automatically inserted into the statement). The user has two choices, “yes” or “no” (e.g., “yes”=1 and “no”=0—the first digit of the triplet discussed in subsection 8b in section A above; primarily Wave Functions |ψ>), wherein when the user answers “no” (meaning “no” represents a 0 value) this will relate directly to the “attraction” type of motivational coping techniques (i.e., intrinsic-oriented; pull energy) for each fundamental category discussed in subsection 1 above (e.g., “allow” or “accept” shown in FIG. 2.3.0.0 for the fundamental category “confidence”) and when the user answers “yes” this will relate directly to the “repulsive” type of motivational coping techniques (i.e., extrinsic-oriented; push energy) for each fundamental category (e.g., “support” or “maintain” motivational coping techniques shown in FIG. 2.3.0.0 for the fundamental category “confidence”). It is assumed that when the user feels stable (i.e., answering “yes” to this statement) when it comes to a given discussion topic, they will feel more inclined, more comfortable, to initiate extrinsic-oriented (e.g., “repulsive” motivational coping techniques represent extrinsic-oriented; push energy) motivational coping techniques (e.g., “support” or “maintain”). On the contrary, it is assumed that when a user does NOT feel stable (i.e., answering “no” to this statement) when it comes to a given discussion topic, they will feel more inclined to initiate intrinsic-oriented (i.e., more reflection oriented) (e.g., “attraction” motivational coping techniques represent intrinsic-oriented; pull energy) motivational coping techniques (e.g., “allow” or “accept”).

b) Discussion Topic Evaluation Statement b) (y-axis)
Also, shown in the user's interface in FIG. 3.0.0.0, statement b) focuses its design directly on the energy axis (y-axis) wherein the statement for the user to answer is “I feel enthusiastic when it comes to my work” (“work” is the selected discussion topic from step 1 above wherein it is automatically inserted into the statement). The user has two choices, “yes” or “no” (e.g., “yes”=1 and “no”=0—the second digit of the triplet discussed in subsection 8 in section A above; primarily Wave Functions |ψ>), wherein when the user answers “no” (meaning “no” represents a 0 value) this will relate directly to the “passive-oriented” or beginning-oriented type of motivational coping techniques (i.e., initiating less effort) for each fundamental category discussed in subsection 1 above (e.g., “support” or “allow” shown in FIG. 2.3.0.0 for the fundamental category “confidence”) and when the user answers “yes” this will relate directly to the “active-oriented” and advanced-oriented type of motivational coping techniques (e.g., initiating more effort) for each fundamental category (e.g., “maintain” or “ accept” motivational coping techniques shown in FIG. 2.3.0.0 for the fundamental category “confidence”). It is assumed that when the user feels enthusiastic (i.e., answering “yes” to this statement) when it comes to a given discussion topic, they will feel more inclined, more comfortable, to initiate more effort (e.g., “active-oriented” motivational coping techniques represent initiating more effort) motivational coping techniques (e.g., “maintain” or “accept”). On the contrary, it is assumed that when a user does NOT feel stable (i.e., answering “no” to this statement) when it comes to a given discussion topic, they will feel more inclined to initiate less effort (i.e., more reflection oriented) (e.g., “passive-oriented” motivational coping techniques represent less effort) motivational coping techniques (e.g., “support” or “allow”).

c) Discussion Topic Evaluation Statement c) (z-axis)
Last, shown in the user's interface in FIG. 3.0.0.0, statement c) focuses its design directly on the time axis (z-axis) wherein the statement for the user to answer is “I feel confident when it comes to my work” (“work” is the selected discussion topic from step 1 above wherein it is automatically inserted into the statement). The user has three choices, “yes” or “no” or “used to be” (e.g., “yes”=1 and “no”=0 and “used to be”=−1—the third digit of the triplet discussed in subsection 8 in section A above; primarily Wave Functions |ψ>), wherein, first, when the user answers “no” to statement c) (meaning “no” represents a 0 value in the time z-axis energy field coordinates field) it relates directly to placing the user in the present tense wherein the inquiry generated back to the user is designed to begin with “What do you . . . ” (for passive-oriented selected motivational coping techniques) or “How do you . . . ” (for active-oriented selected motivational coping techniques). The timeframe or tense portion of the inquiry generated back to the user is designed in Table B—Discussion Generator in the field called the energy level inquiry component. Here, when the time measure value equals 0, it is assumed by the inventor that the word “do” in the context of “What do you . . . ” or “How do you . . . ” places the user in the present state of mind wherein the user should investigate the discussion topic and how it relates to them now. It is believed by the inventor that when the user answers “no” to “I feel confident when it comes to my work”, it is generally optimal for the user to deal with situations or issues in the present tense, confident that it is healthier to focus on issues in the moment. However, it could be argued, but believed to be less than optimal by the inventor, that a lack of confidence or trust in something that should be dealt with in the past tense. On the other hand, as described later, if the user chooses “used to be” confident, then it should be safe to assume that the user is not confident now (i.e., in the present) but was some time ago, therefore, designing the inquiry to place the user's state of mind in a timeframe (i.e., in the past) when they were confident and to direct their state of mind on actions, thoughts, and activities they were doing in the past that made them feel confident as it pertains to the discussion topic. This is believed by the inventor to be a more effective way of raising confidence from the past to foster confidence in the present. Second, when the user answers “yes” to statement c) (meaning “yes” represents a value of 1 in the time z-axis energy field coordinates field) it relates directly to placing the user in the future tense wherein the inquiry generated back to the user is designed to begin with “What will you . . . ” (for passive-oriented selected motivational coping techniques) or “How will you . . . ” (for active-oriented selected motivational coping techniques). The timeframe or tense portion of the inquiry generated back to the user is designed in Table B—Discussion Generator in the field called the energy level inquiry component. Here, when the time measure value equals 1, it is assumed by the inventor that the word “will” in the context of “What will you . . . ” or “How will you . . . ” places the user in the future state of mind wherein the user should investigate the discussion topic and how it relates to them in the future. It is believed by the inventor that when the user answers “yes” to “I feel confident when it comes to my work”, it is generally optimal for the user to “look forward” in the future tense, confident that when a user is feeling confident regarding a discussion topic their outlook is ripe for clearer decision making. Third, when the user answers “used to be” to statement c) (meaning “used to be” represents a −1 value in the time z-axis energy field coordinates field) it relates directly to placing the user in the past tense wherein the inquiry generated back to the user is designed to begin with “What did you . . . ” (for passive-oriented selected motivational coping techniques) or “How did you . . . ” (for active-oriented selected motivational coping techniques). The timeframe or tense portion of the inquiry generated back to the user is designed in Table B—Discussion Generator in the field called the energy level inquiry component. Here, when the time measure value equals −1, it is assumed by the inventor that the word “did” in the context of “What did you . . . ” or “How did you . . . ” places the user in the past state of mind wherein the user should investigate the discussion topic and how it relates to them in the past. It is believed by the inventor that when the user answers “used to be” to “I feel confident when it comes to my work”, it is generally optimal for the user to look back on situations or issues in the past tense, confident that it is healthier to focus on issues that may be difficult now and how a user may adjust their perception of the discussion topic as it relates to their confidence by directing their focus on issues or situations where they felt a higher confidence level. This is believed by the inventor to be a more effective way of raising confidence from the past to foster confidence in the present.

3. Discussion Topic Inquiries

In the Table C—User presentation and in the B—DISCUSSION GENERATOR 2 component in FIG. 1.2.0.0 and in the user interface in FIG. 3.0.0.0, the discussion topic selected along with the user answering the three discussion topic evaluation statements (i.e., a triplet) described immediately above generates inquiries, one for each fundamental category, from Table B—Discussion Generator. The answers, or more specifically the values (0, 1, or −1), from the three discussion topic evaluation statements which are chosen by the user for a discussion topic are concatenated together in order (i.e., a), b), and c)) and form a triplet measure which is simply converted into a wave function by assigning it as a wave function |ψ>. Notice in FIG. 3.0.0.0 immediately above the section 3) Discussion Topic Inquiries and to the right of the discussion number (#), you'll notice a three digit number (e.g., 011; this is only shown in the figure to simplify how inquiries are generated from Table B—Discussion Generator). Notice, these three digits represent, in order, the answers, or values generated as a result of the answers provided by the user of the discussion topic evaluation statements. Now notice that there are four inquiries listed in FIG. 3.0.0.0 in section 3) Discussion Topic Inquiries wherein (from top to bottom) the first inquiry represents the fundamental category “honor”, the second inquiry represents the fundamental category “devotion”, the third inquiry represents the fundamental category “confidence” and the fourth inquiry represents the fundamental category “patience”. In this example, for each of the four inquiries, the number 011 (i.e., |011>) is used to look up each inquiry in the Table B—Discussion Generator within the concatenate energy field coordinates field. Also notice that in FIG. 3.0.0.0 the discussion number (#) is equal to 1. As described in subsection 4 of Section A above, Charge Fields (Attitudinal Trait Descriptions), during the intake, the user chooses between 11, but not limited to 11, pairs of attitudinal trait descriptions for each fundamental category that is “most-like” themselves, and the remaining descriptions defaults to being “less-like” themselves. There are 11 pairs of attitudinal trait descriptions for the user to choose from for each fundamental category, numbered from 1 to 11 (see Table A—Description Construct Table in the discussion number field). This number is denoted as the discussion number (#). Changing the discussion number (#) (e.g., by clicking “next discussion” shown in FIG. 3.0.0.0) will not change the answers given by the user for the discussion topic evaluation statements which determine the “passive-oriented” or “active-oriented” state of mind, the reference to attitudinal trait descriptions being “more-like” or “less-like” themselves, or the motivational coping technique for each fundamental category, but it will change the attitudinal trait description according to the discussion number listed in the user interface. So, even though this invention could be designed to present the user with a plurality of attitudinal trait descriptions for a discussion topic, this embodiment of the present invention has simplified the process to only show one attitudinal trait description for each inquiry at a time. During a discussion topic evaluation, a user may choose a different discussion number (#) to evaluate wherein the user may sift through a plurality of attitudinal trait descriptions (e.g., by continuing to click the “next discussion” button) until they discover the attitudinal trait descriptions that make the most sense to their investigation of the discussion topic. So, in order to simplify the explanation below of how the inquiries are generated back to the user during a discussion topic evaluation, as shown in FIG. 3.0.0.0, for each fundamental category the attitudinal trait description that is selected will be from discussion number 11. The following describes a step by step process for looking up each of the four inquires shown in the example provided from FIG. 3.0.0.0. In addition, refer to Table B—Discussion Generator and Table A—Description Construct Table. Referring to FIG. 3.0.0.0:
a. The first inquiry generated from the discussion generator relates to the fundamental category “honor”. The triplet 011 or wave function |011>, is looked up in Table B—Discussion Generator in the concatenate energy field coordinates field within the fundamental category “honor”. The triplet 011 is found in the field state of mind labeled “G”. Next, the inquiry associated to this triplet is found in the field called combined inquiry. Built into the inquiry, the discussion generator builds the attitudinal trait description into the inquiry from the description construct table for the fundamental category “honor” that match the discussion number (e.g., 11) listed in the user interface. As a result, the inquiry generated back to the user is (see FIG. 3.0.0.0):
   “How will you respect your strength of being able to handle ambiguous situations well?”
   To which the user might respond:
   I will take my time and put more effort toward listening to others input and take more into consideration before reacting to a situation or an issue. I believe this will enable me to use this strength more effectively.
   NOTE: Notice that the location of the “respect” motivational coping technique in quadrant 3 cache of the “future” time measure (z-axis) in FIG. 2.1.0.0 (e.g., wave function |011>), you will find the attitudinal trait description, “able to handle ambiguous situations well”, displayed in Table B—Discussion Generator within the “honor” fundamental category under the label “more like description inquiry component. (a detailed explanation of how the attitudinal trait descriptions are selected and displayed in the Appending B—Discussion Generator are explained in the subsection 10 of Section A) Also, as discussed in subsection number 3 & 4 of section A above, note that attitudinal trait descriptions are “selected” and “locked in” by the user as a portion of the intake process to be classified as “more like descriptions” which are “more like” the user or “less like descriptions” which are “less like” the user (refer to substructure 1, Charge Fields, in FIG. 2.0.0.0). This logic applies the same throughout this subsection and further explanation is provided throughout this application.
b. The second inquiry generated from the discussion generator relates to the fundamental category “devotion”. The triplet 011 or wave function |011>, is looked up in Table B—Discussion Generator in the concatenate energy field coordinates field within the fundamental category “devotion”. The triplet 011 is found in the field state of mind labeled “G”. Next, the inquiry associated to this triplet is found in the field called combined inquiry. Built into the inquiry, the discussion generator builds the attitudinal trait description into the inquiry from the description construct table for the fundamental category “devotion” that match the discussion number (e.g., 11) listed in the user interface. As a result, the inquiry generated back to the user is (see FIG. 3.0.0.0):
   “How will you discover your interest in using imagination and intuitiveness?”
   To which the user might respond:
   Fortunately, I have had many opportunities to be imaginative in my job. I believe I could increase this interest if I direct all of my focus on finding solutions to other's issues, staying away from concerning myself with issues that don't serve anyone.
c. The third inquiry generated from the discussion generator relates to the fundamental category “confidence”. Remember, “confidence” is an independent type of fundamental category which has a “dependent” fundamental category associated to it (e.g., “patience” described immediately below in d. and in Rule 4 above and in subsection 3 in Section A). The triplet 011 is found in the field state of mind labeled “G”. Next, the inquiry associated to this triplet is found in the field called combined inquiry. Built into the inquiry, the discussion generator builds the attitudinal trait description into the inquiry from the description construct table for the fundamental category “confidence” that match the discussion number (e.g., 11) listed in the user interface. As a result, the inquiry generated back to the user is (see FIG. 3.0.0.0):
   “How will you accept your need for plenty of time to make complex decisions?”
   To which the user might respond:
   Overtime, I have learned that this feels like a weakness to me in an environment that thrives on rushing things. This seems to be challenging in today's work environments. I know that I have difficulty in rushing decisions. Sometimes, I feel like I am in damage control some of the time with indecision in some circumstances. Although I believe this to be very true about myself, I continue to struggle with completely accepting this about myself, always thinking it might change, but it won't. I continue to communicate to others that I need time to make decisions, but I believe its only had a little effect. However, I will continue to communicate this to my work environment.
d. The forth inquiry generated from the discussion generator relates to the fundamental category “patience”. Remember, “patience” is a “dependent” type of fundamental category wherein its associated independent fundamental category is “confidence” described immediately above in c. In other words, this invention is designed for dependent fundamental categories that when the independent fundamental category's attitudinal trait descriptions are satisfied then the dependent fundamental category's attitudinal trait descriptions are not apparent. So, if the inquiry from c. immediately above was always satisfied (e.g., How will you accept your need for plenty of time to make complex decisions?”) then the inquiry described below would never be apparent. Otherwise, they will become apparent. Moving on, the triplet 011 or wave function |011>, is looked up in Table B—Discussion Generator in the concatenate energy field coordinates field within the fundamental category “patience”. The triplet 011 is found in the field state of mind labeled “G”. Next, the inquiry associated to this triplet is found in the field called combined inquiry. Built into the inquiry, the discussion generator builds the attitudinal trait description into the inquiry from the description construct table for the fundamental category “patience” that match the discussion number (e.g., 11) listed in the user interface. As a result, the inquiry generated back to the user is (see FIG. 3.0.0.0):
   “Hint: forgive your stress toward having indecision when pressured?”
   To which the user might respond:
   I believe that I feel some stress when pressured to make a decision. If I do than I find later that I may regret the decision and sometimes change my mind. This is stressful also, so, I believe that if I focus on my need (discussed in c. above for the fundamental category “confidence”) for needing “plenty of time to make complex decisions” then, hopefully, this will reduce my stress of having indecision when I'm pressured to make a decision.

### BRIEF DESCRIPTION OF THE COMPONENTS IN THE BLOCK DIAGRAM OF THE INVENTION

(Referring to FIGS. 1.0.0.0 through FIG. 1.3.1.0)

A—DESCRIPTION CONSTRUCT TABLE 1

This component organizes and stores all of the attitudinal trait description data that is defined by the facilitator and is used as an interface to the user wherein the user selects the attitudinal trait descriptions that are “more-like” themselves. Refer to Table C—Description Construct Table.

B—DISCUSSION GENERATOR 2

This component stores the information that is used to generate all of the possible inquiries for each state of mind, including the motivational coping techniques. The information in this component is based on the design of the relationship anatomy model (see FIG. 2.0.0.0 through 2.4.0.0). Refer to Table B—Discussion Generator.

C—USER PRESENTATION 3

This component aids the user of the system by providing an interface wherein the user defines a discussion topic, answers three discussion topic evaluation statements, and then based on the user's answers generates optimum inquiries from the discussion generator to which the user uses to investigate their discussion topic.

DEFINE FIRST FUNDAMENTAL CATEGORY 4

This component is used by the facilitator to define the Fundamental Categories (e.g., confidence) of the system.

MOTIVATIONAL ATTRIBUTE 5

This component is used by the facilitator to define the Motivational Attributes (e.g., need) of the system.

DESCRIPTION TYPE 6

This component is used by the facilitator to define the Description Type (e.g., independent or dependent) of each Fundamental Category.

DESCRIPTION COMPONENT 7

This component is used by the facilitator to define the Description Component (e.g., Acceptance) of each pair of Attitudinal Trait Descriptions.

DESCRIPTION A 8 & DESCRIPTION B 9

These components are used by the facilitator to define and store each pair (i.e., Description A & Description B) of Attitudinal Trait Descriptions.

DESCRIPTION NUMBER 10

This component numbers each pair of Attitudinal Trait Descriptions (e.g., 1 through 11).

USER DESCRIPTION CHOICE 11

This component is used by the user wherein for each pair of Attitudinal Trait Descriptions the user chooses the description that is “more-like” themselves. For each pair of Attitudinal Trait Descriptions the description that is not chosen defaults to “less-like” the user.

USER CHOOSES DESCRIPTION A 12

This component represents the description wherein the user chooses Description A to be “more-like” themselves.

USER CHOOSES DESCRIPTION B 13

This component represents the description wherein the user chooses Description B to be “more-like” themselves.

MORE LIKE DESCRIPTION 14

This component represents a list of the user's “more-like” Attitudinal Trait Descriptions.

LESS LIKE DESCRIPTION 15

This component represents a list of the user's “less-like” Attitudinal Trait Descriptions.

NEXT DESCRIPTION COMPONENT 16

This component is used by the facilitator to define the next Description Component (e.g., Acceptance) the next pair of Attitudinal Trait Descriptions.

DEFINE NEXT FUNDAMENTAL CATEGORY 17

This component is used by the facilitator to define the next Fundamental Category (e.g., confidence) in the system.

DEFINE FIRST FUNDAMENTAL CATEGORY 18

This component is used by the facilitator to find and record in the discussion generator the first Fundamental Category defined (e.g., honor) in the system from the A—DESCRIPTION CONSTRUCT TABLE 1 component. This is the first step of the B—DISCUSSION GENERATOR 2 component described above.

DEFINE FIRST STATE OF MIND 19

This component is used by the facilitator to classify each state of mind (e.g., A through L) for every Fundamental Category defined (e.g., honor) in the system.

ENERGY FIELD COORDINATES (X, Y, Z) 20

This component is used by the facilitator wherein the facilitator defines a triplet of coordinates (e.g., 011) for each state of mind classified (e.g., A through L) within every Fundamental Category defined (e.g., honor) in the system. This triplet represents a position in the relationship anatomy model which represents a plurality of predefined variables necessary to generate inquires back to the user during a user's discussion topic evaluation. This triplet is also converted into a wave function wherein a wave function (e.g., |011>) represents energy and momentum in order to demonstrate how the evolution of the motivational coping techniques, which are defined for each wave function, accelerate the learning of every fundamental category (e.g., confidence).

RELATIVE BOND (X-AXIS) 21

This component is used by the facilitator wherein the facilitator defines the first in a triplet of coordinates (e.g., “0”11) for each state of mind within every fundamental category. The first measure of the triplet represents a position in the relationship anatomy wherein a motivational coping technique either represents a “repulsive” or extrinsic action (e.g., push energy type) motivational coping technique or an “attraction” or intrinsic action (e.g., pull energy type) motivational coping technique. “Repulsive” motivational coping techniques represent a downward momentum (similar to that of an electron in a subatomic particle system) and an “attraction” motivational coping technique represents an upward momentum (similar to that of an electron in a subatomic particle system).

ENERGY (Y-AXIS) 22

This component is used by the facilitator wherein the facilitator defines the second in a triplet of coordinates (e.g., 0“1”1) for each state of mind within every fundamental category. The second measure of the triplet represents a position in the relationship anatomy wherein a motivational coping technique either represents a “passive-oriented” (e.g., initiating less energy) motivational coping technique or an “action-oriented” (e.g., initiating more energy) motivational coping technique. Passive-oriented motivational coping techniques are considers “beginning-oriented” as compared to their associated action-oriented motivational coping techniques wherein action-oriented motivational coping techniques are more “advanced-oriented” as compared to their associated passive-oriented motivational coping techniques.

TIME (Z-AXIS) 23

This component is used by the facilitator wherein the facilitator defines the third in a triplet of coordinates (e.g., 01“1”) for each state of mind within every fundamental category. The third measure of the triplet represents a position in time (z-axis) wherein time is represented for every anatomy relationship model module defined in FIG. 2.1.0.0 through 2.4.0.0 as either “present time”, “future time”, or “past time. This coordinate is used to generate inquiries back the user wherein the inquiry is designed to direct the users mind set to either be in the present, future, or the past.

MOTIVATIONAL COPING TECH CONSTRUCTOR 24

This component is used by the facilitator to defines ways in which one would cope in developing or evolving each fundamental category (e.g., confidence) for its corresponding motivational attribute in relation to each set of attitudinal trait descriptions. Two or more motivational coping techniques are defined for each fundamental category. In one embodiment of this application, four motivational coping techniques are defined for each fundamental category (e.g., confidence). The requirements that go into defining each of the motivational coping techniques are described above in the components RELATIVE BOND (X-AXIS) 21, ENERGY (Y-AXIS) 22 and in subsection 9 of Section A (e.g., Rules a) through d)) in the Detailed Description Of the Invention.

APPLY MOTIVATIONAL COPING TECH RULES 25

This component is used by the facilitator to aid the facilitator in defining each of the motivational coping techniques for each fundamental category (e.g., confidence) and for its corresponding motivational attribute in relation to each set of attitudinal trait descriptions. Refer to subsection 9 of Section A (e.g., Rules a) through d)) in the Detailed Description Of the Invention for a detailed description of the rules for defining motivational coping techniques.

REPULSIVE ADVANCED-ORIENTED TECH 26

This component is used by the facilitator to aid the facilitator to define and classify those motivational coping techniques which are “repulsive” or extrinsic oriented motivational coping techniques (e.g., push energy type) and “active-oriented” or advanced-oriented as compared to its associated “passive-oriented” or beginning-oriented motivational coping technique. See FIG. 2.0.0.0.

ATTRACTION ADVANCED-ORIENTED TECH 27

This component is used by the facilitator to aid the facilitator to define and classify those motivational coping techniques which are “attraction” or intrinsic oriented motivational coping techniques (e.g., pull energy type) and “active-oriented” or advanced-oriented as compared to its associated “passive-oriented” or beginning-oriented motivational coping technique. See FIG. 2.0.0.0.

ATTRACTION BEGINNING-ORIENTED TECH 28

This component is used by the facilitator to aid the facilitator to define and classify those motivational coping techniques which are “attraction” or intrinsic oriented motivational coping techniques (e.g., pull energy type) and “passive-oriented” or beginning-oriented as compared to its associated “active-oriented” or advanced-oriented motivational coping technique. See FIG. 2.0.0.0.

REPULSIVE BEGINNING-ORIENTED TECH 29

This component is used by the facilitator to aid the facilitator to define and classify those motivational coping techniques which are “repulsive” or extrinsic oriented motivational coping techniques (e.g., push energy type) and “passive-oriented” or beginning-oriented as compared to its associated “active-oriented” or advanced-oriented motivational coping technique. See FIG. 2.0.0.0.

COMBINED INQUIRY 30

This component combines or constructs a plurality of inquiry components for each state of mind within every fundamental category which can be generated back to the user for a users discussion topic evaluation. The design of every inquiry is based on a plurality of variables identified in the relationship anatomy model and in components 31 through 39 below. This component also builds either the users “more-like” or “less-like” attitudinal trait description for every fundamental category that matches the discussion number in the user's presentation to the discussion number in the description construct table. See the DESCRIPTION INQUIRY COMPONENT 35 below.

ENERGY LEVEL INQUIRY COMPONENT 31

This component provides the beginning of the inquiry where “passive-oriented” inquiries are designed to begin with “What . . . ?” intended to place a users state of mind in using less energy or effort when investigating a discussion topic and “active-oriented” inquiries are designed to begin with “How . . . ?” intended to place a user's state of mind in using more energy or effort when investigating a discussion topic. In addition, this component uses the time (z-axis) energy field coordinate (e.g., present (0), future (1), or time (−1)) wherein present (0) time inquiries include “What do . . . ” or “How do . . . ” intending to place the user's state of mind in the present time. Future (1) time inquiries include “What will . . . ” or “How will . . . ” intending to place the users state of mind in the future time. Past (−1) time inquiries include “What did . . . ” or “How did . . . ” intending to place the user's state of mind in the past time.

MOTIVATIONAL COPING TECH INQUIRY COMPONENT 32

This component builds in the motivational coping technique into the inquiry that was classified by the facilitator for each state of mind classification described in the MOTIVATIONAL COPING TECH CONSTRUCTOR 24 component described above.

PREFIX INQUIRY COMPONENT 33

This component builds a prefix (e.g., “in”) into the inquiry for inquiries that begin with “What . . . ?”. This is necessary in order that the inquiries are grammatically correct.

MOTIVATIONAL ATTRIBUTE INQUIRY COMPONENT 34

This component builds in the motivational attribute into the inquiry that was classified by the facilitator for each state of mind classification and every fundamental category. Refer to Motivational Attribute Inquiry Component in the terms and definitions section and subsection 2 of Section A in the Detailed Description Of The Invention for a detailed description of this component.

DESCRIPTION INQUIRY COMPONENT 35

This component builds in the user's “more-like” and “less-like” attitudinal trait descriptions into each inquiry for every fundamental category in the discussion generator wherein the user's More Like Description and the users corresponding Less Like Description match the discussion number presented in the users presentation to the discussion number listed for every fundamental category in the Table A—Description Construct Table and described in the DISCUSSION NUMBER 10 component within A—DESCRIPTION CONSTRUCT TABLE 1 component.

FIND MORE-LIKE DESCRIPTION INQUIRY COMPONENT 36

This component finds the users “more-like” attitudinal trait descriptions that are built into the inquiry for every fundamental category in the discussion generator wherein the discussion number presented in the users presentation matches the discussion number listed for every fundamental category in the Table A—Description Construct Table.

FIND LESS-LIKE DESCRIPTION INQUIRY COMPONENT 37

This component finds the users “less-like” attitudinal trait descriptions that are built into the inquiry for every fundamental category in the discussion generator wherein the discussion number presented in the users presentation matches the discussion number listed for every fundamental category in the Table A—Description Construct Table.

LESS-LIKE DESCRIPTION INQUIRY COMPONENT 38

This component uses the value in the charge field coordinate (e.g., “more” or “less”) from B—DISCUSSION GENERATOR component that is designed based on the relationship anatomy model wherein attitudinal trait descriptions are classified into either attitudinal trait descriptions that are “more-like” the user and “less-like” the user. For inquiries where the charge field coordinate equals “less”, the “less-like” attitudinal trait description is built into the inquiry for every fundamental category.

MORE-LIKE DESCRIPTION INQUIRY COMPONENT 39

This component uses the value in the charge field coordinate (e.g., “more” or “less”) from B—DISCUSSION GENERATOR component that is designed based on the relationship anatomy model wherein attitudinal trait descriptions are classified into either attitudinal trait descriptions that are “more-like” the user and “less-like” the user. For inquiries where the charge field coordinate equals “more”, the “more-like” attitudinal trait description is built into the inquiry for every fundamental category.

DEFINE NEXT STATE OF MIND 40

This component is used by the facilitator to define the next state of mind in the discussion generator.

DEFINE NEXT FUNDAMENTAL CATEGORY 41

This component is used by the facilitator to select and record the next fundamental category in the discussion generator.

SELECT DISCUSSION TOPIC 42

This component is used by the user to choose a discussion topic during a user's discussion topic evaluation. This is the first step of the C—USER PRESENTATION 3 component.

DISCUSSION TOPIC EVALUATION STATEMENTS 43

This component is used by the user wherein the user answers three discussion topic evaluation statements for a discussion topic during a user's discussion topic evaluation.

STATEMENT 1 RELATED TO X-AXIS (RELATIVE BOND) 44

This component is used by the user wherein the user answers the first of three discussion topic evaluation statements for a discussion topic during a user's discussion topic evaluation. This discussion topic evaluation statement relates to the x-axis (e.g., relative bond) and represents the first digit of a triplet for the energy field coordinates in the discussion generator, wherein the user answers a statement (e.g., “I feel stable when it comes to my <discussion topic>”) “yes” or “no”. Designed into the relationship anatomy model (see FIG. 2.0.0.0), a “yes” answer equals a value of 1 wherein this answer represents a “repulsive” type of motivational coping technique. A “no” answer equals a value of 0 wherein when this answer is selected it represents an “attraction” type of motivational coping techniques. Refer to subsection 2a in Section B in the Detailed Description Of The Invention for more information regarding this component.

RESULT IS “0” 45

This component is used by the user wherein the user answers “no” to the discussion topic statement described above in the STATEMENT 1 RELATED TO X-AXIS (RELATIVE BOND) 44 component.

RESULT IS “1” 46

This component is used by the user wherein the user answers “yes” to the discussion topic statement described above in the STATEMENT 1 RELATED TO X-AXIS (RELATIVE BOND) 44 component.

STATEMENT 2 RELATED TO Y-AXIS (ENERGY) 47

This component is used by the user wherein the user answers the second of three discussion topic evaluation statements for a discussion topic during a user's discussion topic evaluation. This discussion topic evaluation statement relates to the y-axis (e.g., energy) and represents the second digit of a triplet for the energy field coordinates in the discussion generator, wherein the user answers a statement (e.g., “I feel enthusiastic when it comes to my <discussion topic>”) “yes” or “no”. Designed into the relationship anatomy model (see FIG. 2.0.0.0), a “yes” answer equals a value of 1 wherein this answer represents a “higher” energy or more advanced-oriented type of motivational coping technique. A “no” answer equals a value of 0 wherein when this answer is selected it represents a “lower” or more beginning-oriented type of motivational coping techniques. Refer to subsection 2b in Section B in the Detailed Description Of The Invention for more information regarding this component.

RESULT IS “0” 48

This component is used by the user wherein the user answers “no” to the discussion topic statement described above in the STATEMENT 2 RELATED TO Y-AXIS (ENERGY) 47 component.

RESULT IS “0” 49

This component is used by the user wherein the user answers “yes” to the discussion topic statement described above in the STATEMENT 2 RELATED TO Y-AXIS (ENERGY) 47 component.

STATEMENT 3 RELATED TO Z-AXIS (TIME) 50

This component is used by the user wherein the user answers the third of three discussion topic evaluation statements for a discussion topic during a user's discussion topic evaluation. This discussion topic evaluation statement relates to the z-axis (e.g., time) and represents the third digit of a triplet for the energy field coordinates in the discussion generator, wherein the user answers a statement (e.g., “I feel confident when it comes to my <discussion topic>”) “yes” or “no” or “used to be”. Designed into the relationship anatomy model (see FIG. 2.0.0.0), a “no” answer equals a value of 0 wherein this answer represents a present time user reference. A “yes” answer equals a value of 1 wherein this answer represents a future time user reference. A “used to be” answer equals a value of −1 wherein this answer represents a past time user reference. Refer to subsection 2c in Section B in the Detailed Description Of The Invention for more information regarding this component.

RESULT IS “0” 51

This component is used by the user wherein the user answers “no” to the discussion topic statement described above in the STATEMENT 3 RELATED TO Z-AXIS (TIME) 50 component.

RESULT IS “1” 52

This component is used by the user wherein the user answers “yes” to the discussion topic statement described above in the STATEMENT 3 RELATED TO Z-AXIS (TIME) 50 component.

RESULT IS “−1” 53

This component is used by the user wherein the user answers “used to be” to the discussion topic statement described above in the STATEMENT 3 RELATED TO Z-AXIS (TIME) 50 component.

GENERATE DISCUSSION TOPIC INQUIRIES 54

This component uses the triplet constructed from the users answers in the discussion topic evaluation statements described in the components STATEMENT 1 RELATED TO X-AXIS (RELATIVE BOND) 44, STATEMENT 2 RELATED TO Y-AXIS (ENERGY) 47, and in STATEMENT 3 RELATED TO Z-AXIS (TIME) 50 described above (e.g., 011). This triplet uses the field in the discussion generator called the concatenate energy field coordinates and looks up said triplet and finds the associated inquiry for each fundamental category then displays these inquires in the user presentation interface (see FIG. 3.0.0.0).

FIND FIRST FUNDAMENTAL CATEGORY 55

For generating inquiries this component finds the first fundamental category listed in the discussion generator.

PRESENT DISCUSSION TOPIC INQUIRIES 56

For generating discussion topic inquires this component uses the triplet constructed from the users answers in the discussion topic evaluation statements described in the components STATEMENT 1 RELATED TO X-AXIS (RELATIVE BOND) 44, STATEMENT 2 RELATED TO Y-AXIS (ENERGY) 47, and in STATEMENT 3 RELATED TO Z-AXIS (TIME) 50 described above (e.g., 011). This triplet uses the field in the discussion generator called the concatenate energy field coordinates and looks up said triplet and finds the associated inquiry in a field called combined inquiry which is displayed in the user presentation interface (see FIG. 3.0.0.0).

CONCATENATE DISC TOPIC EVALUATION STATEMENT RESULTS 57

For generating discussion topic inquires this component concatenates the triplet constructed from the user's answers in the discussion topic evaluation statements described in the components STATEMENT 1 RELATED TO X-AXIS (RELATIVE BOND) 44, STATEMENT 2 RELATED TO Y-AXIS (ENERGY) 47, and in STATEMENT 3 RELATED TO Z-AXIS (TIME) 50 described above (e.g., 011).

STATEMENT 1 RESULT 58

For generating discussion topic inquiries this component represents the user's answer described in the STATEMENT 1 RELATED TO X-AXIS (RELATIVE BOND) 44 component. This answer represents the first of the triplet (i.e., relative bond x-axis) in the energy field coordinates of the discussion generator.

STATEMENT 2 RESULT 59

For generating discussion topic inquiries this component represents the user's answer described in the STATEMENT 2 RELATED TO Y-AXIS (ENERGY) 47 component. This answer represents the second of the triplet (i.e., energy y-axis) in the energy field coordinates of the discussion generator.

STATEMENT 3 RESULT 60

For generating discussion topic inquiries this component represents the user's answer described in the STATEMENT 3 RELATED TO Z-AXIS (TIME) 50 component. This answer represents the third of the triplet (i.e., time z-axis) in the energy field coordinates of the discussion generator.

LOOK UP COMBINDED INQUIRY 61

For generating discussion topic inquiries this component looks up the combined inquiry listed in the discussion generator for each fundamental category wherein the concatenated energy field coordinates field in the discussion generator equals the triplet developed from the CONCATENATE DISC TOPIC EVALUATION STATEMENT RESULTS 57 component described above. For each fundamental category this inquiry will be displayed in the user presentation for a users discussion topic evaluation.

FIND NEXT FUNDAMENTAL CATEGORY 62

For generating inquiries this component finds the next fundamental category listed in the discussion generator.

### DETAILED DESCRIPTION OF THE METHOD OF THE INVENTION

The following steps are performed:
[1] In column 1 of Table A—Description Construct Table and according to the DEFINE FIRST FUNDAMENTAL CATEGORY 4 component and DEFINE NEXT FUNDAMENTAL CATEORY 17 component in FIG. 1.1.0.0, enter the first and consecutive fundamental categories. Enter the same fundamental category for each description component (e.g., Acceptance). For example, the fundamental category “honor” will be entered 11 times, one for each description component. Other fundamental categories entered the same way by the facilitator are: “devotion”, “confidence”, and “patience”.
[2] In column 3 of Table A—Description Construct Table and according to the MOTIVATIONAL ATTRIBUTE 5 component in FIG. 1.1.0.0, the facilitator enters the motivational attribute that corresponds to its associated fundamental category. Enter the same motivational attribute for each description component (e.g., Acceptance). For example, the fundamental category “honor” has a motivational attribute of “strength”. “Strength” will be entered 11 times, one for each description component. Other motivational attributes entered the same way by the facilitator are “interest” for the fundamental category “devotion”, “need” for the fundamental category “confidence” and “stress” for the fundamental category “patience”. The facilitator confirms that the motivational attributes entered are nouns.
[3] In column 5 of Table A—Description Construct Table and according to the DESCRIPTOIN TYPE 6 component in FIG. 1.1.0.0, the facilitator enters the description type of the fundamental category. For example, the fundamental category of “honor” is “independent” and for additional fundamental categories the facilitator enters “independent” for “devotion”, “independentA” for “confidence”, and “dependentA” for “patience (the letter “A” in the entry ties an “independent” description type to its associated “dependent” description type fundamental category. For additional “independent” and associated “dependent” description types will tie together with the next letter “B”, then “C”, and so on).
[4] In column 6 of Table A—Description Construct Table and according to the DESCRIPTOIN COMPONENT 7 component in FIG. 1.1.0.0, the facilitator enters the same series of description components for each fundamental category. These description components classify each pair of attitudinal trait descriptions. For example, for the fundamental category of “honor” the description components are in called (in order): Acceptance, Activity, Advantage, Authority, Challenge, Change, Empathy, Esteem, Freedom, Structure, and thought. The facilitator enters the same eleven description components in the same order for each fundamental category (e.g., “devotion”, “confidence”, and “patience”)
[5] In column 6 and 7 of Table A—Description Construct Table and according to the DESCRIPTOIN A 8 and DESCRIPTOIN B 9 components in FIG. 1.1.0.0, the facilitator enters for each description component (e.g., Acceptance) the Description A attitudinal trait description (e.g., able to work well alone) and the Description B attitudinal trait description (e.g., friendly and easy to know). The facilitator enters a Description A attitudinal trait description and a Description B attitudinal trait description for each description component and for every fundamental category.
[6] In column 12 of Table A—Description Construct Table and according to the DESCRIPTOIN NUMBER 10 component in FIG. 1.1.0.0, the facilitator enters for each description component (e.g., Acceptance) a series of numbers starting at one (e.g., 1) through eleven (e.g., 11) for each fundamental category. The discussion number is referenced in the Table C—User Presentation for selecting a specific attitudinal trait description for each fundamental category in the Table A—Description Construct Table.
[7] In column 11 of Table A—Description Construct Table and according to the USER DESCRIPTION CHOICE 11 component in FIG. 1.1.1.0, the user enters for each pair of attitudinal trait descriptions and for every fundamental category, Description A shown in the USER CHOOSES DESCRIPTION A 12 component in FIG. 1.1.1.0 and Description B shown in the USER CHOOSES DESCRIPTION B 13 component in FIG. 1.1.1.0, either A or B. The user enters A if the Description A attitudinal trait description is “more-like” themselves or the user enters B if the Description B attitudinal trait description is “more-like” themselves.
[8] In column 8 of Table A—Description Construct Table and according to the MORE LIKE DESCRIPTION 14 component in FIG. 1.1.1.0, for each pair of attitudinal trait descriptions the attitudinal trait description chosen by the user (either Description A or Description B) to be “more-like” themselves is stored. And according to the LESS LIKE DESCRIPTION 15 component in FIG. 1.1.1.0, the attitudinal trait description NOT chosen by the user (either Description A or Description B) is designated to be “less-like” themselves is stored. Also refer to FIG. 2.0.0.0 in substructure 1 called charge fields for a visual representation of how the “more-like” and “less-like” attitudinal trait descriptions relate to the relationship anatomy model (see FIG. 2.1.0.0 through 2.4.0.0).
[9] According to the NEXT DESCRIPTION COMPONENT 16 and the DEFINE NEXT FUNDAMENTAL CATEGORY 17 component in FIG. 1.1.0.0, the facilitator and user repeat steps [1] through [8] steps above for every description component and fundamental category until all fundamental categories are processed.
[10] In column 1 of Table B—Discussion Generator and according to the DEFINE FIRST FUNDAMENTAL CATEGORY 18 component and DEFINE NEXT FUNDAMENTAL CATEORY 41 component in FIG. 1.2.0.0, the facilitator enters the first and consecutive fundamental categories. Enter the same fundamental category for each state of mind (e.g., A through L). For example, the fundamental category “honor” will be entered 12 times, one for each state of mind. Other fundamental categories entered the same way by the facilitator are: “devotion”, “confidence”, and “patience”.
[11] In column 2 of Table B—Discussion Generator and according to the DEFINE FIRST STATE OF MIND 19 component and DEFINE NEXT STATE OF MIND 40 component in FIG. 1.2.0.0, the facilitator enters the first and consecutive state of mind classifications. For example, for each fundamental category (e.g., “honor”) enter A through L which represents each state of mind.
[12] In columns 3, 4, and 5 of Table B—Discussion Generator and according to the ENERGY FIELD COORDINATES (X,Y,Z) 20 component and the RELATIVE BOND (X-AXIS) 21 component and the ENERGY (Y-AXIS) 22 and the TIME (Z-AXIS) 23 in FIG. 1.2.0.0, for every state of mind and for each fundamental category the facilitator enters the triplet (e.g., 100) according to the design of the relationship anatomy model wherein the first of the triplet represents the relative bond x-axis, the second of the triplet represents the energy y-axis, and the third of the triplet represents the time z-axis. Refer to subsection 8b in Section A of the Detailed Description of the Invention for a detailed description of the energy field coordinates and how they relate to the design of the relationship anatomy model.
[13] In columns 6 and 7 of Table B—Discussion Generator the triplet defined in the ENERGY FIELD COORDINATES (X,Y,Z) 20 component from step [12] above are concatenated together (e.g., a triplet, 110) in the field called the concatenate energy field coordinates in column 6 and then converted into a wave function in the field called wave function |ψ> in column 7 (e.g., |100>). Refer to subsection 8 of Section A of the Detailed Description of the Invention for a detailed description of how the wave functions relate to the relationship anatomy model in FIG. 2.0.0.0 through 2.4.0.0.
[14] In column 12 of Table B—Discussion Generator in a field called the motivational coping technique inquiry component and according to the MOTIVATIONAL COPING TECH CONSTRUCTOR 24 component in FIG. 1.2.0.0 and the APPLY MOTIVATIONAL COPING TECH RULES 25 component, the REPULSIVE ADVANCED-ORIENTED TECH 26 component, the ATTRACTION ADVANCED-ORIENTED TECH 27 component, the ATTRACTION BEGINNING-ORIENTED TECH 28 component, and the REPULSIVE BEGINNING-ORIENTED TECH component in FIG. 1.2.1.0, for each fundamental category and according to the wave function descriptions defined in subsection 8b and the rules for defining and the descriptions for each motivational coping technique in subsection 9 of Section A in the Detailed Description of the Invention the facilitator enters the motivational coping techniques.
[15] In column 11 of Table B—Discussion Generator and according to the ENERGY LEVEL INQUIRY COMPONENT 31 component1 in FIG. 1.2.2.0 is the first inquiry component of the COMBINED INQUIRY 30 component in FIG. 1.2.0.0 and FIG. 1.2.2.0. In column 11 the facilitator writes a conditional formula in the excel spreadsheet cell for each state of mind and for every fundamental category other than the dependent fundamental category “patience” wherein if the energy field coordinate energy y-axis coordinate equals 0 (e.g., low energy) then if the energy field coordinate time z-axis coordinate equals 0 (e.g., present time) the facilitator enters “What do you”, else if the time z-axis coordinate equals 1 (e.g., future time) the facilitator enters “What will you”, else if the time z-axis coordinate equals −1 (e.g., past time) the facilitator enters “What did you”. When the energy field coordinate energy y-axis coordinate equals 1 (e.g., high energy) then if the energy field coordinate time z-axis coordinate equals 0 (e.g., present time) the facilitator enters “How do you”, else if the time z-axis coordinate equals 1 (e.g., future time) the facilitator enters “How will you”, else if the time z-axis coordinate equals −1 (e.g., past time) the facilitator enters “How did you”. The conditional formula written in the spreadsheet cell for each state of mind and every fundamental category other than “patience” is as follows: IF(D4=0,IF(E4=0,“What do you”,IF(E4=1,“What will you”,“What did you”)),IF(E4=0,“How do you”,IF(E4=1,“How will you”,“How did you”))). For the dependent fundamental category “patience” and for each state of mind within such fundamental category the facilitator enters the word “*Hint:” which is designed to begin each such energy level inquiry component and then if the energy field coordinate energy y-axis coordinate equals 0 (e.g., low energy) then if the energy field coordinate time z-axis coordinate equals 0 (e.g., present time) the facilitator enters “ ” (i.e., “blank”), else if the time z-axis coordinate equals 1 (e.g., future time) the facilitator enters “ ” (i.e., “blank”), else if the time z-axis coordinate equals −1 (e.g., past time) the facilitator enters “what did you”. When the energy field coordinate energy y-axis coordinate equals 1 (e.g., high energy) then if the energy field coordinate time z-axis coordinate equals 0 (e.g., present time) the facilitator enters “ ” (i.e., “blank”), else if the time z-axis coordinate equals 1 (e.g., future time) the facilitator enters “” (i.e., “blank”), else if the time z-axis coordinate equals −1 (e.g., past time) the facilitator enters “how did you”. The conditional formula written in the spreadsheet cell for each state of mind within the fundamental category “patience” is as follows: “*Hint:” & IF(D40=0,IF(E40=0,″″, IF(E40=1,″″, “what did you”)),IF(E40=0,″″,IF(E40=1,″″,“how did you”))).
[16] In column 12 of Table B—Discussion Generator in a field called the motivational coping technique inquiry component described in step [14] above and according to the MOTIVATIONAL COPING TECH INQUIRY COMPONENT 32 component in FIG. 1.2.2.0 is the second inquiry component of the COMBINED INQUIRY 30 component in FIG. 1.2.0.0 and FIG. 1.2.2.0.
[17] In column 13 of Table B—Discussion Generator in a field called the prefix inquiry component and according to the PREFIX INQUIRY COMPONENT 33 component in FIG. 1.2.2.0 is the third inquiry component of the COMBINED INQUIRY 30 component in FIG. 1.2.0.0 and FIG. 1.2.2.0 which is defined for each state of mind and for every fundamental category other than the dependent fundamental category “patience” wherein the facilitator enters a conditional formula in each cell that if the energy field coordinate energy y-axis value equals 0 (e.g., low energy) then the facilitator enters the word “in”, otherwise the prefix inquiry component is left blank. The formula is as follows: IF(D4=0,“in”,“ ”). For the dependent fundamental category “patience” and defined for each state of mind within the fundamental category “patience”, the facilitator enters a conditional formula in each cell that if the energy field coordinate energy y-axis value equals 0 (e.g., low energy) then the facilitator enters “ ” (e.g., blank), otherwise if the energy field coordinate time z-axis value equals −1 then the facilitator enters the word “in”, otherwise the facilitator enters “ ” (e.g., blank). The formula is as follows: IF(D40=0,IF(E40=−1,“in”.“ ”),“ ”).
[18] In column 8 of Table B—Discussion Generator in the field called the charge field coordinate is representative of the relationship anatomy model according to FIG. 2.0.0.0 through 2.4.0.0 wherein for each state of mind and for every fundamental category (e.g., independent and dependent description types) the “more-like” and “less-like” attitudinal trait descriptions coordinate is represented. According to subsection 8a in Section A the quadrant caches 1 through 4 follow a distinct order, at least in one embodiment of the invention, wherein the quadrant 1 cache represents attitudinal trait descriptions “more-like” the user and the quadrant 2 cache represents attitudinal trait descriptions “less-like” the user. Quadrant 3 cache represents attitudinal trait descriptions “more-like” the user and the quadrant 4 cache represents attitudinal trait descriptions “less-like” the user. As represented here, the charge field coordinate starts with “more”, then “less”, then “more”, then “less” following the pattern of the quadrant cache order for every relationship anatomy model module in each of the three time (z-axis) measures (e.g., present, future, and past; see FIG. 2.1.0.0 through 2.4.0.0). Therefore, the facilitator enters for each state of mind beginning with state of mind “A” which represents the quadrant 1 cache in the relationship anatomy model in FIG. 2.0.0.0 through 2.4.0.0, the word “more” and then for state of mind “B” which represents the quadrant 2 cache the facilitator enters the word “less” and then for state of mind “C” which represents the quadrant 3 cache the facilitator enters the word “more” and then for state of mind “D” which represents the quadrant 4 cache the facilitator enters the word “less” and so on until each state of mind in each time (z-axis) measure and for every fundamental category is represented with a charge field coordinate of either “more” or “less”. This field is used as a portion of combining the inquiry represented in the COMBINED INQUIRY 30 component in FIG. 1.2.0.0 and FIG. 1.2.2.0 and more specifically the value in this field, charge field coordinate (e.g., “more” or “less”) is represented in the DESCRIPTION INQUIRY COMPONENT 35 in FIG. 1.2.2.0 and in the FIND MORE-LIKE DESCRIPTION INQUIRY component and the FIND LESS-LIKE DESCRIPTION INQUIRY component in FIG. 1.2.2.1.
[19] In column 14 of Table B—Discussion Generator in a field called the motivational attribute inquiry component and according to the MOTIVATIONAL ATTRIBUTE INQUIRY COMPONENT 34 in FIG. 1.2.2.0 is the fourth inquiry component of the COMBINED INQUIRY 30 component in FIG. 1.2.0.0 and FIG. 1.2.2.0 which is defined for each state of mind and for every fundamental category. For each change in the motivational attribute (e.g., “strength”, “interest”, “need”, and “stress”) which equates to each change in fundamental category, the facilitator enters a phrase that both utilizes the motivational attribute and incorporates supporting whether a user's “more-like” or “less-like” attitudinal trait description will be constructed into the combined inquiry. For example, the facilitator enters a formula into this field and changes it for each motivational attribute to represent the optimal way in which to phrase such a motivational attribute inquiry component. Refer to the Table B—Discussion Generator and locate the first state of mind “A” for the fundamental category “honor”. Then locate column 14 wherein for this state of mind “A” and the fundamental category “honor”, the facilitator enters a phrase using a formula wherein the formula first identifies the charge field coordinate of either “more” or “less”. If the charge field coordinate equals “more” (as in the case in state of mind “A”) then the facilitator enters the phrase “your strength of being” wherein the word “your” represents attitudinal trait descriptions “more-like” the user. If the charge field coordinate equals “less” (as it does in the state of mind “B” for the fundamental category “honor”) then the facilitator enters the phrase “others strength of being” wherein the word “others” represent attitudinal trait descriptions “less-like” the user. The conditional formula entered by the facilitator for each state of mind (e.g., A through L) in the fundamental category “honor” is as follows (note that H represents the charge field coordinate): IF(H4=“more”,“your strength of being”,“others strength of being”) wherein “strength” is the motivational attribute for the fundamental category “honor”. In order for the facilitator to optimize the structural integrity of each inquiry the phrases change slightly for each motivational attribute. So, for every entry of the motivational attribute inquiry component field for every state of mind and for each fundamental category the conditional formula entered by the facilitator for each state of mind (e.g., A through L) in the fundamental category “devotion” is as follows (note that H represents the charge field coordinate): IF(H16=“more”,“your interest in”,“others interest in”) wherein “interest” is the motivational attribute for the fundamental category “devotion”. Next, the conditional formula entered by the facilitator for each state of mind (e.g., A through L) in the fundamental category “confidence” is as follows (note that H represents the charge field coordinate): IF(H28=“more”,“your need for”,“others need for”) wherein “need” is the motivational attribute for the fundamental category “confidence”. Last, the conditional formula entered by the facilitator for each state of mind (e.g., A through L) in the fundamental category “patience” is as follows (note that H represents the charge field coordinate): IF(H40=“more”,“your stress toward”,“others stress toward”) wherein “stress” is the motivational attribute for the fundamental category “patience”.
[20] In column 10 of Table A—Description Construct Table in the field called the doublekey which represents a concatenation of the fundamental category in column 1 according to the DEFINE FIRST FUNDAMENTAL CATEGORY 4 component and the DEFINE NEXT FUNDAMENTAL CATEGORY 17 component in FIG. 1.1.0.0, and the discussion number in column 12 according to the DISCUSSION NUMBER 10 component in FIG. 1.1.0.0. The facilitator enters a formula in column 10 for every fundamental category as follows: CONCATENATE(A3,M3). For example, the doublekey entered for the fundamental category “honor” wherein the discussion number is 11 is “honor11”. The doublekey is necessary for completing the next step.

[21] In column 9 of Table B—Discussion Generator in the field called the fundamental category description choice represents the description choice chosen by the user (e.g., either A or B) according to the USER DESCRIPTION CHOICE 11 component described in steps [7] and [8] wherein for each fundamental category the discussion number in Table A—Description Construct Table matches the discussion number (e.g., 1 through 11) displayed on the Table C—User Presentation during a user's discussion topic evaluation. For example, if the discussion number displayed on the Table C—User Presentation equals 11 (see FIG. 3.0.0.0) then the user's choice A or B is identified for the fundamental category “honor” wherein the discussion number on the Table A—Description Construct Table equals 11. For the discussion number 11 in the fundamental category “honor” in the Table A—Description Construct Table the user chose “B”. Therefore, “B” is displayed in the Table B—Discussion Generator for every state of mind for the fundamental category “honor” (see column 9 in the Table B—Discussion Generator). In order to identify each of the users choices, the facilitator enters in column 9 of Table B—Discussion Generator a look up formula for every state of mind and in each fundamental category. The look up formula first concatenates the fundamental category in column 1 (e.g., “honor”) and the discussion number displayed on the Table C—User Presentation (e.g., 11). The concatenation of these data elements replicates the doublekey field described in step 20 above. The concatenation of these data elements (e.g., “honor11”) is used to look up the Description Choice Evaluation Procedure entry (e.g., B) in the Table A—Description Construct Table and displays the Description Choice Evaluation Procedure (e.g., B) in the fundamental category description choice field in column 9 of the Table B—Discussion Generator. The formula entered by the facilitator in column 9 is as follows: VLOOKUP(CONCATENATE(A4,″ ‘C—User Presentation’!$F$13),‘A—Description Construct Table’!K:L,2,FALSE).
[22] In column 2 of Table A—Description Construct Table in the field called the tripletkey which represents a concatenation of the fundamental category in column 1 according to the DEFINE FIRST FUNDAMENTAL CATEGORY 4 component and the DEFINE NEXT FUNDAMENTAL CATEGORY 17 component in FIG. 1.1.0.0, the discussion number in column 12 according to the DISCUSSION NUMBER 10 component in FIG. 1.1.0.0, and the description choice evaluation procedure in column 11 according to the USER DESCRIPTION CHOICE 11 component in FIG. 1.1.0.0 and in FIG. 1.1.1.0. The facilitator enters a formula in column 2 for every fundamental category as follows: CONCATENATE(A3,M3,L3). For example, the tripletkey entered for the fundamental category “honor” wherein the discussion number is 11 and the description choice evaluation procedure is “B” is “honor11B”. The tripletkey is necessary for completing the following step.
[23] In columns 15 and 16 of Table B—Discussion Generator in the fields called the more like description inquiry component (e.g., column 15) and less like description inquiry component (e.g., column 16) according to the DESCRIPTION INQUIRY COMPONENT 35 component in FIG. 1.2.2.0 and in FIG. 1.2.2.1 is the fifth and final inquiry component of the COMBINED INQUIRY 30 component in FIG. 1.2.0.0 and FIG. 1.2.2.0 which is defined for each state of mind and for every fundamental category wherein either the “more-like” user's description according to the FIND MORE-LIKE DESCRIPTION INQUIRY 37 sub-component or the “less-like” users description according to the FIND LESS-LIKE DESCRIPTION INQUIRY COMPONENT 38 sub-component will be selected as the fifth and final inquiry component of the COMBINED INQUIRY 30 component. The more like description inquiry component (e.g., column 15) is identified by a formula entered in column 15 by the facilitator wherein the formula first concatenates the fundamental category (e.g., honor), the discussion number on the users presentation (see FIG. 3.0.0.0) (e.g., 11), and the fundamental category description choice in column 9 (e.g., B). The concatenation of these data elements replicates the tripletkey described in step 22 above. The concatenation of these data elements (e.g., “honor11B”) is used as a primary key to look up the More Like Description (e.g., attitudinal trait description) in the Table A—Description Construct Table and displays the “more-like” user description in the more like description inquiry component field in column 15 of the Table B—Discussion Generator. The formula that the facilitator enters for the more like description inquiry component in column 15 is as follows: VLOOKUP(CONCATENATE(A4,‘C—User Presentation’!$F$13,|3),‘A—Description Construct Table’!B: J,8,FALSE). The less like description inquiry component (e.g., column 16) is identified by a formula entered in column 15 by the facilitator wherein the formula first concatenates the fundamental category (e.g., honor), the discussion number on the users presentation (see FIG. 3.0.0.0) (e.g., 11), and the fundamental category description choice in column 9 (e.g., B). The concatenation of these data elements replicates the tripletkey described in step 22 above. The concatenation of these data elements (e.g., “honor11B”) is used as a primary key to look up the Less Like Description (e.g., attitudinal trait description) in the Table A—Description Construct Table and displays the “less-like” user description in the less like description inquiry component field in column 16 of the Table B—Discussion Generator. The formula that the facilitator enters for the less like description inquiry component in column 16 is as follows: VLOOKUP(CONCATENATE(A3,‘C—User Presentation’!$F$13,|3),‘A—Description Construct Table’!B:J,9,FALSE).
[24] In column 10 of Table B—Discussion Generator in the field called the combined inquiry according to the COMBINED INQUIRY 30 component in FIG. 1.2.0.0 and in FIG. 1.2.2.0 is the concatenation of all five inquiry components described in steps [15] (e.g., column11), [16] (e.g., column12), [17] (e.g., column13),[19] (e.g., column14), and [23] (e.g., either column15 or column 16) above. In column 10 for every state of mind and for each fundamental category, the facilitator enters a formula which concatenates the energy level inquiry component in column 11, the motivational coping technique inquiry component in column 12, the prefix inquiry component in column 13, the motivational attribute inquiry component in column 14, and either the more like description component in column 15 or the less like description component in column 16. The formula that the facilitator enters in column 10 is designed according to the LESS LKE DESCRIPTION INQUIRY COMPONENT 38 component and the MORE LIKE DESCRIPTION INQUIRY COMPONENT 39 component in FIG. 1.2.2.0. The formula is designed to choose the more like description component in column 15 when the charge field coordinate (as described in step [18] above and in subsection 4 of Section A) equals “more” and it is designed to choose the less like description component in column 16 when the charge field coordinate equals “less”. The formula that the facilitator enters is as follows: CONCATENATE(K4,” “,L4,” “,N4,” “,O4,” “,IF(H4=“more”,P4,Q4),“?”. The result as shown for state of mind “G” in the fundamental category “honor” is—How will you observe your strength of being able to handle ambiguous situations well? (see FIG. 3.0.0.0).
[25] The first of three steps in the users discussion topic evaluation are presented in the Table C—User Presentation and in FIG. 3.0.0.0. According to the SELECT DISCUSSION TOPIC 42 component in FIG. 1.3.0.0 represents the first step in the users discussion topic evaluation. In the first step of the user discussion topic evaluation the user selects a discussion topic. The facilitator pre-defines a set of choices wherein the user selects a pull down menu on the user presentation. The choices from which the user may choose are: family, friends, finances, work, and home. For example, the user may choose “work”. Refer to subsection 1 of Section B in the Detailed Description of the Invention.
[26] The second of three steps in the users discussion topic evaluation are presented in the Table C—User Presentation and in FIG. 3.0.0.0. According to the DESCRIPTION TOPIC EVALUATION STATEMENTS 43 component in FIG. 1.3.0.0 represents the second step in the users discussion topic evaluation. In the second step of the user discussion topic evaluation the user answers three discussion topic evaluation statements (e.g., a), b), and c)) regarding the discussion topic (e.g., “work). The answers given by the user for these three statements build the triplet (e.g., 011) which represents the three energy field coordinates (relative bond x-axis, energy y-axis, and time z-axis) wherein the first discussion topic evaluation statement (e.g., a)) relates directly to the relative bond x-axis, the second discussion topic evaluation statement (e.g., b)) relates directly to the energy y-axis, and the third discussion topic evaluation statement (e.g., c)) relates directly to the time y-axis. For the first of the three discussion topic evaluation statements according to STATEMENT 1 RELATED TO X-AXIS (RELATIVE BOND) 44 component in FIG. 1.3.0.0 the facilitator enters a) I feel stable when it comes to my <discussion topic> (e.g., work). This statement is designed to obtain the first digit of the triplet which relates directly with the relative bond x-axis in the relationship anatomy model. This statement requires the user to answer one of two ways, either “no” or “yes” wherein “no” equals 0 according to the RESULT IS “0” 45 component in FIG. 1.3.0.0 and “yes” equals 1 according to the RESULT IS “1” 46 component in FIG. 1.3.0.0 (e.g., see in FIG. 3.0.0.0 that the user answers “no”; equals 0). For the second of the three discussion topic evaluation statements according to STATEMENT 2 RELATED TO Y-AXIS (ENERGY) 47 component in FIG. 1.3.0.0 the facilitator enters a) I feel enthusiastic when it comes to my <discussion topic> (e.g., work). This statement is designed to obtain the second digit of the triplet which relates directly with the energy x-axis in the relationship anatomy model. This statement requires the user to answer one of two ways, either “no” or “yes” wherein “no” equals 0 according to the RESULT IS “0” 48 component in FIG. 1.3.0.0 and “yes” equals 1 according to the RESULT IS “1” 49 component in FIG. 1.3.0.0 (e.g., see in FIG. 3.0.0.0 that the user answers “yes”; equals 1). For the third of the three discussion topic evaluation statements according to STATEMENT 3 RELATED TO Z-AXIS (TIME) 50 component in FIG. 1.3.0.0 the facilitator enters a) I feel confident when it comes to my <discussion topic> (e.g., work). This statement is designed to obtain the third digit of the triplet which relates directly with the time y-axis in the relationship anatomy model. This statement requires the user to answer one of three ways, either “no” or “yes” or “used to be” wherein “no” equals 0 according to the RESULT IS “0” 51 component in FIG. 1.3.0.0 and “yes” equals 1 according to the RESULT IS “1” 52 component in FIG. 1.3.0.0 and “used to be” equals −1 according to the RESULT IS “−1” 53 component in FIG. 1.3.0.0 (e.g., see in FIG. 3.0.0.0 where the user answers “yes”; equals 1). For a detailed description of how the discussion topic evaluation statements build the triplet (e.g., 011) and relate to the energy field coordinates in the Table B—Discussion Generator and in the relationship anatomy model in FIG. 2.0.0.0 through 2.4.0.0 refer to subsection 2a through 2c in Section B in the Detailed Description of the Invention.
[27] According to the GENERATE DISCUSSION TOPIC INQUIRIES 54 component in FIG. 1.3.0.0 and FIG. 1.3.1.0 the system utilizes the answers given (e.g., the triplet; 011) by the user in step [26] above and for each fundamental category generates the most optimum inquiries for the user to use to investigate their discussion topic from the discussion topic evaluation. In FIG. 3.0.0.0, under step 3) Discussion Topic Inquiries, there are four inquiries displayed, one for each fundamental category. Inquiry a) relates to the fundamental category “honor”, inquiry b) relates to the fundamental category “devotion”, inquiry c) relates to the fundamental category “confidence”, and inquiry d) relates to the fundamental category “patience” wherein inquiry d) is dependent on inquiry c). For a detailed description of this “dependency” refer to subsection 3 in Section A in the Detailed Description of the Invention. For each fundamental category according to the FIND FIRST FUNDAMENTAL CATEGORY 55 component in FIG. 1.3.1.0 and the FIND NEXT FUNDAMENTAL CATEGORY 62 component in FIG. 1.3.1.0, each fundamental category has an associated inquiry wherein each inquiry presented according to the PRESENT DISCUSSION TOPIC INQUIRY 56 component is displayed in the Table C—User Presentation or for a specific example shown herein in FIG. 3.0.0.0. For every inquiry, the triplet (e.g., 011) concatenated from the user's answers of the discussion topic evaluation statements, for the purpose of showing how these inquiries are generated, are located in FIG. 3.0.0.0 to the right of the discussion number. For each fundamental category and according to the PRESENT DISCUSSION TOPIC INQUIRY 56 in FIG. 1.3.1.0, each of the discussion topic inquiries in step 3 of FIG. 3.0.0.0 (e.g., a) through d)) utilizes the triplet (e.g., 011) according to the CONCATENATE DISC TOPIC EVALUATION STATEMENT RESULTS 57 component in FIG. 1.3.1.0 and according to the STATEMENT 1 RESULT 58 component in FIG. 1.3.1.0 and according to the STATEMENT 2 RESULT 59 component in FIG. 1.3.1.0 and according to the according to the STATEMENT 3 RESULT 60 component in FIG. 1.3.1.0 to look up the inquiry in the Table B—Discussion Generator wherein the triplet (e.g., 011) finds the match for each fundamental category according to the LOOK UP COMBINED INQUIRY 61 component in FIG. 1.3.1.0 in the concatenate energy field coordinates field in the Table B—Discussion Generator and retrieves the inquiry in the inquiry component in the Table B—Discussion Generator and presents the inquiry in the Table C—User Presentation for the user to utilize in their investigation of the discussion topic. In order to do this, the facilitator enters a formula that correlates to each fundamental category in step 3) Discussion Topic Inquiries a) through d) in FIG. 3.0.0.0 as follows for the fundamental category “honor” in step a): “a)” & “ ” & VLOOKUP (CONCATENATE ($G$8,$G$9,$G$10),‘B—Discussion Generator’$F$4:$Q$15,5,FALSE). For the fundamental category “devotion” in step b) the formula is as follows: “b)” & “ ” & VLOOKUP (CONCATENATE ($G$8,$G$9,$G$10),‘B—Discussion Generator’!$F$16:$Q$27,5,FALSE). For the fundamental category “confidence” in step c) the formula is as follows: “c)” & “ ” & VLOOKUP (CONCATENATE ($G$8,$G$9,$G$10),‘B—Discussion Generator’!$F$28:$Q$39,5,FALSE). For the fundamental category “patience” in step d) the formula is as follows: “d)” & “ ” & VLOOKUP (CONCATENATE ($G$8,$G$9,$G$10),‘B—Discussion Generator’$F$40:$Q$51,5,FALSE). The Discussion Topic Statements presented in the Table C—User Presentation or in the example provided in FIG. 3.0.0.0 that are generated from the Table B—Discussion Generator and used by the user to investigate their discussion topic “work” are as follows:
   a) How will you respect your strength of being able to handle ambiguous situations well?
   b) How will you discover your interest in using imagination and intuitiveness?
   c) How will you accept your need for plenty of time to make complex decisions?
   d) *Hint: forgive your stress toward having indecision when pressured?
   *if c) above is not satisfied then d) is more likely to become active.

### Descriptions Of Other Embodiments

Other user profile's which use a binary scale to measure a plurality of different behaviors, attitudes, preferences, etc. within one or more attributes may also be designed to interface with this invention. In addition, combining the results of two or more profiles may also interface with this invention.

In additional embodiments, processes could be designed to utilize the user's feedback (e.g., journal entries) or other relevant data to enhance or evolve and existing user profile wherein the user's experience may be captured and re-generated. Moreover, task management tools may be developed into this invention or an interface may be designed to work with other, well known, task management products.

Also, this design allows for an infinite number of variables (e.g., state of mind) to be defined for each quadrant cache, therefore, video clips may also be generated which provide user's a visual experience of a particular motivational coping technique associated with a particular quadrant cache. For example, if a user is asked to “support” (e.g., a motivational coping technique for the fundamental category “confidence”) another person's “need” (e.g., plenty of time for complex decisions) then a video could be generated that plays a presentation that shows someone “supporting” another person who has a need for “plenty of time for complex decisions”.

Last but not least, this product may also be designed to work over the internet and in conjunction with other user's utilizing this same interface, so that, people may interface together to strengthen their working or personal relationships.

The foregoing discussion of the invention has been presented for purposes of illustration and description. Further, the description is not intended to limit the invention to the form disclosed herein. Consequently, variation and modification commiserate with the above teachings, within the skill and knowledge of the relevant art, are within the scope of the present invention. The embodiment described hereinabove is further intended to explain the best mode presently known of practicing the invention and to enable others skilled in the art to utilize the invention as such, or in other embodiments, and with the various modifications required by their particular application or uses of the invention.

TABLE A
Discussion Construct Table

| column1 | | | column3 | | column5 |
|---|---|---|---|---|---|
| Fundamental | column2 | column3 | Motivational | column4 | Description |
| Category | TRIPLETKEY | BirkmanBehavior | Attribute | Description Type | Component |
| honor | honor1B | Usually | strength | independent | Acceptance |
| honor | honor2A | Usually | strength | independent | Activity |
| honor | honor3A | Usually | strength | independent | Advantage |
| honor | honor4B | Usually | strength | independent | Authority |
| honor | honor5B | Usually | strength | independent | Challenge |
| honor | honor6B | Usually | strength | independent | Change |
| honor | honor7B | Usually | strength | independent | Empathy |
| honor | honor8A | Usually | strength | independent | Esteem |
| honor | honor9B | Usually | strength | independent | Freedom |
| honor | honor10B | Usually | strength | independent | Structure |
| honor | honor11B | Usually | strength | independent | Thought |
| devotion | devotion1A | Organize | interest | independent | Acceptance |
| devotion | devotion2A | Organize | interest | independent | Activity |
| devotion | devotion3A | Organize | interest | independent | Advantage |
| devotion | devotion4B | Organize | interest | independent | Authority |
| devotion | devotion5A | Organize | interest | independent | Challenge |
| devotion | devotion6B | Organize | interest | independent | Change |
| devotion | devotion7A | Organize | interest | independent | Empathy |
| devotion | devotion8B | Organize | interest | independent | Esteem |
| devotion | devotion9B | Organize | interest | independent | Freedom |
| devotion | devotion10A | Organize | interest | independent | Structure |
| devotion | devotion11A | Organize | interest | independent | Thought |
| confidence | confidence1A | Need | need | independentA | Acceptance |
| confidence | confidence2B | Need | need | independentA | Activity |
| confidence | confidence3A | Need | need | independentA | Advantage |
| confidence | confidence4B | Need | need | independentA | Authority |
| confidence | confidence5B | Need | need | independentA | Challenge |
| confidence | confidence6A | Need | need | independentA | Change |
| confidence | confidence7A | Need | need | independentA | Empathy |
| confidence | confidence8A | Need | need | independentA | Esteem |
| confidence | confidence9A | Need | need | independentA | Freedom |
| confidence | confidence10A | Need | need | independentA | Structure |
| confidence | confidence11B | Need | need | independentA | Thought |
| patience | patience1A | Avoid | stress | dependentA | Acceptance |
| patience | patience2B | Avoid | stress | dependentA | Activity |
| patience | patience3A | Avoid | stress | dependentA | Advantage |
| patience | patience4B | Avoid | stress | dependentA | Authority |
| patience | patience5B | Avoid | stress | dependentA | Challenge |
| patience | patience6A | Avoid | stress | dependentA | Change |
| patience | patience7A | Avoid | stress | dependentA | Empathy |
| patience | patience8A | Avoid | stress | dependentA | Esteem |
| patience | patience9A | Avoid | stress | dependentA | Freedom |
| patience | patience10A | Avoid | stress | dependentA | Structure |
| patience | patience11B | Avoid | stress | dependentA | Thought |

| column6 | column7 |
|---|---|
| Description A | Description B |
| able to work well alone | friendly and easy to know |
| like to reflect before acting | takes direct (immediate) action to get things done |
| oriented towards general benefit | oriented toward individual advantage |
| low-key in the exercise of authority | directive and commanding |
| self-confident, focused on success | has high expectations of self and others |
| concentrates attention well | likes a variety of simultaneous tasks |
| objective and detached | sympathetic and warm |
| direct and straightforward | insightful and intuitive |
| understands how most people think | individualistic in outlook |
| flexible and open to new approaches | organized and sequential |
| sees issues in terms of black or white | able to handle ambiguous situations well |
| comfort with one-on-one interaction | positive relationships and mutual trust |
| comfort in problem solving and crisis intervention | ?comfort in managing workloads? |
| ?rewarding others motivation? | hard work - rewarding self motivation |
| a commitment to major responsibilities | ?a commitment to specializing in a process? |
| work that allows stimulating involvement with others | ?work independently? |
| completing any details | approaching issues holistically |
| find comfort with non-traditional work | socially traditional work and people interactions |
| exercising strong managerial authority | a minimum of directive involvement |
| ?learning from trial and error? | educational approaches to growth and development |
| a lead in development processes in specialized areas | a lead in organizational development |
| using imagination and intuitiveness | approaching problems factually and logically |
| plenty of time alone or in small groups | to feel part of the group |
| personal control over scheduling | a busy schedule |
| an environment based on trust | a way to measure personal performance |
| a non-directive, democratic environment | to know who is in charge |
| a success-oriented environment | personal challenges |
| adequate notice of any change | plenty of different calls on attention |
| an unemotional environment | an outlet for subjective issues |
| others to be frank and forthright | respect of key individuals |
| a predictable environment | opportunities for individuality |
| only an outline to follow | a definite plan in place |
| reducing issues to their simplest form | plenty of time to make complex decisions |
| being impatient with group interaction | over valuing group opinion |
| putting things off | failing to delegate when necessary |
| becoming too idealistic | focusing too much on personal payoff |
| failing to address issues of control | becoming domineering and controlling |
| denying responsibility for errors | expecting too much of self and others |
| failing to accept necessary change | getting distracted too easily |
| discounting peoples feelings | worrying unnecessarily |
| being too blunt | feeling unappreciated on occasions |
| discomfort with unusual ideas | being different for its own sake |
| weakness in follow-through | over-insistence on following procedures |
| being impulsive | having indecision when pressured |
| column8 | column9 |
| More Like Description | Less Like Description |
| friendly and easy to know | able to work well alone |
| likes to reflect before acting | takes direct (immediate) action to get things done |
| oriented towards general benefit | oriented toward individual advantage |
| directive and commanding | low-key in the exercise of authority |
| has high expectations of self and others | self-confident focused on success |
| likes a variety of simultaneous tasks | concentrates attention well |
| sympathetic and warm | objective and detached |
| direct and straightforward | insightful and intuitive |
| individualistic in outlook | understands how most people think |
| organized and sequential | flexible and open to new approaches |
| able to handle ambiguous situations well | sees issues in terms of black and white |
| comfort with one-on-one interaction | positive relationships and mutual trust |
| comfort in problem solving and crisis intervention | ?comfort in managing workloads? |
| ?rewarding others motivation? | hard work - rewarding self motivation |
| ?a commitment to specializing in a process? | a commitment to major responsibilities |
| work that allows stimulating involvement with others | ?work independently? |
| approaching issues holistically | completing any details |
| find comfort with non-traditional work | socially traditional work and people interactions |
| a minimum of directive involvement | exercising strong managerial authority |
| educational approaches to growth and development | ?learning from trial and error? |
| a lead in development processes in specialized areas | a lead in organizational development |
| using imagination and intuitiveness | approaching problems factually and logically |
| plenty of time alone or in small groups | to feel part of the group |
| a busy schedule | personal control over scheduling |
| an environment based on trust | a way to measure personal performance |
| to know who is in charge | a non-directive, democratic environment |
| personal challenges | a success-oriented environment |
| adequate notice of any change | plenty of different calls on attention |
| an unemotional environment | an outlet for subjective issues |
| others to be frank ano forthright | respect of key individuals |
| a predictable environment | opportunities for individuality |
| only an outline to follow | a definite plan in place |
| plenty of time to make complex decisions | reducing issues to their simplest form |
| being impatient with group interaction | over valuing group opinion |
| failing to delegate when necessary | putting things off |
| becoming too idealistic | focusing too much on personal payoff |
| becoming domineering and controlling | failing to address issues of control |
| expecting too much of self and others | denying responsibility for errors |
| failing to accept necessary change | getting distracted to easily |
| discounting peoples feelings | worrying unnecessarily |
| being too blunt | feeling unappreciated on occasions |
| discomfort with unusual ideas | being different for its own sake |
| weakness in follow-through | over-insistence on following procedures |
| having indecision when pressured | being impulsive |

| | column11 | |
|---|---|---|
| | Description | |
| | Choice | column12 |
| column10 | Evaluation | Discussion |
| DOUBLEKEY | Procedure | Number |
| honor1 | B | 1 |
| honor2 | A | 2 |
| honor3 | A | 3 |
| honor4 | B | 4 |
| honor5 | B | 5 |
| honor6 | B | 6 |
| honor7 | B | 7 |
| honor8 | A | 8 |
| honor9 | B | 9 |
| honor10 | B | 10 |
| honor11 | B | 11 |
| devotion1 | A | 1 |
| devotion2 | A | 2 |
| devotion3 | A | 3 |
| devotion4 | B | 4 |
| devotion5 | A | 5 |
| devotion6 | B | 6 |
| devotion7 | A | 7 |
| devotion8 | B | 8 |
| devotion9 | B | 9 |
| devotion10 | A | 10 |
| devotion11 | A | 11 |
| confidence1 | A | 1 |
| confidence2 | B | 2 |
| confidence3 | A | 3 |
| confidence4 | B | 4 |
| confidence5 | B | 5 |
| confidence6 | A | 6 |
| confidence7 | A | 7 |
| confidence8 | A | 8 |
| confidence9 | A | 9 |
| confidence10 | A | 10 |
| confidence11 | B | 11 |
| patience1 | A | 1 |
| patience2 | B | 2 |
| patience3 | A | 3 |
| patience4 | B | 4 |
| patience5 | B | 5 |
| patience6 | A | 6 |
| patience7 | A | 7 |
| patience8 | A | 8 |
| patience9 | A | 9 |
| patience10 | A | 10 |
| patience11 | B | 11 |

TABLE B
Discussion Generator

| | | column3 | column4 | column5 | | |
|---|---|---|---|---|---|---|
| | column2 | energy field coordinates | | | column6 | column7 |
| column1 | state of | Relative Bond | EnergyΔ | time | concatenate energy | wave function |
| fundamental cat. | mind | (x-axis) | (y-axis) | (z-axis) | field coordinates | \|ψ> |
| honor | A | 1 | 0 | 0 | 100 | \|100> |
| honor | B | 0 | 0 | 0 | 000 | \|000> |
| honor | C | 1 | 1 | 0 | 110 | \|110> |
| honor | D | 0 | 1 | 0 | 010 | \|010> |
| honor | E | 0 | 0 | 1 | 001 | \|001> |
| honor | F | 1 | 0 | 1 | 101 | \|101> |
| honor | G | 0 | 1 | 1 | 011 | \|011> |
| honor | H | 1 | 1 | 1 | 111 | \|111> |
| honor | I | 1 | 0 | −1 | 10-1 | −\|101> |
| honor | J | 0 | 0 | −1 | 00-1 | −\|001> |
| honor | K | 1 | 1 | −1 | 11-1 | −\|111> |
| honor | L | 0 | 1 | −1 | 01-1 | −\|011> |
| devotion | A | 1 | 0 | 0 | 100 | \|100> |
| devotion | B | 0 | 0 | 0 | 000 | \|000> |
| devotion | C | 1 | 1 | 0 | 110 | \|110> |
| devotion | D | 0 | 1 | 0 | 010 | \|010> |
| devotion | E | 0 | 0 | 1 | 001 | \|001> |
| devotion | F | 1 | 0 | 1 | 101 | \|101> |
| devotion | G | 0 | 1 | 1 | 011 | \|011> |
| devotion | H | 1 | 1 | 1 | 111 | \|111> |
| devotion | I | 1 | 0 | −1 | 10-1 | −\|101> |
| devotion | J | 0 | 0 | −1 | 00-1 | −\|001> |
| devotion | K | 1 | 1 | −1 | 11-1 | −\|111> |
| devotion | L | 0 | 1 | −1 | 01-1 | −\|011> |
| Confidence | A | 1 | 0 | 0 | 100 | \|100> |
| Confidence | B | 0 | 0 | 0 | 000 | \|000> |
| Confidence | C | 1 | 1 | 0 | 110 | \|110> |
| Confidence | D | 0 | 1 | 0 | 010 | \|010> |
| Confidence | E | 0 | 0 | 1 | 001 | \|001> |
| Confidence | F | 1 | 0 | 1 | 101 | \|101> |
| Confidence | G | 0 | 1 | 1 | 011 | \|011> |
| Confidence | H | 1 | 1 | 1 | 111 | \|111> |
| Confidence | I | 1 | 0 | −1 | 10-1 | −\|101> |
| Confidence | J | 0 | 0 | −1 | 00-1 | −\|001> |
| Confidence | K | 1 | 1 | −1 | 11-1 | −\|111> |
| Confidence | L | 0 | 1 | −1 | 01-1 | −\|011> |
| Patience | A | 1 | 0 | 0 | 100 | \|100> |
| Patience | B | 0 | 0 | 0 | 000 | \|000> |
| Patience | C | 1 | 1 | 0 | 110 | \|110> |
| Patience | D | 0 | 1 | 0 | 010 | \|010> |
| Patience | E | 0 | 0 | 1 | 001 | \|001> |
| Patience | F | 1 | 0 | 1 | 101 | \|101> |
| Patience | G | 0 | 1 | 1 | 011 | \|011> |
| Patience | H | 1 | 1 | 1 | 111 | \|111> |
| Patience | I | 1 | 0 | −1 | 10-1 | −\|101> |
| Patience | J | 0 | 0 | −1 | 00-1 | −\|001> |
| Patience | K | 1 | 1 | −1 | 11-1 | −\|111> |
| Patience | L | 0 | 1 | −1 | 01-1 | −\|011> |

| column8 | column9 |
|---|---|
| charge field coordinate (x-axis) | fundemental category discription choice |
| more | B |
| less | B |
| more | B |
| less | B |
| more | B |
| less | B |
| more | B |
| less | B |
| more | B |
| less | B |
| more | B |
| less | B |
| more | A |
| less | A |
| more | A |
| less | A |
| more | A |
| less | A |
| more | A |
| less | A |
| more | A |
| less | A |
| more | A |
| less | A |
| more | B |
| less | B |
| more | B |
| less | B |
| more | B |
| less | B |
| more | B |
| less | B |
| more | B |
| less | B |
| more | B |
| less | B |
| more | B |
| less | B |
| more | B |
| less | B |
| more | B |
| less | B |
| more | B |
| less | B |
| more | B |
| less | B |
| more | B |
| less | B |

column10
combined inqury
What do you observe in your strength of being able to handle ambiguous situations well?
What do you appreciate in others strength of being sees issues in terms of black or white?
How do you admire your strength of being able to handle ambiguous situations well?
How do you respect others strength of being sees issues in terms of black or white?
What will you appreciate in your strength of being able to handle ambiguous situations well?
What will you observe in others strength of being sees issues in terms of black or white?
How will you respect your strength of being able to handle ambiguous situations well?
How will you admire others strength of being sees issues in terms of black or white?
What did you observe in your strength of being able to handle ambiguous situations well?
What did you appreciate in others strength of being sees issues in terms of black or white?
How did you admire your strength of being able to handle ambiguous situations well?
How did you respect others strength of being sees issues in terms of black or white?
What do you acknowledge in your interest in using imagination and intuitiveness?
What do you consider in others interest in approaching problems factually and logically?
How do you fulfill your interest in using imagination and intuitiveness?
How do you discover others interest in approaching problems factually and logically?
What will you consider in your interest in using imagination and intuitiveness?
What will you acknowledge in others interest in approaching problems factually and logically?
How will you discover your interest in using imagination and intuitiveness?
How will you fulfill others interest in approaching problems factually and logically?
What did you acknowledge in your interest in using imagination and intuitiveness?
What did you consider in others interest in approaching problems factually and logically?
How did you fulfill your interest in using imagination and intuitiveness?
How did you discover others interest in approaching problems factually and logically?
What do you support in your need for plenty of time to make complex decisions?
What do you allow in others need for reducing issues to their simplest form?
How do you maintain your need for plenty of time to make complex decisions?
How do you accept others need for reducing issues to their simplest form?
What will you allow in your need for plenty of time to make complex decisions?
What will you support in others need for reducing issues to their simplest form?
How will you accept your need for plenty of time to make complex decisions?
How will you maintain others need for reducing issues to their simplest form?
What did you support in your need for plenty of time to make complex decisions?
What did you allow in others need for reducing issues to their simplest form?
How did you maintain your need for plenty of time to make complex decisions?
How did you accept others need for reducing issues to their simplest form?
*Hint: comprehend your stress toward having indecision when pressured?
*Hint: excuse others stress toward being impulsive?
*Hint: understand your stress toward having indecision when pressured?
*Hint: forgive others stress toward being impulsive?
*Hint: excuse your stress toward having indecision when pressured?
*Hint: comprehend others stress toward being impulsive?
*Hint: forgive your stress toward having indecision when pressured?
*Hint: understand others stress toward being impulsive?
*Hint: what did you comprehend in your stress toward having indecision when pressured?
*Hint: what did you excuse in others stress toward being impulsive?
*Hint: how did you understand your stress toward having indecision when pressured?
*Hint: how did you forgive others stress toward being impulsive?

| column11 | column12 | column13 | column14 |
|---|---|---|---|
| energy level inquiry | motivational coping technique | prefix inquiry | motivational attribute |
| component | inquiry component | component | inquiry component |
| What do you | observe | in | your strength of being |
| What do you | appreciate | in | others strength of being |
| How do you | admire | | your strength of being |
| How do you | respect | | others strength of being |
| What will you | appreciate | in | your strength of being |
| What will you | observe | in | others strength of being |
| How will you | respect | | your strength of being |
| How will you | admire | | others strength of being |
| What did you | observe | in | your strength of being |
| What did you | appreciate | in | others strength of being |
| How did you | admire | | your strength of being |
| How did you | respect | | others strength of being |
| What do you | acknowledge | in | your interest in |
| What do you | consider | in | others interest in |
| How do you | fulfill | | your interest in |
| How do you | discover | | others interest in |
| What will you | consider | in | your interest in |
| What will you | acknowledge | in | others interest in |
| How will you | discover | | your interest in |
| How will you | fulfill | | others interest in |
| What did you | acknowledge | in | your interest in |
| What did you | consider | in | others interest in |
| How did you | fufill | | your interest in |
| How did you | discover | | others interest in |
| What do you | support | in | your need for |
| What do you | allow | in | others need for |
| How do you | maintain | | your need for |
| How do you | accept | | others need for |
| What will you | allow | in | your need for |
| What will you | support | in | others need for |
| How will you | accept | | your need for |
| How will you | maintain | | others need for |
| What did you | support | in | your need for |
| What did you | allow | in | others need for |
| How did you | maintain | | your need for |
| How did you | accept | | others need for |
| *Hint: | comprehend | | your stress toward |
| *Hint: | excuse | | others stress toward |
| *Hint: | understand | | your stress toward |
| *Hint: | forgive | | others stress toward |
| *Hint: | excuse | | your stress toward |
| *Hint: | comprehend | | others stress toward |
| *Hint: | forgive | | your stress toward |
| *Hint: | understand | | others stress toward |
| *Hint: what did you | comprehend | in | your stress toward |
| *Hint: what did you | excuse | in | others stress toward |
| *Hint: how did you | understand | | your stress toward |
| *Hint: how did you | forgive | | others stress toward |

| | |
|---|---|
| column15 | column16 |

discription inquiry component

| more like description inquiry component | less like description inquiry component |
|---|---|
| able to handle ambiguous situations well | sees issues in terms of black or white |
| able to handle ambiguous situations well | sees issues in terms of black or white |
| able to handle ambiguous situations well | sees issues in terms of black or white |
| able to handle ambiguous situations well | sees issues in terms of black or white |
| able to handle ambiguous situations well | sees issues in terms of black or white |
| able to handle ambiguous situations well | sees issues in terms of black or white |
| able to handle ambiguous situations well | sees issues in terms of black or white |
| able to handle ambiguous situations well | sees issues in terms of black or white |
| able to handle ambiguous situations well | sees issues in terms of black or white |
| able to handle ambiguous situations well | sees issues in terms of black or white |
| able to handle ambiguous situations well | sees issues in terms of black or white |
| able to handle ambiguous situations well | sees issues in terms of black or white |
| using imagination and intuitiveness | approaching problems factually and logically |
| using imagination and intuitiveness | approaching problems factually and logically |
| using imagination and intuitiveness | approaching problems factually and logically |
| using imagination and intuitiveness | approaching problems factually and logically |
| using imagination and intuitiveness | approaching problems factually and logically |
| using imagination and intuitiveness | approaching problems factually and logically |
| using imagination and intuitiveness | approaching problems factually and logically |
| using imagination and intuitiveness | approaching problems factually and logically |
| using imagination and intuitiveness | approaching problems factually and logically |
| using imagination and intuitiveness | approaching problems factually and logically |
| using imagination and intuitiveness | approaching problems factually and logically |
| using imagination and intuitiveness | approaching problems factually and logically |
| plenty of time to make complex decisions | reducing issues to their simplest form |
| plenty of time to make complex decisions | reducing issues to their simplest form |
| plenty of time to make complex decisions | reducing issues to their simplest form |
| plenty of time to make complex decisions | reducing issues to their simplest form |
| plenty of time to make complex decisions | reducing issues to their simplest form |
| plenty of time to make complex decisions | reducing issues to their simplest form |
| plenty of time to make complex decisions | reducing issues to their simplest form |
| plenty of time to make complex decisions | reducing issues to their simplest form |
| plenty of time to make complex decisions | reducing issues to their simplest form |
| plenty of time to make complex decisions | reducing issues to their simplest form |
| plenty of time to make complex decisions | reducing issues to their simplest form |
| plenty of time to make complex decisions | reducing issues to their simplest form |
| having indecision when pressured | being impulsive |
| having indecision when pressured | being impulsive |
| having indecision when pressured | being impulsive |
| having indecision when pressured | being impulsive |
| having indecision when pressured | being impulsive |
| having indecision when pressured | being impulsive |
| having indecision when pressured | being impulsive |
| having indecision when pressured | being impulsive |
| having indecision when pressured | being impulsive |
| having indecision when pressured | being impulsive |
| having indecision when pressured | being impulsive |
| having indecision when pressured | being impulsive |

What is claimed is:

1. A method for facilitating personal development using a computing device by providing non-transitory machine-readable instructions on a tangible medium to perform steps, comprising:
for each user of a plurality of users, perform the following collections of steps (1) through (13) by employing a computational system operatively configured for entering into a dialog with each of a plurality of users:
(1) obtaining and storing, via a computer of a computational system operatively configured for entering into a corresponding dialogue with each of the plurality of users, data for each of one or more predetermined user motivations, each motivation identified as a driver of user perceptions for the plurality of users, wherein the data includes a set of one or more words descriptive of each of the user motivations; wherein step (1) includes a step of receiving the data for each of the one or more predetermined user motivations, via a computer display of a computational system operatively configured for entering into a corresponding dialogue with each of a plurality of users;
(2) for each corresponding portion of the data for the user motivations, additional steps of obtaining and storing, through a computer display of the computational system operatively configured for entering into a corresponding dialogue with each of a plurality of users, a corresponding portion of personal description data for the user, the corresponding portion of the personal description data having a plurality of user specific descriptions, each of the user specific descriptions being both: (A) specific to the user, and descriptive of personal traits describing how to relate to a situation of concern, and (B) related to the corresponding portion of the data for the user motivations, and sufficiently meaningful to the user to assist the user in his/her personal growth and development when the user specific description is presented to the user as an example of the corresponding portion of the data for the user motivations;
grouping, for each corresponding portion of the data for the user motivations, the user specific descriptions in the users corresponding portion of personal description data by a second computational component of the computational system operatively configured for entering into a corresponding dialogue with each of a plurality of users, wherein each of the user specific descriptions is grouped according to: (i) whether the user specific description is more characteristic of the user or less characteristic of the user, and (ii) whether the user is more aware of the user specific description being characteristic of the user or less aware of the user specific description being characteristic of the user, the resulting groups being as (2-a) through (2-c) following:
(2-a) a first group for one or more of the user specific descriptions, each such user specific description in the first group describing a corresponding personal attribute that is: (i) more characteristic of the user, and (ii) the user is more aware of the corresponding attribute being characteristic of the user,
(2-b) a second group for one or more of the user specific descriptions, each such user specific description in the second group describing a corresponding personal attribute that is: (i) less characteristic of the user, and (ii) the user is more aware of the corresponding attribute, recited immediately above, being less characteristic of the user, and
(2-c) a third group for one or more of the user specific descriptions, each such user specific description in the third group describing a corresponding personal attribute that is: (i) more characteristic of the user, and (ii) the user is less aware of the corresponding attribute, recited immediately above, being more characteristic of the user;
(3) obtaining and storing for each of the user motivations, data indicative of each of a plurality of coping techniques, each coping technique describing a technique for use by the user in relating to another person or situation, wherein for each coping technique, the data therefor identifies a corresponding second set of one or more words describing the coping technique, and for at least some of the coping techniques, their corresponding second set of words are different;
wherein a substep is performed of classifying the data indicative of the coping techniques into the following data classifications (3-a) through (3-c):
(3-a) a first data classification that includes the data for a first one or more of the coping techniques, wherein for each of the first one or more coping techniques, the data therefor is: (i) associated, in a step of associating, with each of the first and second groups, and (ii) associated with additional data that identifies the coping technique as initiating more activities to change how the user relates to issues involving another person or situation rather than meditatively reflecting on the issues;
(3-b) a second data classification that includes data for a second one or more of the coping techniques, wherein for each of the second one or more coping techniques, the data therefor is: (i) associated, in a step of associating, with each of the third and fourth groups, and (ii) associated with additional data that identifies the coping technique as initiating more activities to change how the user relates to the issues involving another person or situation rather than meditatively reflecting on the issues;
(3-c) a third data classification that includes data for a third one or more of the coping techniques, wherein for each of the third one or more coping techniques, the data therefor is: (i) associated, in a step of associating, with each of the first and second groups, and (ii) associated with additional data that identifies the coping technique as monitoring and evaluating how the user relates to the issues involving another person or situation rather than initiating activities to change how the user relates to the issues;
(4) obtaining, via a computational system operatively configured for entering into a corresponding dialogue with each of a plurality of users, input for a topic identifying a target of concern to the user;
performing for each one of the user motivations the following collections of step (5) through (13):
(5) outputting to the user a request for the user to input an evaluation as to how the user relates to the target of concern relative to the one user motivation; wherein the step of outputting includes a step of presenting topic related data to the user via a computer display of the a computational system operatively configured for entering into a corresponding dialogue with each of a plurality of users to assist the user in his or her personal growth and development;
(6) receiving, via a computational system operatively configured for entering into a corresponding dialogue with each of a plurality of users, the evaluation indicative of the user's perception as to how the user relates to the target of concern, and a degree of confidence the user has in the evaluation;
(7) ranking, by a computational system operatively configured for entering into a corresponding dialogue with each of a plurality of users, the first, second, and third groups of user specific descriptions, for the one user motivation, using the evaluation and the degree of confidence, wherein the ranking assigns a ranking value for each of the first, second, and third groups, wherein for each of the groups the ranking value therefor is indicative of how well the user specific descriptions of the group in each of the first, second, and third groups relate to the user's perspective of the target of concern for assisting the user in his/her personal growth and development;
wherein the step of ranking is performed in response to an input to a computer display of the computational system;
(8) determining a primary one of the coping techniques for the one user motivation by the substeps (8-a) and (8-b):
(8-a) obtaining a plurality of assignments, for each of the first, second, and third data classifications for the one user motivation, one of the assignments being a pairing of: (i) the data for one of the coping techniques for the data classification, with (ii) a selected one of the associated groups for the one coping technique, wherein the selection is dependent upon ranking values; and
(8-b) selecting the primary coping technique as the coping technique wherein the data therefor is paired with a particular one of the first, second, and third groups, wherein the particular group is determined according to a result of a predetermined function of the ranking values;
(9) generating, based on the ranking, via a computational system operatively configured for entering into a corresponding dialogue with each of a plurality of users, a responsive presentation to the user in a non-transitory form that provides the user with topic related information to assist the user in his or her personal growth and development, the generating step including providing a presentation generator for generating presentations for interacting with a user and providing textual descriptions organized so that outputs to the user are generated from various personality/motivational perspectives, the responsive presentation including:
(9a) the set of one or more words descriptive of the one user motivation;
(10) electronically outputting the responsive presentation to the user as a computer display;
(11) electronically receiving a responsive user input inputted into a computer display of a computational system operatively configured for entering into a corresponding dialogue with each of a plurality of users, wherein the responsive user input is a response to the responsive presentation to the user;
(12) displaying the input for a topic identifying a target of concern to the user;
(13) storing, in non-transitory computer storage, linked data including: the responsive presentation, and the responsive user input, wherein the responsive presentation and the responsive user input are linked together for electronic access by a computational system operatively configured for entering into a corresponding dialogue with each of a plurality of users;
wherein the stored linked data is available for outputting the responsive presentation, and the responsive user input for assisting the user with his/her personal growth and development; and linking each user to an audio file, said audio file comprising a visualization exercise;
wherein said one or more predetermined user motivations include an attitudinal trait description;
wherein said attitudinal trait description represents how a user most likely behaves or least likely behaves and wherein the attitudinal trait description is consistent with a user profile.
